(19) United States
(12) Patent Application Publication
(10) Pub. No.: US 2025/0135034 A1
(43) Pub. Date: May 1, 2025

(54) CRISPR-MEDIATED MANIPULATION OF APP EXPRESSION AS A THERAPEUTIC APPROACH FOR AMYLOID PATHOLOGIES

(71) Applicants: The Hong Kong University of Science & Technology, Kowloon, Hong Kong (CN); HONG KONG CENTER FOR NEURODEGENERATIVE DISEASES LIMITED, Hong Kong (CN)

(72) Inventors: Nancy Yuk-Yu Ip, Hong Kong (CN); Kit Yu Fu, Kowloon, Hong Kong SAR (CN); Yangyang Duan, Kunming (CN); Tao Ye, Shenzhen (CN)

(21) Appl. No.: 18/691,303

(22) PCT Filed: Oct. 28, 2022
(86) PCT No.: PCT/CN2022/128123

§ 371 Date: Mar. 12, 2024

Related U.S. Application Data
(60) Provisional application No. 63/272,689, filed on Oct. 28, 2021.
(60) Provisional application No. 63/334,650, filed on Apr. 25, 2022.

(30) Foreign Application Priority Data
Oct. 28, 2022 (WO) PCTCN2022128123

(51) Int. Cl. A61K 48/00; C12N 9/22; C12N 15/11; C12N 15/86; C12Q 1/6883

(52) U.S. Cl. CPC A61K 48/0058; C12N 9/22; C12N 15/11; C12N 15/86; C12Q 1/6883; C12N 2750/14143; C12Q 2600/156

(57) ABSTRACT

Compositions and methods are provided for the treatment or prophylaxis of amyloid pathologies such as Alzheimer's Disease.

### RELATED APPLICATIONS

This application claims priority to U.S. Provisional Patent Application No. 63/272,689, filed Oct. 28, 2021, and U.S. Provisional Patent Application No. 63/334,650, filed Apr. 25, 2022, the contents of both of which are hereby incorporated by reference in the entirety for all purposes.

### BACKGROUND OF THE INVENTION

Brain diseases such as neurodegenerative diseases and neuroinflammatory disorders are devastating conditions that affect a large subset of the population. Many of such diseases are incurable, highly debilitating, and often result in progressive deterioration of the brain structure and function over time. Disease prevalence is also increasing rapidly due to growing aging populations worldwide, since the elderly are at high risk for developing these conditions. Currently, many neurodegenerative diseases and neuroinflammatory disorders are difficult to diagnose due to limited understanding of the pathophysiology of these diseases. Meanwhile, current treatments are ineffective and do not meet market demand; demand that is significantly increasing each year due to the ever-growing aging populations. For example, Alzheimer's disease (AD) is marked by gradual but progressive decline in learning and memory, and a leading cause of mortality in the elderly. Increasing prevalence of AD is driving the need and demand for better and earlier diagnostics. According to Alzheimer's Disease International, the disease currently affects 46.8 million people globally, but the number of cases is projected to triple in the coming three decades. One of the countries with the fastest elderly population growth is China. Based on population projections, by 2030 one in four individuals will be over the age of 60, which will place a vast proportion at risk of developing AD. In fact, the number of AD cases in China doubled from 3.7 million to 9.2 million from 1990-2010, and the country is projected to have 22.5 million cases by 2050. Hong Kong's population is also aging quickly. It is estimated that the elderly aged 65+ will make up 24% of the population by 2025, and 39.3% of the population by 2050. The number of AD cases is projected to rise to 332,688 by 2039. As such, there exists an urgent need for developing new methods for effectively treating AD patients who suffer from this devastating condition. This invention fulfills this and other related needs by disclosing novel compositions and methods useful for effective treatment, and potentially providing a cure, of the disease.

### BRIEF SUMMARY OF THE INVENTION

In a first aspect, this invention provides a method for treating an amyloid pathology (such as Alzheimer's Disease) in a person or reducing the person's risk for later developing the pathology. The claimed method comprises the step of administering to the person an effective amount of a composition disrupting a genomic sequence encompassing the APP gene coding sequence or transcript.

In some embodiments, the claimed method comprises, prior to the administering step, sequencing at least a portion of the person's genome. In some embodiments, the person's APP genomic sequence comprises at least one mutation, for example, a Swedish mutation (K670N/M671L), a Florida mutation (I716V), a London mutation (V717I), or any combination thereof, as well as other genes harboring mutations or allelic variants relevant to amyloid plaque formation. In some embodiments, the mutation comprises a Swedish mutation (K670N/M671L). In some embodiments, the amyloid pathology is Alzheimer's Disease (AD, including both familial and sporadic AD), cerebral amyloid angiopathy, or Down Syndrome. In some embodiments, the person has been diagnosed with AD, whereas in other embodiments, the person is not yet diagnosed with AD but has known risk factors for AD, such as harboring certain genetic mutations known to increase one's risk for AD or having a family history of AD, especially early onset AD (e.g., before 60 or 70 years of age). In some embodiments, the method of this invention is practiced using a composition comprising an siRNA, a microRNA, a miniRNA, a lncRNA, or an antisense oligonucleotide targeting the APP genomic sequence. In some embodiments, the method utilizes a CRISPR composition comprising one or more vectors encoding (1) an endonuclease guided by a small guide RNA (sgRNA) targeting a location within the APP genomic sequence; and (2) the sgRNA. In some embodiments, the sgRNA targets a region within the APP genomic sequence comprising a known mutation (e.g., the Swedish mutation) or a β- or γ-secretase cleavage site, for example, the targeted endonuclease cleavage site is within 2, 3, 5, 7, 8, 9, 10, 11, 12, 15 nucleotides upstream or downstream from the mutation or secretase cleavage site. In some embodiments, the composition comprises one vector encoding a Cas9 nuclease and one sgRNA, e.g., a Streptococcus pyogenes Cas9 nuclease (SpCas9). In some embodiments, the Cas9 and sgRNA are operably linked to and under the transcription control of a tissue-specific promoter, such as a neuronal cell-specific promoter known in the technical field or disclosed herein. In some embodiments, each of the one or more vectors is one viral vector, preferably an adeno-associated virus (AAV) vector. In some embodiments, the composition is administered by subcutaneous, intramuscular, intravenous, intraperitoneal, or intracranial injection or by oral or nasal administration. In some embodiments, the composition is administered in the form of a solution, a suspension, a powder, a paste, a tablet, or a capsule.

In a second aspect, the present invention provides a composition comprising an effective amount of one or more agents that disrupt the APPP genomic sequence plus one or more physiologically acceptable excipient.

In some embodiments, the composition comprising an siRNA, a microRNA, a miniRNA, a lncRNA, or an antisense oligonucleotide targeting the APP genomic sequence. In some embodiments, the method utilizes a CRISPR composition comprising one or more vectors encoding (1) an endonuclease guided by a small guide RNA (sgRNA) targeting a location within the APP genomic sequence; and (2) the sgRNA. In some embodiments, the sgRNA targets a region within the APP genomic sequence comprising a known mutation (e.g., the Swedish mutation) or a β- or γ-secretase cleavage site, for example, the targeted endonuclease cleavage site is within 2, 3, 5, 7, 8, 9, 10, 11, 12, 15 nucleotides upstream or downstream from the mutation or secretase cleavage site. In some embodiments, the composition comprises one vector encoding a Cas9 nuclease and one sgRNA, e.g., a Streptococcus pyogenes Cas9 nuclease (SpCas9). In some embodiments, the Cas9 and sgRNA are operably linked to and under the transcription control of a tissue-specific promoter, such as a neuronal cell-specific promoter known in the technical field or disclosed herein. In some embodiments, each of the one or more vectors is one viral vector, preferably an adeno-associated virus (AAV) vector. In some embodiments, the composition is administered by subcutaneous, intramuscular, intravenous, intraperitoneal, or intracranial injection or by oral or nasal administration. In some embodiments, the composition is administered in the form of a solution, a suspension, a powder, a paste, a tablet, or a capsule.

In a third aspect, the present invention provides a kit for treating an amyloid pathology (such as Alzheimer's Disease) in a person or for reducing the person's risk of later developing the disease/condition. The kit comprises a first container containing a composition disrupting APP genomic sequence.

In some embodiments, the composition is formulated for subcutaneous, intramuscular, intravenous, intraperitoneal, or intracranial injection, or for oral or nasal administration. In some embodiments, the composition comprising an siRNA, a microRNA, a miniRNA, a lncRNA, or an antisense oligonucleotide targeting the APP genomic sequence. In some embodiments, the composition comprises (1) one or more vectors encoding an endonuclease guided by a small guide RNA (sgRNA) and (2) one sgRNA (or two sgRNAs) targeting one (or two locations) within the APP genomic sequence, for example, in a small region in close proximity to a pre-selected mutation (e.g., the Swedish mutation) or a β- or γ-secretase cleavage site within the APP genomic sequence, so that the intended endonuclease cleavage site is within 2, 3, 5, 7, 8, 9, 10, 11, 12, 15 nucleotides upstream or downstream from the mutation or secretase cleavage site. In some embodiments, the kit contains a composition comprising one single vector encoding a Cas9 nuclease, for example, a Streptococcus pyogenes Cas9 nuclease (SpCas9), and one or more sgRNAs. In some embodiments, the endonuclease and the sgRNA(s) may be encoded in two separate vectors. In some embodiments, the vector is a viral vector, such as one constructed based on an adenovirus or an adeno-associated virus (AAV). In some embodiments, the composition is administered by subcutaneous, intramuscular, intravenous, intraperitoneal, or intracranial injection or by oral or nasal administration. In some embodiments, the kit further contains a second container containing agents for sequencing at least a portion of the person's genome (e.g., the APP gene as well as other genes that might harbor mutations or variant known to play a role in amyloid pathogenesis such as presenilin 1 (PS1) and presenilin 2 (PS2), and APOE-&4). Optionally, the kit also includes an instruction manual for administration of the composition.

Related to this aspect of the present invention, a use of one or more agents disrupting APP genomic sequence is further provided in accordance with the disclosure herein for the manufacturing of (1) a medicament for treating an amyloid pathology (e.g., Alzheimer's Disease); and/or (2) a kit containing the medicament for treating the amyloid pathology (e.g., Alzheimer's Disease).

### BRIEF DESCRIPTION OF THE DRAWINGS

FIG. 1: Design and validation of CRISPR/Cas9-mediated genome editing to disrupt the mutant APP_{swe} allele. a, The APP Swedish (APP_{swe}) double-base mutation (KM670/671NL) is close to the β-secretase site. Two single-guide RNAs (sgRNAs)—SW1 and SW2—were designed to target the APP_{swe} mutation using Staphylococcus aureus Cas9 (SaCas9). PAM, protospacer-adjacent motif. The arrowhead indicates the SaCas9 cleavage site. b, Schematic diagram of the EGxxFP system used to analyze the editing efficiency of the CRISPR/Cas9 system in vitro. The APP_{WT} and APP_{swe} sequences were separately inserted between EGFP fragments with overlapping arms. SaCas9 (blue) editing resulted in homology-directed repair (HDR)-mediated DNA repair and reconstitution of the EGFP expression cassette, which yielded GFP signals. c, In vitro validation of allele-specific editing using the EGxxFP system. Cas9-SW1 and Cas9-SW2 were co-transfected with EGxxFP reporter plasmids containing APP_{WT} or APP_{swe} (EGxxFP-APP_{WT} or EGxxFP-APP_{swe}), respectively, into HEK 293T cells. Scale bar: 100 μm. d, A single adeno-associated virus (AAV) vector, EFS::Cas9-SW1, was constructed to express HA-tagged SaCas9 and sgRNA SW1 for intrahippocampal injection. EFS, elongation factor 1-alpha short promoter; pA, poly(A) tail; U6, human U6 promoter; ITR, inverted terminal repeat. e, T7 endonuclease I assay for AAV-mediated Cas9-SW1 editing in virus-transduced hippocampi from 5XFAD mice. Arrowheads indicate the CRISPR/Cas9-edited DNA fragments. Con, Untransduced hippocampal region of 5XFAD mice. f, Percentage of insertion/deletion (indel) mutations. CRISPR/Cas9 editing led to the formation of indel mutations. The indel percentage indicates the editing efficiency. Values are mean±SEM (n=3 mice per group; *** P<0.001; unpaired two-tailed t-test).

FIG. 2: Intrahippocampal AAV-mediated Cas9-SW1 editing decreases amyloid plaque burden in 5XFAD mice. Intrahippocampal injection of AAV-mediated Cas9-SW1 in 3-month-old 5XFAD mice decreased amyloid-beta (Aβ) levels and deposition. a-c, Soluble and insoluble Aβ contents in the dorsal hippocampal homogenates of 6-month-old 5XFAD mice after intrahippocampal injection of Cas9-SW1. a, Representative western blots of soluble and insoluble Aβ with GAPDH as a loading control (n=3 mice per group). b, c, Quantitative analysis of soluble and insoluble Aβₓ₋₄₀ and Aβₓ₋₄₂ by ELISA. Values are mean±SEM (n=5 mice per group; * P<0.05, ** P<0.01, *** P<0.001; unpaired two-tailed t-test). d-k, Amyloid plaque deposition decreased in the hippocampal regions in 5XFAD mice that expressed AAV-mediated Cas9-SW1. Immunohistochemistry for Aβ (4G8; green), HA (red), and nuclei (blue). Representative images of distinct hippocampal regions (i.e., the cornu ammonis and subiculum) of 5XFAD mice at 6 months (d, e) and 9 months (h, i). HA tag expression indicates Cas9 expression. Scale bars: 50 μm. Quantification of Aβ-positive areas in the cornu ammonis (f, j) and subiculum (g, k). Values are mean±SEM (n=4 mice per group; * P<0.05, ** P<0.01; paired two-tailed t-test).

FIG. 3: Intrahippocampal AAV-mediated Cas9-SW1 editing decreases gliosis in 5XFAD mice. a, b, AAV-mediated Cas9-SW1 editing decreased microgliosis in the hippocampus of 5XFAD mice. Immunohistochemistry for Iba1 (red), amyloid-beta (Aβ, 4G8; green), and nuclei (blue) in 6-month-old 5XFAD mice. a, Representative images of the subiculum. Scale bars: 50 μm (left), 10 μm (right). b, Quantification of Iba1-positive cells. Values are mean±SEM (n=3 mice per group; * P<0.05; paired two-tailed t-test). c-e, AAV-mediated Cas9-SW1 editing decreased the activation of astrocytes in the hippocampus in 5XFAD mice. Immunohistochemistry for GFAP (red), Aβ (4G8; green), and nuclei (blue) of 6-month-old 5XFAD mice. c, Representative images of the subiculum. Scale bars: 50 μm (left), 10 μm (right). d, e, AAV-mediated Cas9-SW1 editing in 5XFAD mice did not change the number of astrocytes but decreased astrocyte activation. d, Quantification of GFAP-positive cells. Values are mean±SEM (n=4 mice per group; paired two-tailed t-test). e, Percentages of GFAP-positive areas. Values are mean±SEM (n=4 mice per group; * P<0.05; paired two-tailed t-test).

FIG. 4: Intrahippocampal AAV-mediated Cas9-SW1 editing improves neuronal functions in 5XFAD mice. a, b, AAV-mediated Cas9-SW1 editing reversed the decrease of long-term potentiation (LTP) in 6-month-old 5XFAD mice. LTP in the hippocampal CA1 region was induced by 4 trains of theta-burst stimulation. WT, wild type. a, Averaged slopes of baseline normalized field excitatory postsynaptic potential (fEPSP; mean±SEM). (Inset) Examples of fEPSPs recorded 5 min before (1, gray) and 55 min after (2, black) LTP induction. b, Quantification of mean fEPSP slopes during the last 10 min of recording after LTP induction. Values are mean±SEM (WT Con n=9 slices from 6 mice; 5XFAD Con n=9 slices from 4 mice; 5XFAD SW1 n=9 slices from 5 mice; ** P<0.01, *** P<0.001; unpaired two-tailed t-test). c-e, AAV-mediated Cas9-SW1 injection reduced the decrease of excitatory synapses (stained with PSD-95 [green] and synaptophysin [red]) in the subiculum in 9-month-old 5XFAD mice. c, Representative images. Scale bar: 5 μm. d, e, Quantification of PSD-95 puncta, synaptophysin puncta, and synaptophysin-PSD-95 co-localized puncta in the subiculum. Values are mean±SEM (n=4 mice per group; * P<0.05; paired two-tailed t-test). f-i, AAV-mediated Cas9-SW1 editing decreased neurite dystrophy and neuronal loss in the subiculum in 5XFAD mice. Co-staining with amyloid-beta (Aβ, 4G8; green), nuclei (blue), and LAMP1 (a marker of neurite dystrophy; red) in 6-month-old 5XFAD mice (f) or NeuN (red) in 9-month-old 5XFAD mice (g). Scale bars: 50 μm (f), 200 μm (g). h, Quantification of LAMP1-positive dystrophic neurites. Values are mean±SEM (n=3 mice per group; paired two-tailed t-test). i, Quantification of NeuN-positive cell density. Values are mean±SEM (n=4 mice per group; * P<0.05; paired two-tailed t-test).

FIG. 5: Intrahippocampal AAV-mediated delivery of Cas9-SW1 decreases Alzheimer's disease-associated pathologies in APP/PS1 mice. Intrahippocampal injection of AAV-mediated Cas9-SW1 in 9-month-old APP/PS1 mice decreased amyloid-beta (Aβ) deposition and gliosis. a-d, Amyloid plaque deposition decreased in the hippocampal regions in APP/PS1 mice that expressed AAV-mediated Cas9-SW1. a, b, Immunohistochemistry for Aβ (4G8; green), HA (red), and nuclei (blue) in 15-month-old APP/PS1 mice. Scale bar: 50 μm. Quantification of Aβ-positive plaque areas in the cornu ammonis (c) and subiculum (d). Values are mean±SEM (n=4 mice per group; * P<0.05, ** P<0.01; paired two-tailed t-test). e-h, AAV-mediated Cas9-SW1 editing decreased gliosis in the subiculum in APP/PS1 mice. e, Immunohistochemistry for microglial marker Iba1 (red), Aβ (4G8; green), and nuclei (blue). Scale bars: 50 μm (left), 10 μm (right). f, Quantification of Iba1-positive cells. Values are mean±SEM (n=4 mice per group; * P<0.05; paired two-tailed t-test). g, Immunohistochemistry for the astrocyte marker GFAP (red), Aβ (4G8; green), and nuclei (blue) in APP/PS1 mice. Scale bars: 50 μm (left), 10 μm (right). h, Quantification of GFAP-positive cells. Values are mean±SEM (n=4 mice per group; * P<0.05; paired two-tailed t-test). i, Percentages of GFAP-positive areas. Values are mean±SEM (n=4 mice per group; *P<0.05; paired two-tailed t-test).

FIG. 6: Systemic delivery of AAV-PHP.eB-mediated Cas9-SW1 globally decreases amyloid plaque burden in 5XFAD mice. a, Diagram of brain-wide APP_{swe} editing. A single AAV vector, designated SW1-Syn::Cas9-mWPRE, was designed to express HA-tagged SaCas9 and sgRNA SW1 for systemic administration using an AAV-PHP.eB capsid. ITR, inverted terminal repeat; U6, human U6 promoter; Syn, human synapsin 1 promoter; mWPRE, truncated form of Woodchuck Hepatitis Virus (WHP) Posttranscriptional Regulatory Element (WPRE); pA, poly(A) tail. b-d, Systemic injection of AAV-PHP.eB-mediated Cas9-SW1 into 3-month-old 5XFAD mice globally decreased the amyloid-beta (Aβ)-positive plaque area in the brain. b, Representative images of amyloid plaque (i.e., 4G8) staining in 6-month-old 5XFAD mice after AAV-PHP.eB: SW1-Syn::Cas9-mWPRE injection. Quantification of amyloid plaque areas in the cortex (c) and hippocampus (d). Values are mean±SEM (n=4 mice per group; * P<0.05, ** P<0.01; unpaired two-tailed t-test). e-l, The decrease in X34-labeled amyloid plaque burden was associated with Cas9 expression in the brain. e, f, i, j, Immunohistochemistry for X34 (green), HA (red), and nuclei (blue) in the cornu ammonis (e), subiculum (f), cortex (i), and medulla (j) in 6-month-old 5XFAD mice. HA tag expression indicates Cas9 expression. Scale bar: 50 μm. g, h, k, l, Quantification of X34-labeled plaque areas. Values are mean±SEM (n=4 mice per group; * P<0.05, ** P<0.01; unpaired two-tailed t-test).

FIG. 7: Systemic delivery of AAV-PHP.eB-mediated Cas9-SW1 decreases microgliosis and neurite dystrophy, and improves cognitive performance in 5XFAD mice. a, b, Systemic delivery of AAV-PHP.eB-mediated Cas9-SW1 in 3-month-old 5XFAD mice decreased microgliosis in the subiculum. a, Immunohistochemistry for Iba1 (red), amyloid-beta (Aβ, 4G8; green), and nuclei (blue) in 6-month-old 5XFAD mice. Scale bars: 50 μm (left), 10 μm (right). b, Quantification of Iba1-positive microglia. Values are mean±SEM (n=4 mice per group; * P<0.05; unpaired two-tailed t-test). c, d, AAV-PHP.eB-mediated Cas9-SW1 editing decreased neurite dystrophy in 5XFAD mice. c, Representative images showing staining for LAMP1 (a marker of neurite dystrophy; red), Aβ (4G8; green), and nuclei (blue) in the subiculum in 6-month-old 5XFAD mice. Scale bar: 10 μm. d, Quantification of LAMP1-positive dystrophic neurites. Values are mean±SEM (n=4 mice per group; ** P<0.01; unpaired two-tailed t-test). e-g, Systemic delivery of AAV-PHP.eB-mediated Cas9-SW1 improved the cognitive performance of 5XFAD mice. e, Systemic delivery of AAV-PHP.eB-mediated Cas9-SW1 improved habituation in the exploratory open field (OF) test. Percentage change in the distance traveled relative to that traveled on the first day of training. Values are mean±SEM (n=12 mice per group; * P<0.05, ** P<0.01; two-way repeated-measures ANOVA). f, Systemic delivery of AAV-PHP.eB-mediated Cas9-SW1 improved spatial working memory in the spontaneous alteration Y-maze test. Values are mean±SEM (n=12 mice per group; * P<0.05, ** P<0.01; unpaired two-tailed t-test). g, Upon systemic delivery of AAV-PHP.eB-Cas9-SW1, the 5XFAD mice spent significantly less time in the open arms. Values are mean±SEM (n=12 mice per group; * P<0.05; unpaired two-tailed t-test).

FIG. 8: Validation of allele-specific editing in HEK 293T cells and 5XFAD mice. a, b, Cas9-SW1 and Cas9-SW2 did not target APP_{WT}. a, T7 endonuclease I mismatch assay for CRISPR/Cas9-mediated genome editing in HEK 293T cells carrying APP_{WT} alleles. WT1 and WT2 are single-guide RNAs (sgRNAs) that target the APP_{WT} allele at the same genetic loci as those of SW1 and SW2, respectively. b, Insertion/deletion (indel) rates were examined by targeted deep sequencing. Values are mean±SEM (n=5 replicates per group; *** P<0.001; unpaired Student's t-test). c, d, Off-target analysis of Cas9-SW1. c, Sequences of the top 5 predicted off-target sites in the human genome. d, T7EI assay for the top 5 predicted off-target sites in HEK 293T cells. The lack of cleavage bands by T7EI indicates that the off-target activities of Cas9-SW1 were less than 1%. e-g, Validation of AAV-mediated Cas9-SW1 editing in vivo. e, The 10 most abundant APP_{swe} sequences from Cas9-SW1-injected 5XFAD mouse brains determined by targeted deep sequencing. Mutation sites are shown in blue text. Percentages are the frequencies of the modified APP swe sequences. Editing events are labeled with “#” f, Number of somatic mutations detected by whole-genome sequencing. The virus-transduced hippocampal regions (SW1) and untransduced brain regions (Con) of the same mouse were subjected to whole-genome sequencing to detect somatic mutations. Values are mean±SEM (n=2 mice). g, Workflow for identification of potential off-target mutations.

FIG. 9: Intrahippocampal AAV-mediated Cas9-SW1 editing in 5XFAD transgenic mice decreases amyloid plaque deposition in Cas9-expressing brain regions. a, b, Immunohistochemistry for Aβ (4G8; green), HA (red), and nuclei (blue) in 6- or 9-month-old 5XFAD mice after hippocampal injection of AAV-mediated Cas9-SW1 (EFS::SaCas9-SW1) at 3 months of age. HA staining indicates Cas9 expression. a, Representative images of sagittal sections from control (Con) and virus-injected (SW1) 5XFAD mouse brains. Scale bar: 1 mm. b, Representative images of the hippocampus, cornu ammonis (CA), and subiculum in 6- and 9-month-old mice, respectively. Scale bar: 200 μm.

FIG. 10: Intrahippocampal AAV-mediated Cas9-SW1 editing decreases gliosis in the subiculum in 5XFAD mice. a, b, Immunohistochemical analysis of amyloid plaque deposition in microglia (a) and astrocytes (b). Staining for Aβ (4G8; green) and nuclei (blue) together with Iba1 (red; a) or GFAP (red; b), respectively, in 6-month-old mice (3 months after virus injection). Scale bars: 100 μm. WT, wild type.

FIG. 11: Intrahippocampal AAV-mediated Cas9-SW1 editing decreases neurite dystrophy in 5XFAD mice. a, b, Immunohistochemistry for LAMP1 (red), Aβ (4G8; green), and nuclei (blue) in the subiculum in 5XFAD mice at 6 months (a) and 9 months (b). Scale bar: 200 μm.

FIG. 12: Intrahippocampal AAV-mediated Cas9-SW1 editing decreases amyloid plaque deposition in the Cas9-expressing brain regions in APP/PS1 transgenic mice. a, T7 endonuclease I assay for AAV-mediated Cas9-SW1 editing in the virus-transduced hippocampi from APP/PS1 mice. Arrowheads indicate the CRISPR/Cas9-edited DNA fragments. Con, Untransduced hippocampal regions of APP/PS1 mice. b, Percentage of insertion/deletion (indel) mutations. Values are mean±SEM (n=4 mice per group; *** P<0.001; unpaired two-tailed t-test). c, d, Immunohistochemistry for Aβ (4G8; green), HA (red), and nuclei (blue) in 15-month-old APP/PS1 mice (6 months after virus injection). HA staining indicates Cas9 expression. c, Representative images of sagittal sections from APP/PS1 mouse brains. Scale bar: 1 mm. d, Representative images of the hippocampus, cornu ammonis (CA), and subiculum from APP/PS1 mouse brains. Scale bar: 200 μm

FIG. 13: Optimization of the AAV construct for enhanced gene transfer and robust expression in the central nervous system. a, Schematic comparison of 2 AAV vectors for systemic delivery (with or without the insertion of the mWPRE element). ITR, inverted terminal repeat; U6, human U6 promoter; Syn, human synapsin 1 promoter; mWPRE, a truncated form of Woodchuck Hepatitis Virus (WHP) Posttranscriptional Regulatory Element (WPRE); pA, poly(A) tail. b, Immunohistochemistry for HA (green) indicates Cas9 expression in C57 mice injected with SW1-Syn::SaCas9 (−mWPRE) or SW1-Syn::SaCas9-mWPRE (+mWPRE) at a dose of 1×10¹² vg/mouse. Scale bars: 300 μm.

FIG. 14: Systemic delivery of AAV-PHP.eB-mediated Cas9-SW1 globally decreases amyloid plaque burden in 5XFAD mice. a, T7 endonuclease I assay for AAV-PHP.eB-mediated Cas9-SW1 editing in multiple brain regions in 5XFAD mice. Arrowheads indicate the CRISPR/Cas9-edited DNA fragments. HP, hippocampus; Sub, subiculum; CA, cornu ammonis; DG, dentate gyrus; CX, cortex. b, Percentage of insertion/deletion (indel) mutations. CRISPR/Cas9 editing led to the formation of indel mutations. Indel percentage indicates editing efficiency. Values are mean±SEM (n=3 mice per group). c, d, Immunohistochemistry for X34 (green), HA (red), and nuclei (blue) in 6-month-old 5XFAD mice after systemic administration of Cas9-SW1 (SW1-Syn::SaCas9-mWPRE). HA staining indicates Cas9 expression. c, Representative images of sagittal sections of 5XFAD mouse brains 3 months after virus injection. Scale bar: 1 mm. d, Representative images of the hippocampus, cornu ammonis (CA), subiculum, and dentate gyrus (DG) from 5XFAD mouse brains. Scale bars: 200 μm.

FIG. 15: Systemic delivery of AAV-PHP.eB-mediated Cas9-Con does not induce adverse effects in WT or 5XFAD mice. a-c, Systemic delivery of AAV-PHP.eB-mediated Cas9-Con did not induce adverse behavioral effects in wild-type (WT) mice. Uninject, uninjected. a, The locomotor activity of mice did not change in the exploratory open field test upon systemic delivery of AAV-PHP.eB-mediated Cas9-Con. Values are mean±SEM (n=12 mice per group; unpaired two-tailed t-test). b, Systemic delivery of AAV-PHP.eB-mediated Cas9-Con did not alter habituation in the exploratory open field (OF) test. The percentage change in the distance traveled relative to that traveled on the first day of training. Values are mean±SEM (n=12 mice per group; unpaired two-tailed t-test). c, Systemic delivery of AAV-PHP.eB-mediated Cas9-Con did not affect spatial working memory in the spontaneous alteration Y-maze test. Values are mean±SEM (n=12 mice per group; unpaired two-tailed t-test). d-f, Systemic delivery of AAV-PHP.eB-mediated Cas9-Con did not induce gliosis in WT mice. d, Immunohistochemistry for HA (red, Left panels), Iba1 (red, Middle panels), GFAP (red, Right panels), and nuclei (blue) in 6-month-old WT mice. Scale bars: 50 μm. e, Quantification of Iba1-positive cells. Values are mean±SEM (n=4 mice per group; unpaired two-tailed t-test). f, Quantification of GFAP-positive cells. Values are mean±SEM (n=4 mice per group). g, h, Immunohistochemistry for X34 (green), HA (red), and nuclei (blue) in 6-month-old 5XFAD mice after systemic administration of Cas9-Con (Con-Syn::SaCas9-mWPRE). HA tag expression indicates Cas9 expression. Scale bars: 1 mm (g), 200 μm (h). i, Quantification of X34-labeled plaque areas in the hippocampus of 6-month-old 5XFAD mice. Values are mean±SEM (n=3 mice per group; unpaired two-tailed t-test).

FIG. 16: CRISPR-targeting range of APP. The Cas9-sgRNA complex specifically disrupts the cleavage sites in exons 14 to 17 of APP. Figure is adopted from Alzforum. 3′ UTR, three prime untranslated region; AD, Alzheimer's disease; APP, amyloid precursor protein; Ex, exon.

FIG. 17: Efficient deletion of APP in HEK293T cells. a, T7 endonuclease I mismatch assay for CRISPR/Cas9-mediated genome editing in HEK293T cells carrying APP_{WT} alleles. WT1 and WT2 are single-guide RNAs (sgRNAs) that target the APP_{WT} allele. SW1 and SW2 are sgRNAs that target the APP Swedish mutation (serve as sgRNA controls; irrelevant to this invention). b, Insertion/deletion (indel) rates were examined by targeted deep sequencing. Values are mean±SEM (n=5 replicates per group; *** P<0.001; unpaired Student's t-test). APP, amyloid precursor protein; Con, control.

FIG. 18: CRISPR/Cas9-mediated deletion of β-secretase site reduces APP expression and Aβ production in HEK293T cells. Representative western blots of APP with β-actin as a loading control. HEK293T cells were co-transfected with APP-expressing construct and Cas9/gRNA constructs. L, molecular size ladder; con, control; unt, untransfected.

### DEFINITIONS

The term “APP” refers to amyloid precursor protein or the gene encoding this protein. The human APP gene is localized to human chromosome 21q11.2-q21 and encodes a protein having the exemplary amino acid sequence set forth herein as SEQ ID NO:2.

As used herein, the term “an amyloid pathology” refers to a disease or condition that is caused or exacerbated by an accumulation of excessive quantity of amyloid plaque in the pertinent tissues and anatomic sites of a person, e.g., the nervous system (such as brain), blood vessel wall, and various organs. Examples of “an amyloid pathology” include, but are not limited to, Alzheimer's disease (AD) of both the familial and sporadic types, cerebral amyloid angiopathy, Down Syndrome, as well as various amyloidosis.

The term “nucleic acid” or “polynucleotide” refers to deoxyribonucleic acids (DNA) or ribonucleic acids (RNA) and polymers thereof in either single- or double-stranded form. Unless specifically limited, the term encompasses nucleic acids containing known analogues of natural nucleotides that have similar binding properties as the reference nucleic acid and are metabolized in a manner similar to naturally occurring nucleotides. Unless otherwise indicated, a particular nucleic acid sequence also implicitly encompasses conservatively modified variants thereof (e.g., degenerate codon substitutions), alleles, orthologs, SNPs, and complementary sequences as well as the sequence explicitly indicated. Specifically, degenerate codon substitutions may be achieved by generating sequences in which the third position of one or more selected (or all) codons is substituted with mixed-base and/or deoxyinosine residues (Batzer et al., Nucleic Acid Res. 19:5081 (1991); Ohtsuka et al., J. Biol. Chem. 260:2605-2608 (1985); and Rossolini et al., Mol. Cell. Probes 8:91-98 (1994)). The term nucleic acid is used interchangeably with gene, cDNA, and mRNA encoded by a gene.

The term “gene” means the segment of DNA involved in producing a polypeptide chain. It may include regions preceding and following the coding region (leader and trailer) as well as intervening sequences (introns) between individual coding segments (exons).

The term “amino acid” refers to naturally occurring and synthetic amino acids, as well as amino acid analogs and amino acid mimetics that function in a manner similar to the naturally occurring amino acids. Naturally occurring amino acids are those encoded by the genetic code, as well as those amino acids that are later modified, e.g., hydroxyproline, Y-carboxyglutamate, and O-phosphoserine. Amino acid analogs refers to compounds that have the same basic chemical structure as a naturally occurring amino acid, i.e., an a carbon that is bound to a hydrogen, a carboxyl group, an amino group, and an R group, e.g., homoserine, norleucine, methionine sulfoxide, methionine methyl sulfonium. Such analogs have modified R groups (e.g., norleucine) or modified peptide backbones, but retain the same basic chemical structure as a naturally occurring amino acid. “Amino acid mimetics” refers to chemical compounds having a structure that is different from the general chemical structure of an amino acid, but that functions in a manner similar to a naturally occurring amino acid.

There are various known methods in the art that permit the incorporation of an unnatural amino acid derivative or analog into a polypeptide chain in a site-specific manner, see, e.g., WO 02/086075.

Amino acids may be referred to herein by either the commonly known three letter symbols or by the one-letter symbols recommended by the IUPAC-IUB Biochemical Nomenclature Commission. Nucleotides, likewise, may be referred to by their commonly accepted single-letter codes.

“Polypeptide,” “peptide,” and “protein” are used interchangeably herein to refer to a polymer of amino acid residues. All three terms apply to amino acid polymers in which one or more amino acid residue is an artificial chemical mimetic of a corresponding naturally occurring amino acid, as well as to naturally occurring amino acid polymers and non-naturally occurring amino acid polymers. As used herein, the terms encompass amino acid chains of any length, including full-length proteins, wherein the amino acid residues are linked by covalent peptide bonds.

As used herein, a “composition disrupting APP genomic sequence” refers to any composition comprising one or more agents capable of suppressing or eliminating the transcription or translation of the genomic sequence encoding the amyloid precursor protein (APP), which may be achieved by the direct deletion or alternation of at least a portion of the APP genomic sequence (e.g., by genomic editing technique such as the clustered regularly interspaced short palindromic repeat (CRISPR) system or the like) or may be achieved via reduction or elimination of the mRNA transcribed from the genomic sequence through the action of small inhibitory DNA or RNA molecules or other enzymes (e.g., antisense oligonucleotides, small inhibitory RNAs such as siRNA or shRNA, and ribozymes etc.) so as to ultimately achieve the result of reduction of APP expression up to elimination of its protein expression.

The term “targeting,” when used in the context of describing an inhibitory oligonucleotide (such as a small inhibitory RNA or an antisense oligonucleotide) or an sgRNA in relation to a genomic sequence that the inhibitory oligonucleotide or gene editing system is used to negatively regulate, refers to a sufficient sequence complementarity between at least a portion of the oligonucleotide or sgRNA and the genomic sequence, e.g., at least 80, 85, 90, 95% or higher percentage of nucleotide sequence complementarity based on the Watson-Crick base-pairing principle, so as to allow specific hybridization between the sgRNA or oligonucleotide and the genomic sequence or its mRNA transcript, which subsequently leads to the cleavage of the genomic sequence at a pre-determined location (e.g., at a location near a pre-selected mutation or a β- or γ-secretase cleavage site within the APP genomic sequence, for example, within 3, 5, 7, 8, 10, 12, 15, or 20 nucleotides upstream or downstream from the mutation or β- or γ-secretase cleavage site) or the destruction of its mRNA transcript.

The term “recombinant” when used with reference, e.g., to a cell, or a nucleic acid, protein, or vector, indicates that the cell, nucleic acid, protein or vector, has been modified by the introduction of a heterologous nucleic acid or protein or the alteration of a native nucleic acid or protein, or that the cell is derived from a cell so modified. Thus, for example, recombinant cells express genes that are not found within the native (non-recombinant) form of the cell or express native genes that are otherwise abnormally expressed, under expressed or not expressed at all.

A “promoter” is defined as an array of nucleic acid control sequences that direct transcription of a polynucleotide sequence. As used herein, a promoter includes necessary polynucleotide sequences near the start site of transcription, such as, in the case of a polymerase II type promoter, a TATA element. A promoter also optionally includes distal enhancer or repressor elements, which can be located as much as several thousand base pairs from the start site of transcription. A “constitutive” promoter is a promoter that is active under most environmental and developmental conditions. An “inducible” promoter is a promoter that is active under environmental or developmental regulation. The term “operably linked” refers to a functional linkage between a polynucleotide expression control sequence (such as a promoter, or array of transcription factor binding sites) and a second polynucleotide sequence, wherein the expression control sequence directs transcription of the polynucleotide sequence corresponding to the second sequence.

An “expression cassette” is a nucleic acid construct, generated recombinantly or synthetically, with a series of specified polynucleotide elements that permit transcription of a particular polynucleotide sequence in a host cell. An expression cassette may be part of a plasmid, viral genome, or nucleic acid fragment. Typically, an expression cassette includes a polynucleotide to be transcribed, operably linked to a promoter.

The term “heterologous” as used in the context of describing the relative location of two elements, refers to the two elements such as polynucleotide sequences (e.g., a promoter and an mRNA- or a protein/polypeptide-encoding sequence) or polypeptide sequences (e.g., two peptides as fusion partners within a fusion protein) that are not naturally found in the same relative positions. Thus, a “heterologous promoter” of a coding sequence refers to a promoter that is not naturally operably linked to that coding sequence. Similarly, a “heterologous polypeptide” or “heterologous polynucleotide” to a particular protein or its coding sequence is one derived from an origin that is different from that particular protein, or if derived from the same origin but not naturally connected to that particular protein or its coding sequence in the same fashion. The fusion of one polypeptide (or its coding sequence) with a heterologous polypeptide (or polynucleotide sequence) does not result in a longer polypeptide or polynucleotide sequence that can be found in nature.

The phrase “specifically hybridize(s) to” refers to the binding, duplexing, or hybridization of one polynucleotide sequence to another polynucleotide sequence based on Watson-Crick nucleotide base-pairing under stringent hybridization conditions when that sequences are present in a complex mixture (e.g., total cellular or library DNA or RNA). The phrase “stringent hybridization conditions” refers to conditions under which a nucleic acid (e.g., a polynucleotide probe) will hybridize to its target nucleotide sequence, typically in a complex mixture of nucleic acids, but to no other sequences. Stringent conditions are sequence-dependent and will be different in different circumstances. Longer sequences hybridize specifically at higher temperatures. An extensive guide to the hybridization of nucleic acids is found in Tijssen, Techniques in Biochemistry and Molecular Biology—Hybridization with Nucleic Probes, “Overview of principles of hybridization and the strategy of nucleic acid assays” (1993). Generally, stringent conditions are selected to be about 5-10° C. lower than the thermal melting point (Tₘ) for the specific sequence at a defined ionic strength pH. The Tₘ is the temperature (under defined ionic strength, pH, and nucleic concentration) at which 50% of the probes complementary to the target hybridize to the target sequence at equilibrium (as the target sequences are present in excess, at Tₘ, 50% of the probes are occupied at equilibrium). Stringent conditions will be those in which the salt concentration is less than about 1.0 M sodium ion, typically about 0.01 to 1.0 M sodium ion concentration (or other salts) at pH 7.0 to 8.3 and the temperature is at least about 30° C. for short probes (e.g., 10 to 50 nucleotides) and at least about 60° C. for long probes (e.g., greater than 50 nucleotides). Stringent conditions may also be achieved with the addition of destabilizing agents such as formamide. For high stringency hybridization, a positive signal is at least two times background, preferably 10 times background hybridization. Exemplary high stringency hybridization conditions include: 50% formamide, 5×SSC and 1% SDS incubated at 42° C. or 5×SSC and 1% SDS incubated at 65° C., with a wash in 0.2×SSC and 0.1% SDS at 65° C.

By “host cell” is meant a cell that contains an expression vector and supports the replication or expression of the expression vector. Host cells may be prokaryotic cells such as E. coli, or eukaryotic cells such as yeast, insect, amphibian, or mammalian cells such as CHO, HeLa and the like, e.g., cultured cells, explants, and cells in vivo.

The term “inhibiting” or “inhibition,” as used herein, refers to any detectable negative effect an inhibitory agent has on a target biological process, such as APP protein expression, formation of amyloid β (Aβ) plaques in an AD patient's brain, an AD patient's cognitive decline, protein phosphorylation, cellular signal transduction, protein synthesis, cell proliferation, tumorigenicity, and metastatic potential etc. Typically, an inhibition is reflected in a decrease of at least 10%, 20%, 30%, 40%, or 50% in target process (e.g., APP protein expression or Aβ plaque accumulation), or any one of the downstream parameters mentioned above, when compared to a control not exposed to the inhibitory agent. In a similar fashion, the term “increasing” or “increase” is used to describe any detectable positive effect an enhancing agent has on a target biological process, such as a positive change of at least 25%, 50%, 75%, 100%, or as high as 2, 3, 4, 5 or up to 10 or 20 fold, when compared to a control in the absence of the enhancer. Conversely, the term “substantially unchanged” describes a state in which the positive or negative changes are less than 10%, 5%, 2%, 1% or lower.

The term “effective amount,” as used herein, refers to an amount that is sufficient to produces an intended effect for which a substance is administered. The effect may include a desirable change in a biological process (e.g., a detectable decrease of APP expression, reduction in Aβ plaque formation, or slowing of cognitive decline in an AD patient) as well as the prevention, correction, or inhibition of progression of the symptoms of a disease/condition and related complications to any detectable extent. The exact amount “effective” for achieving a desired effect will depend on the nature of the therapeutic agent, the manner of administration, and the purpose of the treatment, and will be ascertainable by one skilled in the art using known techniques (see, e.g., Lieberman, Pharmaceutical Dosage Forms (vols. 1-3, 1992); Lloyd, The Art, Science and Technology of Pharmaceutical Compounding (1999); and Pickar, Dosage Calculations (1999)).

As used herein, the term “treatment” or “treating” includes both therapeutic and preventative measures taken to address the presence of a disease or condition or the risk of developing such disease or condition at a later time. It encompasses therapeutic or preventive measures for alleviating ongoing symptoms, inhibiting or slowing disease progression, delaying of onset of symptoms, or eliminating or reducing side-effects caused by such disease or condition. A preventive measure in this context and its variations do not require 100% elimination of the occurrence of an event; rather, they refer to a suppression or reduction in the likelihood or severity of such occurrence or a delay in such occurrence.

A “pharmaceutically acceptable” or “pharmacologically acceptable” excipient is a substance that is not biologically harmful or otherwise undesirable, i.e., the excipient may be administered to an individual along with a bioactive agent without causing any undesirable biological effects. Neither would the excipient interact in a deleterious manner with any of the components of the composition in which it is contained.

The term “excipient” refers to any essentially accessory substance that may be present in the finished dosage form of the composition of this invention. For example, the term “excipient” includes vehicles, binders, disintegrants, fillers (diluents), lubricants, glidants (flow enhancers), compression aids, colors, sweeteners, preservatives, suspending/dispersing agents, film formers/coatings, flavors and printing inks.

The term “consisting essentially of,” when used in the context of describing a composition containing an active ingredient or multiple active ingredients, refer to the fact that the composition does not contain other ingredients possessing any similar or relevant biological activity of the active ingredient(s) or capable of enhancing or suppressing the activity, whereas one or more inactive ingredients such as physiological or pharmaceutically acceptable excipients may be present in the composition. For example, a composition consisting essentially of active agent(s) effective for disrupting the APP genomic sequence or for suppressing mRNA transcribed from the genomic sequence in a subject is a composition that does not contain any other agents that may have any detectable positive or negative effect on the same target process or that may increase or decrease to any measurable extent of the disease occurrence or symptoms among the receiving subjects.

The term “about” denotes a range of +/−10% of a pre-determined value. For example, “about 10” sets a range of 90% to 110% of 10, i.e., 9 to 11.

### DETAILED DESCRIPTION OF THE INVENTION

### I. Introduction

It has long been observed and understood the correlation between abnormal amyloid plaque accumulation and various pathologies such as Alzheimer's Disease (AD). The present inventors for the first time ever achieved successful suppression of amyloid plaque build-up by using CRISPR-based genomic editing to target one or more specific sub-regions within the amyloid precursor protein (APP) genomic sequence (e.g., within close proximity to a pre-elected mutation site or a β- or γ-secretase cleavage site), while little off-target editing was detected. It is therefore demonstrated that such disruption of the APP genomic sequence provides therapeutic benefits in the treatment of patients suffering excessive build-up of amyloid plaque in the brain and other organs in a variety of pathologies in which amyloid plaque plays a critical role in the onset and progression of the disease (such as AD) as well as prophylactic benefits in the prevention or risk reduction of such disease in individuals who are not yet diagnosed of the disease but may have known risks such as family history of AD.

### II. General Recombinant Technology

Basic texts disclosing general methods and techniques in the field of recombinant genetics include Sambrook and Russell, Molecular Cloning, A Laboratory Manual (3rd ed. 2001); Kriegler, Gene Transfer and Expression: A Laboratory Manual (1990); and Ausubel et al., eds., Current Protocols in Molecular Biology (1994).

For nucleic acids, sizes are given in either kilobases (kb) or base pairs (bp). These are estimates derived from agarose or acrylamide gel electrophoresis, from sequenced nucleic acids, or from published DNA sequences. For proteins, sizes are given in kilodaltons (kDa) or amino acid residue numbers. Proteins sizes are estimated from gel electrophoresis, from sequenced proteins, from derived amino acid sequences, or from published protein sequences.

Oligonucleotides that are not commercially available can be chemically synthesized, e.g., according to the solid phase phosphoramidite triester method first described by Beaucage & Caruthers, Tetrahedron Lett. 22:1859-1862 (1981), using an automated synthesizer, as described in Van Devanter et. al., Nucleic Acids Res. 12:6159-6168 (1984). Purification of oligonucleotides is performed using any art-recognized strategy, e.g., native acrylamide gel electrophoresis or anion-exchange HPLC as described in Pearson & Reanier, J. Chrom. 255:137-149 (1983).

The sequence of a gene of interest, a polynucleotide encoding a polypeptide of interest, and synthetic oligonucleotides can be verified after cloning or subcloning using, e.g., the chain termination method for sequencing double-stranded templates of Wallace et al., Gene 16:21-26 (1981).

### II. Composition Disrupting a Genomic Sequence

Earlier work by the present inventors as well as others illustrated the involvement of APP and its mutations in the development of Alzheimer's Disease. Their latest discovery reveals that, by disrupting the APP genomic sequence especially in the close proximity of a pre-selected mutation (e.g., Swedish mutation) or a β- or γ-secretase cleavage site, APP expression and Aβ plaque accumulation in the brain can be effectively and significantly reduced. This revelation leads to the therapeutic and prophylactic use of compositions disrupting this genomic sequence, which may act at the level of intact genomic sequence at or immediately surrounding the pre-chosen the genomic locus of a specific mutation or a β- or γ-secretase cleavage site, or which may act at the level of mRNA transcribed from the genomic sequence, for treating Alzheimer's Disease in patients already diagnosed with the disease and preventing/reducing risk of later developing Alzheimer's Disease in individuals who have not yet received a diagnosis but are at heightened risk for the disease, e.g., due to family history or known genetic background (for instance, carrier of one or two APOE &4 alleles, point mutation in the genomic sequence encoding amyloid precursor protein (APP) on chromosome 21, point mutation in the genomic sequence encoding Presenilin 1 (PSEN1) on chromosome 14, and point mutation in the genomic sequence encoding Presenilin 2 (PSEN2) on chromosome 1). Various categories of possible agents acting through different mechanisms (e.g., by genomic editing or mRNA suppression) are useful in formulating such compositions for the disruption of the APP genomic sequence and are discussed below.

### A. Antisense Oligonucleotides

In some embodiments, the agent useful for disrupting the APP genomic sequence is an antisense oligonucleotide. Antisense oligonucleotides are relatively short nucleic acids that are complementary (or antisense) to the coding strand (sense strand) of the RNA transcribed from the genomic sequence encompassing the APP gene. Although antisense oligonucleotides are typically RNA-based, they can also be DNA-based. Also, antisense oligonucleotides are often modified to increase their stability.

Without being bound by theory, the binding of these relatively short oligonucleotides to the mRNA is believed to induce stretches of double-stranded RNA that trigger degradation of the messages by endogenous RNases. Additionally, sometimes the oligonucleotides are specifically designed to bind near the promoter of the coding sequence, and under these circumstances, the antisense oligonucleotides may additionally interfere with translation of the mRNA. Regardless of the specific mechanism by which antisense oligonucleotides function, their administration to a cell or tissue allows the degradation of the RNA transcribed from the genomic sequence encompassing the APP gene. Accordingly, antisense oligonucleotides decrease the expression and/or activity of encoded product from the genomic sequence.

The oligonucleotides can be DNA or RNA or chimeric mixtures or derivatives or modified versions thereof, single-stranded or double-stranded. The oligonucleotide can be modified at the base moiety, sugar moiety, or phosphate backbone, for example, to improve stability of the molecule, hybridization, etc. The oligonucleotide may include other appended groups such as peptides (e.g., for targeting host cell receptors), or agents facilitating transport across the cell membrane (see, e.g., Letsinger et al., 1989, Proc. Natl. Acad. Sci. U.S.A. 86:6553-6556; Lemaitre et al., 1987, Proc. Natl. Acad. Sci. 84:648-652; WO 88/09810) or the blood-brain barrier (see, e.g., WO 89/10134), hybridization-triggered cleavage agents (See, e.g., Krol et al., 1988, BioTechniques 6:958-976) or intercalating agents (see, e.g., Zon, 1988, Pharm. Res. 5:539-549). To this end, the oligonucleotide can be conjugated to another molecule.

Oligonucleotides of the invention may be synthesized by standard methods known in the art, e.g., by use of an automated DNA synthesizer (such as are commercially available from Biosearch, Applied Biosystems, etc.). As examples, phosphorothioate oligonucleotides may be synthesized by the method of Stein et al. (1988, Nucl. Acids Res. 16:3209), methylphosphonate oligonucleotides can be prepared by use of controlled pore glass polymer supports (Sarin et al., 1988, Proc. Natl. Acad. Sci. U.S.A. 85:7448-7451) etc.

A number of methods have been developed for delivering antisense DNA or RNA to cells, e.g., antisense molecules can be injected directly into the target anatomic site, or modified antisense molecules, designed to target the desired cells (e.g., antisense linked to peptides or antibodies that specifically bind receptors or antigens expressed on the target cell surface) can be administered systematically.

It may be difficult to achieve intracellular concentrations of the antisense sufficient to suppress translation on endogenous mRNAs in certain instances. Therefore, another approach utilizes a recombinant DNA construct in which the antisense oligonucleotide is placed under the control of a strong pol III or pol II promoter. For example, a vector can be introduced in vivo such that it is taken up by a cell and directs the transcription of an antisense RNA. Such a vector can remain episomal or become chromosomally integrated, as long as it can be transcribed to produce the desired antisense RNA. Such vectors can be constructed by recombinant DNA technology methods standard in the art. Vectors can be plasmid, viral, or others known in the art, used for replication and expression in mammalian cells. Expression of the sequence encoding the antisense RNA can be by any promoter known in the art to act in mammalian, preferably human cells. Such promoters can be inducible or constitutive. Such promoters include but are not limited to: the SV40 early promoter region (Bernoist and Chambon, 1981, Nature 290:304-310), the promoter contained in the 3′ long terminal repeat of Rous sarcoma virus (Yamamoto et al., 1980, Cell 22:787-797), the herpes thymidine kinase promoter (Wagner et al., 1981, Proc. Natl. Acad. Sci. U.S.A. 78:1441-1445), the regulatory sequences of the metallothionein gene (Brinster et al., 1982, Nature 296:39-42), etc. Any type of plasmid, cosmid, YAC or viral vector can be used to prepare the recombinant DNA construct that can be introduced directly into the target tissue site. Alternatively, viral vectors can be used which selectively infect the desired tissue, in which case administration may be accomplished by another route (e.g., systematically).

### B. Small Interfering RNA

In some embodiments, the agent is a small interfering RNA (siRNA or RNAi) molecule. RNAi constructs comprise double stranded RNA that can specifically block expression of a target gene. “RNA interference” or “RNAi” is a term initially applied to a phenomenon where double-stranded RNA (dsRNA) blocks gene expression in a specific and post-transcriptional manner. RNAi provides a useful method of inhibiting gene expression in vitro or in vivo. RNAi constructs can include small interfering RNAs (siRNAs), short hairpin RNAs (shRNAs), and other RNA species that can be cleaved in vivo to form siRNAs. RNAi constructs herein also include expression vectors (“RNAi expression vectors”) capable of giving rise to transcripts which form dsRNAs or hairpin RNAs in cells, and/or transcripts which can produce siRNAs in vivo.

RNAi expression vectors express (transcribe) RNA which produces siRNA moieties in the cell in which the construct is expressed. Such vectors include a transcriptional unit comprising an assembly of (1) genetic element(s) having a regulatory role in gene expression, for example, promoters, operators, or enhancers, operatively linked to (2) a “coding” sequence which is transcribed to produce a double-stranded RNA (two RNA moieties that anneal in the cell to form an siRNA, or a single hairpin RNA, which can be processed to an siRNA), and (3) appropriate transcription initiation and termination sequences. The choice of promoter and other regulatory elements generally varies according to the intended host cell.

The RNAi constructs contain a nucleotide sequence that hybridizes under physiologic conditions of the cell to the nucleotide sequence of at least a portion of the mRNA transcript for the gene to be inhibited (i.e., an RNA transcribed from a genomic sequence encompassing the APP gene). The double-stranded RNA need only be sufficiently similar to natural RNA that it has the ability to mediate RNAi. Thus, the invention has the advantage of being able to tolerate sequence variations that might be expected due to genetic mutation, strain polymorphism or evolutionary divergence. The number of tolerated nucleotide mismatches between the target sequence and the RNAi construct sequence is no more than 1 in 5 basepairs, or 1 in 10 basepairs, or 1 in 20 basepairs, or 1 in 50 basepairs. Mismatches in the center of the siRNA duplex are most critical and may essentially abolish cleavage of the target RNA. In contrast, nucleotides at the 3′ end of the siRNA strand that is complementary to the target RNA do not significantly contribute to specificity of the target recognition.

Production of RNAi constructs can be carried out by chemical synthetic methods or by recombinant nucleic acid techniques. Endogenous RNA polymerase of the treated cell may mediate transcription in vivo, or cloned RNA polymerase can be used for transcription in vitro. The RNAi constructs may include modifications to either the phosphate-sugar backbone or the nucleoside, e.g., to reduce susceptibility to cellular nucleases, improve bioavailability, improve formulation characteristics, and/or change other pharmacokinetic properties. For example, the phosphodiester linkages of natural RNA may be modified to include at least one of an nitrogen or sulfur heteroatom. Modifications in RNA structure may be tailored to allow specific genetic inhibition while avoiding a general response to dsRNA. Likewise, bases may be modified to block the activity of adenosine deaminase. The RNAi construct may be produced enzymatically or by partial/total organic synthesis, any modified ribonucleotide can be introduced by in vitro enzymatic or organic synthesis.

In certain embodiments, the subject RNAi constructs are “small interfering RNAs” or “siRNAs.” These nucleic acids are around 19-30 nucleotides in length, and even more preferably 21-23 nucleotides in length, e.g., corresponding in length to the fragments generated by nuclease “dicing” of longer double-stranded RNAs. The siRNAs are understood to recruit nuclease complexes and guide the complexes to the target mRNA by pairing to the specific sequences. As a result, the target mRNA is degraded by the nucleases in the protein complex. In a particular embodiment, the 21-23 nucleotides siRNA molecules comprise a 3′ hydroxyl group.

In certain embodiments, the RNAi construct is in the form of a short hairpin structure (named as shRNA). The shRNAs can be synthesized exogenously or can be formed by transcribing from RNA polymerase III promoters in vivo. Examples of making and using such hairpin RNAs for gene silencing in mammalian cells are described in, for example, Paddison et al., Genes Dev, 2002, 16:948-58; McCaffrey et al., Nature, 2002, 418:38-9; Yu et al., Proc Natl Acad Sci USA, 2002, 99:6047-52). Often, such shRNAs are engineered in cells or in an animal to ensure continuous and stable suppression of a desired gene. It is known in the art that siRNAs can be produced by processing a hairpin RNA in the cell.

A plasmid can be used to deliver the double-stranded RNA, e.g., as a transcriptional product. In such embodiments, the plasmid is designed to include a “coding sequence” for each of the sense and antisense strands of the RNAi construct. The coding sequences can be the same sequence, e.g., flanked by inverted promoters, or can be two separate sequences each under transcriptional control of separate promoters. After the coding sequence is transcribed, the complementary RNA transcripts base-pair to form the double-stranded RNA.

### C. Ribozymes

In some embodiments, the agent is a ribozyme. Ribozymes molecules designed to catalytically cleave an mRNA transcript are also used to disrupt and prevent the downstream effects of the mRNA (See, e.g., WO 90/11364; Sarver et al., 1990, Science 247:1222-1225 and U.S. Pat. No. 5,093,246). While ribozymes that cleave mRNA at site-specific recognition sequences can be used to destroy particular mRNAs, the use of hammerhead ribozymes is preferred. Hammerhead ribozymes cleave mRNAs at locations dictated by flanking regions that form complementary base pairs with the target mRNA. The sole requirement is that the target mRNA have the following sequence of two bases: 5′-UG-3′. The construction and production of hammerhead ribozymes is well known in the art and is described more fully in Haseloff and Gerlach, 1988, Nature, 334:585-591.

The ribozymes for use in this invention may also include RNA endoribonucleases (hereinafter “Cech-type ribozymes”) such as the one that occurs naturally in Tetrahymena thermophila (known as the IVS, or L-19 IVS RNA) and has been extensively described by Thomas Cech and collaborators (Zaug, et al., 1984, Science, 224:574-578; Zaug and Cech, 1986, Science, 231:470-475; Zaug, et al., 1986, Nature, 324:429-433; WO 88/04300; Been and Cech, 1986, Cell, 47:207-216). The Cech-type ribozymes have an 8-basepair active site that hybridizes to a target RNA sequence whereafter cleavage of the target RNA takes place. The invention encompasses those Cech-type ribozymes that target 8-basepair active site sequences.

As in the antisense approach, the ribozymes can be composed of modified oligonucleotides (e.g., for improved stability, targeting, etc.) and can be delivered to cells in vitro or in vivo. A preferred method of delivery involves using a DNA construct “encoding” the ribozyme under the control of a strong constitutive pol III or pol II promoter, so that transfected cells will produce sufficient quantities of the ribozyme to destroy targeted mRNA and inhibit its effect. Because ribozymes unlike antisense molecules, are catalytic, a lower intracellular concentration is required for efficiency.

There are currently two basic types of DNA enzymes, and both of these were identified by Santoro and Joyce (see, e.g., U.S. Pat. No. 6,110,462). The 10-23 DNA enzyme comprises a loop structure which connect two arms. The two arms provide specificity by recognizing the particular target nucleic acid sequence while the loop structure provides catalytic function under physiological conditions.

Briefly, to design an ideal DNA enzyme that specifically recognizes and cleaves a target nucleic acid, one of skill in the art must first identify the unique target sequence. This can be done using the same approach as outlined for antisense oligonucleotides. Preferably, the unique or substantially sequence is a G/C rich of approximately 18 to 22 nucleotides. High G/C content helps insure a stronger interaction between the DNA enzyme and the target sequence.

When synthesizing the DNA enzyme, the specific antisense recognition sequence that will target the enzyme to the message is divided so that it comprises the two arms of the DNA enzyme, and the DNA enzyme loop is placed between the two specific arms.

Methods of making and administering DNA enzymes can be found, for example, in U.S. Pat. No. 6,110,462. Similarly, methods of delivery DNA ribozymes in vitro or in vivo include methods of delivery RNA ribozyme, as outlined in detail above. Additionally, one of skill in the art will recognize that, like antisense oligonucleotide, DNA enzymes can be optionally modified to improve stability and improve resistance to degradation.

### D. Genomic Editing

The inhibition of APP expression can be achieved by way of disruption of the genetic sequence encompassing the coding sequence for this protein. One effective means of targeted gene cleavage is the CRISPR system.

The term CRISPR, abbreviation for Clustered Regularly Interspaced Short Palindromic Repeats, was originally coined in reference to segments of prokaryotic DNA that contain short, repetitive base sequences, initially found in bacteria and archaea. In a palindromic repeat, the sequence of nucleotides is the same in both directions. Each repetition is followed by short segments of spacer DNA from previous exposures to foreign DNA (e.g., DNA of a virus). Small clusters of Cas (CRISPR-associated) genes are located next to CRISPR sequences. It was later recognized that the CRISPR/Cas system is a prokaryotic immune system that confers resistance to foreign genetic elements especially those of viral origin and thereby provides a form of acquired immunity. RNA harboring the spacer sequence helps Cas (CRISPR-associated) proteins recognize and cut exogenous DNA. Other RNA-guided Cas proteins cut foreign RNA. CRISPRs are found in approximately 50% of sequenced bacterial genomes and nearly 90% of sequenced archaea, and recently the CRISPR/Cas system have been adapted for use in targeted gene editing in eukaryotic cells. See, e.g., Ledford (2016), Nature 531 (7593): 156-9.

A simple version of the CRISPR/Cas system, CRISPR/Cas9, has been modified to edit genomes. By delivering the Cas9 nuclease complexed with one or more synthetic guide RNA (gRNA) into a cell, typically by transfecting the cell with one or more expression vectors encoding for the Cas9 nuclease and the gRNA(s), the cell's genome can be cut at one or more pre-selected location, allowing a target gene (e.g., the genomic sequence harboring rs1921622) to be removed and/or substituted by a new sequence.

In the instant case, an expression vector (for example, a viral vector such as an adeno-associated virus or AAV vector) carrying the coding sequence for one or more gRNA specific for a targeted sub-region of the APP genomic sequence can be introduced into a cell in which the endogenous APP genomic sequence is to be knocked out (for example, in neuronal cells). The same expression vector optionally also carries the coding sequence for the CRISPR/Cas9 nuclease or equivalent. In the alternative, a separate expression vector may be used to introduce the CRISPR/Cas9 nuclease coding sequence for its expression in the target cells. In some cases, more than one (e.g., two) distinct gRNAs are used to ensure removal and/or replacement of a target genomic sequence (e.g., one that encompasses the APP coding sequence).

Additional gene editing systems that can be used for practicing the present invention include TALENs (Transcription activator-like effector nucleases), ZFNs (Zinc-finger nucleases), and base editing, as well as newly developed techniques such as homing endonucleases and meganucleases (MegNs) (which target and cleave DNA sequences) and prime editing (which generates RNA templates for gene alteration).

### III. Pharmaceutical Compositions and Administration

The present invention also provides pharmaceutical compositions or physiological compositions comprising an effective amount of one or more agents useful in the methods of the present invention in both prophylactic and therapeutic applications. Such pharmaceutical or physiological compositions also include one or more pharmaceutically or physiologically acceptable excipients or carriers. For instance, one exemplary composition of this invention comprises or consists essentially of one or more expression vectors encoding a CRISPR system (e.g., a Cas9 nuclease or equivalent and one or two sgRNAs) plus one or more physiologically acceptable excipients or carriers. In another exemplary composition of this invention comprises or consists essentially of one or more expression vectors encoding one or more inhibitory oligonucleotides (e.g., a small inhibitory RNA molecule or an antisense DNA or RNA oligonucleotide) plus one or more physiologically acceptable excipients or carriers. Pharmaceutical compositions of the invention are suitable for use in a variety of drug delivery systems. Suitable formulations for use in the present invention are found in Remington's Pharmaceutical Sciences, Mack Publishing Company, Philadelphia, PA, 17th ed. (1985). For a brief review of methods for drug delivery, see, Langer, Science 249:1527-1533 (1990).

The pharmaceutical compositions of the present invention can be administered by various routes, e.g., oral, subcutaneous, transdermal, intramuscular, intravenous, or intracranial. The preferred routes of administering the pharmaceutical compositions are local delivery to a relevant organ or tissue to the target disease in a recipient at a pre-determined daily dose. The appropriate dose may be administered in a single daily dose or as divided doses presented at appropriate intervals, for example as two, three, four, or more subdoses per day.

For preparing pharmaceutical compositions containing one or more active agents of this invention, inert and pharmaceutically acceptable carriers are also used. Typically, the pharmaceutical carrier can be either solid or liquid. Solid form preparations include, for example, powders, tablets, dispersible granules, capsules, cachets, and suppositories. A solid carrier can be one or more substances that can also act as diluents, flavoring agents, solubilizers, lubricants, suspending agents, binders, or tablet disintegrating agents; it can also be an encapsulating material.

In powders, the carrier is generally a finely divided solid that is in a mixture with the finely divided active component. In tablets, the active ingredient is mixed with the carrier having the necessary binding properties in suitable proportions and compacted in the shape and size desired.

For preparing pharmaceutical compositions in the form of suppositories, a low-melting wax such as a mixture of fatty acid glycerides and cocoa butter is first melted and the active ingredient is dispersed therein by, for example, stirring. The molten homogeneous mixture is then poured into convenient-sized molds and allowed to cool and solidify.

Powders and tablets preferably contain between about 5% to about 70% by weight of the active ingredient. Suitable carriers include, for example, magnesium carbonate, magnesium stearate, talc, lactose, sugar, pectin, dextrin, starch, tragacanth, methyl cellulose, sodium carboxymethyl cellulose, a low-melting wax, cocoa butter, and the like.

The pharmaceutical compositions can include the formulation of the active agent(s) with encapsulating material as a carrier providing a capsule in which the agent or agents (with or without other carriers) is/are surrounded by the carrier, such that the carrier is thus in association with the agent(s). In a similar manner, cachets can also be included. Tablets, powders, cachets, and capsules can be used as solid dosage forms suitable for oral administration.

Liquid pharmaceutical compositions include, for example, solutions suitable for oral or parenteral administration, suspensions, and emulsions suitable for oral administration. Sterile water solutions of the active component(s) or sterile solutions of the active component(s) in solvents comprising water, buffered water, saline, PBS, ethanol, or propylene glycol are examples of liquid compositions suitable for parenteral administration. The compositions may contain pharmaceutically acceptable auxiliary substances as required to approximate physiological conditions, such as pH adjusting and buffering agents, tonicity adjusting agents, wetting agents, detergents, and the like.

Sterile solutions can be prepared by dissolving the active component(s) in the desired solvent system, and then passing the resulting solution through a membrane filter to sterilize it or, alternatively, by dissolving the sterile component(s) in a previously sterilized solvent under sterile conditions. The resulting aqueous solutions may be packaged for use as is, or lyophilized, the lyophilized preparation being combined with a sterile aqueous carrier prior to administration. The pH of the preparations typically will be between 3 and 11, more preferably from 5 to 9, and most preferably from 7 to 8.

The pharmaceutical compositions containing one or more active agents can be administered for prophylactic and/or therapeutic treatments. In therapeutic applications, compositions are administered to a patient already suffering from Alzheimer's Disease in an amount sufficient to prevent, cure, reverse, or at least partially slow or arrest the symptoms of the disease and its complications, such as the onset, progression, duration, and severity of the disease. An amount adequate to accomplish this is defined as a “therapeutically effective dose.” Amounts effective for this use will depend on the severity of the disease, the weight and general state of the patient, as well as the nature of the active agent(s).

In prophylactic applications, pharmaceutical compositions containing one or more active agents are administered to a patient susceptible to or otherwise at risk of developing Alzheimer's Disease in an amount sufficient to delay or prevent the onset of the symptoms. Such an amount is defined to be a “prophylactically effective dose.” In this use, the precise amounts of the active agent(s) again depend on the patient's state of health and weight, as well as the nature of the active agent(s).

Single or multiple administrations of the compositions can be carried out with dose levels and pattern being selected by the treating physician. In any event, the pharmaceutical formulations should provide a quantity of agent(s) sufficient to effectively suppress APP expression and Aβ plaque formation in the patient, either therapeutically or prophylactically.

### IV. Therapeutic Applications Using Nucleic Acids

A variety of conditions can be treated by therapeutic approaches that involve introducing a nucleic acid encoding one or more agents disrupting APP genomic sequence or inhibiting mRNA encoded by the APP genomic sequence (such as antisense or miRNA or Cas9 nuclease and sgRNAs) into a cell such that the coding sequence is transcribed and the polypeptide or oligonucleotide agent is produced in the cell. For discussions on the application of gene therapy towards the treatment of genetic as well as acquired diseases, see, Miller Nature 357:455-460 (1992); and Mulligan Science 260:926-932 (1993).

### A. Vectors for Gene Delivery

For delivery to a cell or organism, a polynucleotide encoding one or more active agents can be incorporated into a vector. Examples of vectors used for such purposes include expression plasmids capable of directing the expression of the nucleic acids in the target cell. In other instances, the vector is a viral vector system wherein the polynucleotide is incorporated into a viral genome that is capable of transfecting the target cell. In one embodiment, the encoding polynucleotide can be operably linked to expression and control sequences that can direct expression of the polypeptide or oligonucleotide in the desired target host cells. Often, the encoding sequence is transcribed under the control of a promoter and optionally at least one transcription enhancer element. Such a promoter may be constitutive (i.e., always active for directing transcription of the coding sequence) or inducible (i.e., becoming active for directing transcription of the coding sequence upon a specific signal) in nature, and it is preferably a promoter specifically directing transcription in the particularly tissue type or cell type of the delivery target. For instance, a neuronal cell-specific promoter is preferably used for a vector intended for delivery into an anatomic site within the central nervous system, e.g., brain. Thus, one can achieve expression of the polypeptide or oligonucleotide inhibitor under appropriate conditions in the target cells.

### B. Gene Delivery Systems

Viral vector systems useful in the expression of a polypeptide or oligonucleotide disrupting a genomic sequence encompassing the APP coding sequence include, for example, naturally occurring or recombinant viral vector systems. Depending upon the particular application, suitable viral vectors include replication competent, replication deficient, and conditionally replicating viral vectors. For example, viral vectors can be derived from the genome of human or bovine adenoviruses, vaccinia virus, herpes virus, adeno-associated virus (AAV), minute virus of mice (MVM), HIV, sindbis virus, and retroviruses (including but not limited to Rous sarcoma virus and lentivirus), and MoMLV. Typically, the coding sequence of interest (e.g., one encoding for a polypeptide or oligonucleotide active agent of the present invention) are inserted into such vectors to allow packaging of the gene construct, typically with accompanying viral DNA, followed by infection of a sensitive host cell and expression of the coding sequence of interest.

As used herein, “gene delivery system” refers to any means for the delivery of a polynucleotide sequence of the interest to a target cell. In some embodiments of the invention, nucleic acids are conjugated to a cell receptor ligand for facilitated uptake (e.g., invagination of coated pits and internalization of the endosome) through an appropriate linking moiety, such as a DNA linking moiety (Wu et al., J. Biol. Chem. 263:14621-14624 (1988); WO 92/06180), or by ultrasound-microbubble delivery system (Lan H Y et al., J. Am Soc. Nephrol. 14:1535-1548). For example, nucleic acids can be linked through a polylysine moiety to asialo-oromucocid, which is a ligand for the asialoglycoprotein receptor of hepatocytes.

Similarly, viral envelopes used for packaging gene constructs that include the nucleic acids of the interest can be modified by the addition of receptor ligands or antibodies specific for a receptor to permit receptor-mediated endocytosis into specific cells (see, e.g., WO 93/20221, WO 93/14188, and WO 94/06923). In some embodiments of the invention, the DNA constructs of the invention are linked to viral proteins, such as adenovirus particles, to facilitate endocytosis (Curiel et al., Proc. Natl. Acad. Sci. U.S.A. 88:8850-8854 (1991)). In other embodiments, the active agents of the instant invention can include microtubule inhibitors (WO/9406922), synthetic peptides mimicking influenza virus hemagglutinin (Plank et al., J. Biol. Chem. 269:12918-12924 (1994)), and nuclear localization signals such as SV40 T antigen (WO93/19768).

Retroviral vectors may also be useful for introducing the coding sequence of a polypeptide or oligonucleotide active agent of the invention into target cells or tissues. Retroviral vectors are produced by genetically manipulating retroviruses. The viral genome of retroviruses is RNA. Upon infection, this genomic RNA is reverse transcribed into a DNA copy which is integrated into the chromosomal DNA of transduced cells with a high degree of stability and efficiency. The integrated DNA copy is referred to as a provirus and is inherited by daughter cells as is any other gene. The wild-type retroviral genome and the proviral DNA have three genes: the gag, the pol and the env genes, which are flanked by two long terminal repeat (LTR) sequences. The gag gene encodes the internal structural (nucleocapsid) proteins; the pol gene encodes the RNA directed DNA polymerase (reverse transcriptase); and the env gene encodes viral envelope glycoproteins. The 5′ and 3′ LTRs serve to promote transcription and polyadenylation of virion RNAs. Adjacent to the 5′ LTR are sequences necessary for reverse transcription of the genome (the tRNA primer binding site) and for efficient encapsulation of viral RNA into particles (the Psi site) (see, Mulligan, In: Experimental Manipulation of Gene Expression, Inouye (ed), 155-173 (1983); Mann et al., Cell 33:153-159 (1983); Cone and Mulligan, Proceedings of the National Academy of Sciences, U.S.A., 81:6349-6353 (1984)).

The design of retroviral vectors is well known to those of ordinary skill in the art. In brief, if the sequences necessary for encapsidation (or packaging of retroviral RNA into infectious virions) are missing from the viral genome, the result is a cis acting defect which prevents encapsidation of genomic RNA. However, the resulting mutant is still capable of directing the synthesis of all virion proteins. Retroviral genomes from which these sequences have been deleted, as well as cell lines containing the mutant genome stably integrated into the chromosome are well known in the art and are used to construct retroviral vectors. Preparation of retroviral vectors and their uses are described in many publications including, e.g., European Patent Application EPA 0 178 220; U.S. Pat. No. 4,405,712; Gilboa Biotechniques 4:504-512 (1986); Mann et al., Cell 33:153-159 (1983); Cone and Mulligan Proc. Natl. Acad. Sci. USA 81:6349-6353 (1984); Eglitis et al. Biotechniques 6:608-614 (1988); Miller et al. Biotechniques 7:981-990 (1989); Miller (1992) supra; Mulligan (1993), supra; and WO 92/07943.

The retroviral vector particles are prepared by recombinantly inserting the desired nucleotide sequence into a retrovirus vector and packaging the vector with retroviral capsid proteins by use of a packaging cell line. The resultant retroviral vector particle is incapable of replication in the host cell but is capable of integrating into the host cell genome as a proviral sequence containing the desired nucleotide sequence. As a result, the patient is capable of producing, for example, a polypeptide or polynucleotide active agent useful in the methods of the invention and thus restore the target cells (e.g., brain endothelial cells) to a normal phenotype.

Packaging cell lines that are used to prepare the retroviral vector particles are typically recombinant mammalian tissue culture cell lines that produce the necessary viral structural proteins required for packaging, but which are incapable of producing infectious virions. The defective retroviral vectors that are used, on the other hand, lack these structural genes but encode the remaining proteins necessary for packaging. To prepare a packaging cell line, one can construct an infectious clone of a desired retrovirus in which the packaging site has been deleted. Cells comprising this construct will express all structural viral proteins, but the introduced DNA will be incapable of being packaged. Alternatively, packaging cell lines can be produced by transforming a cell line with one or more expression plasmids encoding the appropriate core and envelope proteins. In these cells, the gag, pol, and env genes can be derived from the same or different retroviruses.

A number of packaging cell lines suitable for the present invention are also available in the prior art. Examples of these cell lines include Crip, GPE86, PA317 and PG13 (see Miller et al., J. Virol. 65:2220-2224 (1991)). Examples of other packaging cell lines are described in Cone and Mulligan Proceedings of the National Academy of Sciences, USA, 81:6349-6353 (1984); Danos and Mulligan Proceedings of the National Academy of Sciences, USA, 85:6460-6464 (1988); Eglitis et al. (1988), supra; and Miller (1990), supra.

Packaging cell lines capable of producing retroviral vector particles with chimeric envelope proteins may be used. Alternatively, amphotropic or xenotropic envelope proteins, such as those produced by PA317 and GPX packaging cell lines may be used to package the retroviral vectors.

### C. Pharmaceutical Formulations

When used for pharmaceutical purposes, the nucleic acid encoding a polypeptide or oligonucleotide active agent is generally formulated in a suitable buffer, which can be any pharmaceutically acceptable buffer, such as phosphate buffered saline or sodium phosphate/sodium sulfate, Tris buffer, glycine buffer, sterile water, and other buffers known to the ordinarily skilled artisan such as those described by Good et al. Biochemistry 5:467 (1966).

The compositions can additionally include a stabilizer, enhancer or other pharmaceutically acceptable carriers or vehicles. A pharmaceutically acceptable carrier can contain a physiologically acceptable compound that acts, for example, to stabilize the nucleic acids of the invention and any associated vector. A physiologically acceptable compound can include, for example, carbohydrates, such as glucose, sucrose or dextrans, antioxidants, such as ascorbic acid or glutathione, chelating agents, low molecular weight proteins or other stabilizers or excipients. Other physiologically acceptable compounds include wetting agents, emulsifying agents, dispersing agents or preservatives, which are particularly useful for preventing the growth or action of microorganisms. Various preservatives are well known and include, for example, phenol and ascorbic acid. Examples of carriers, stabilizers or adjuvants can be found in Remington's Pharmaceutical Sciences, Mack Publishing Company, Philadelphia, PA, 17th ed. (1985).

### D. Administration of Formulations

The formulations containing a polynucleotide sequence encoding a polypeptide or oligonucleotide active agent can be delivered to target tissue or organ using any delivery method known to the ordinarily skilled artisan. In some embodiments of the invention, the encoding polynucleotide sequences are formulated for subcutaneous, intramuscular, intravenous, or intraperitoneal injection, or for oral ingestion/nasal inhalation, or for topical application.

The formulations containing the nucleic acid of interest are typically directly administered to a cell. The cell can be provided as part of a tissue, such as red blood cells as a part of the circulatory system, or as an isolated cell, such as in tissue culture. The cell can be provided in vivo, ex vivo, or in vitro.

The formulations can be introduced into the tissue of interest in vivo or ex vivo by a variety of methods. In some embodiments of the invention, the nucleic acids of interest are introduced into cells by such methods as microinjection, calcium phosphate precipitation, liposome fusion, ultrasound, electroporation, or biolistics. In further embodiments, the nucleic acids are taken up directly by the target tissue or organ relevant to the disease or condition being treated, for example, when the targeted cells are the brain endothelial cells intracranial injection is appropriate.

In some embodiments of the invention, the nucleic acids of interest are administered ex vivo to cells or tissues explanted from a patient, then returned to the patient. Examples of ex vivo administration of therapeutic gene constructs include Nolta et al., Proc Natl. Acad. Sci. USA 93 (6): 2414-9 (1996); Koc et al., Seminars in Oncology 23 (1): 46-65 (1996); Raper et al., Annals of Surgery 223 (2): 116-26 (1996); Dalesandro et al., J. Thorac. Cardi. Surg., 11 (2): 416-22 (1996); and Makarov et al., Proc. Natl. Acad. Sci. USA 93 (1): 402-6 (1996).

Effective dosage of the formulations will vary depending on many different factors, including means of administration, target site, physiological state of the patient, and other medicines administered. Thus, treatment dosages will need to be titrated to optimize safety and efficacy. In determining the effective amount of the vector to be administered, the physician should evaluate the particular nucleic acid used, the disease state being diagnosed; the age, weight, and overall condition of the patient, circulating plasma levels, vector toxicities, progression of the disease, and the production of anti-vector antibodies. The size of the dose also will be determined by the existence, nature, and extent of any adverse side-effects that accompany the administration of a particular vector. For example, an antisense oligonucleotide in the amount of 1-1000, 10-200, or 20-100 mg can be delivered to a patient via intravenous injection at a frequency of weekly, bi-weekly, or monthly administration over at least one to three months or a longer time period. For CRISPR editing targeting the APP genomic region, as another example, each 5×10⁵ cells (e.g., HEK 293T cells or cells of a neuronal origin) are transfected with 0.5-50 μg; 1-20 μg; or 2-10 μg of a vector carrying genes encoding Cas9 together with a pair of sgRNAs. For CRISPR editing targeting the APP genomic region in human patients, the dose for lipid nanoparticles carrying sgRNAs and mRNA encoding Cas9 is in the range of 0.01-2; 0.02-1.0; 0.05-0.5; or 0.10-0.30 mg/kg of body weight delivered by i.v. injection 1-3 times over a period of 1-4 weeks.

### V. Kits

The invention also provides kits for treating or reducing risk of an amyloid pathology (such as Alzheimer's Disease) in a person in need thereof according to the method of the present invention. The kits typically include a container that contains (1) a pharmaceutical composition having an effective amount of one or more active agent capable of disrupting a genomic sequence encompassing APP coding sequence and/or suppressing mRNA transcribed from the genomic sequence; and (2) informational material containing instructions on how to dispense the pharmaceutical composition, including description of the type of patients who may be treated (e.g., human patients suffering from Alzheimer's Disease or at increased risk for the disease), the schedule (e.g., dose and frequency) and route of administration, and the like. In some cases, two or more containers are included in the kit to provide multiple pharmaceutical compositions each comprising an effective amount of at least one active agent, such as vector or vectors encoding components of a CRISPR system (e.g., a Cas9 nuclease or equivalent and one or more sgRNAs) or encoding an siRNA, a microRNA, a miniRNA, a lncRNA, or an antisense oligonucleotide targeting the genomic sequence encompassing APP coding sequence. Optionally, the kit may further comprise one or more additional containers, each containing at least one agent useful for sequencing at least a portion of the person's genome, especially the APP genomic sequence encompassing subregion(s) of interest, e.g., encompassing one or more sites of known mutations such as the Swedish mutation.

### EXAMPLES

The following examples are provided by way of illustration only and not by way of limitation. Those of skill in the art will readily recognize a variety of non-critical parameters that could be changed or modified to yield essentially similar results.

### Example 1: CRISPR-Mediated Disruption of APP Targeting the Swedish Mutation

Familial Alzheimer's disease is caused by dominant mutations in the genes that encode amyloid precursor protein (APP), presenilin 1, and presenilin 2. Pathological features including extracellular amyloid plaques and intracellular neurofibrillary tangles are present in multiple brain regions. Although targeting mutations using CRISPR/Cas9-mediated genome editing is a promising disease-modifying treatment strategy, brain-wide amelioration of Alzheimer's disease phenotypes using this approach has not been demonstrated. Here, we show that CRISPR/Cas9-mediated genome editing by a single administration of adeno-associated virus (AAV) efficiently and selectively disrupts mutated APP in transgenic mouse models that carry the human APP Swedish mutation, resulting in the alleviation of amyloid-beta-associated pathologies for at least 6 months post-injection. Importantly, intravenous delivery of a modified AAV-Cas9 that can cross the blood-brain barrier decreases the amyloid-beta-associated brain pathologies and the impairment of cognitive performance. Thus, our work on brain-wide gene editing provides a novel approach toward developing a noninvasive, single-administration, disease-modifying treatment for familial Alzheimer's disease and other monogenic diseases that affect multiple brain regions.

### Introduction

Alzheimer's disease (AD), one of the most prevalent neurodegenerative diseases, is characterized by the deposition of amyloid-beta (Aβ) peptides and the presence of neurofibrillary tangles composed of hyperphosphorylated tau protein. While the inherited forms of AD, termed familial AD, account for 3-5% of AD cases^{1, 2, 3}, their prevalence is comparable to that of other neurological diseases such as Huntington's disease⁴ and amyotrophic lateral sclerosis⁵. Familial AD is caused by fully penetrant and autosomal dominant mutations in the genes encoding amyloid precursor protein (APP) as well as the catalytic components of the γ-secretase complex: presenilin 1 (PS1) and presenilin 2 (PS2) 2. Dysregulated APP processing by γ-secretase causes the accumulation of Aβ peptides, which is a major factor in the disease pathogenesis⁶. Despite considerable advances in our understanding of the genetic causes of familial AD, there are currently no effective disease-modifying treatments.

Clustered regularly interspaced short palindromic repeats (CRISPR)/Cas9-mediated genome editing is a powerful tool potentially capable of targeting specific mutations⁷. However, the development of CRISPR/Cas9 as a targeted genome-editing approach for disease-modifying familial AD therapies requires addressing the needs of widespread genome editing in the brain. Like most brain disorders, AD affects multiple brain regions including the cortex and hippocampus^{8, 9}. Current genome-editing strategies for brain disorders are delivered via intraparenchymal injection¹⁰, which affects limited brain regions with restricted beneficial outcomes. Therefore, an efficient and global genome-editing method that can be used in the adult brain is urgently needed. Modified adeno-associated virus (AAV) variants that can cross the blood-brain barrier (BBB) have recently been developed, enabling widespread central nervous system transduction after intravenous delivery^{11, 12, 13, 14} Concordantly, brain-wide overexpression of a protein delivered via a BBB-crossing virus has been shown to promote recovery after ischemic stroke in aged mice¹⁴. Therefore, modifying the expression of disease-modifier gene(s) in the central nervous system by administration of a BBB-crossing virus has potential for development as a therapeutic strategy for diseases such as familial AD. However, the ability of such a virus to deliver CRISPR/Cas9 genome-editing components to ameliorate the disease phenotypes has not yet been tested in vivo.

Given that most cases of familial AD are caused by monogenic mutations, CRISPR/Cas9-mediated genome editing has been demonstrated for its ability to disrupt or correct familial AD mutations and decrease Aβ production in vitro^{15, 16}. Most patients with familial AD have a heterozygous disease-causing mutation², and humans that naturally carry one copy of APP, PSEN1, or PSEN2 do not exhibit obvious neurological symptoms^{17, 18}. Thus, disruption of the disease-causing allele in familial AD patients at the adult stage is a rational and feasible disease-modifying treatment strategy for familial AD. Among the familial APP mutations, the APP Swedish (APP_{swe}) mutation (KM670/671) is located at the sequence encoding the β-secretase cleavage site of APP and it makes the mutant APP more accessible for enzymatic cleavage, resulting in higher Aβ production^{19, 20, 21}. While a recent study reports the feasibility of disrupting the APP_{swe} mutation in APP mutation-carrying transgenic mice by genome editing, the low genome-editing efficiency of less than 3% prohibits the evaluation of the potential disease-modifying effect of disrupting this mutation¹⁶.

In the present study, we show that the AAV-mediated delivery of Cas9 and sgRNA specifically targeting the APP_{swe} mutation into the adult hippocampus generates efficient genome editing and alleviates multiple Aβ-associated pathologies in transgenic mouse models of Aβ deposition. Notably, this CRISPR-mediated beneficial effect persists for at least 6 months in the transgenic mice. Furthermore, a single systemic administration of a BBB-crossing AAV-Cas9 vector targeting the APP_{swe} mutation ameliorates Aβ pathologies throughout the brain. Thus, our results demonstrate that systemic AAV administered in a single dose is a promising approach for the development of disease-modifying therapeutic interventions for familial AD. Moreover, such a strategy could be applicable to other central nervous system disorders caused by autosomal dominant mutations that affect multiple brain regions.

### Results

### CRISPR/Cas9-Mediated Genome Editing Specifically Disrupts APP_{swe} Allele

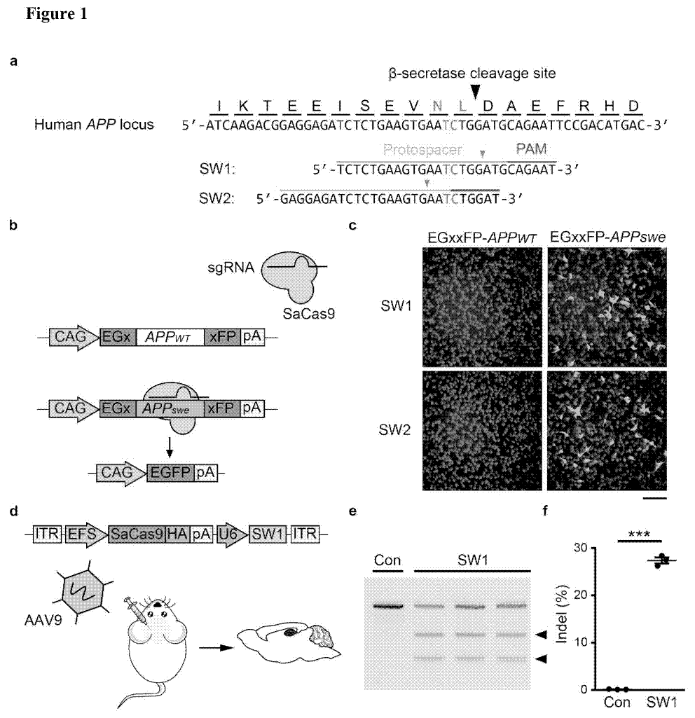

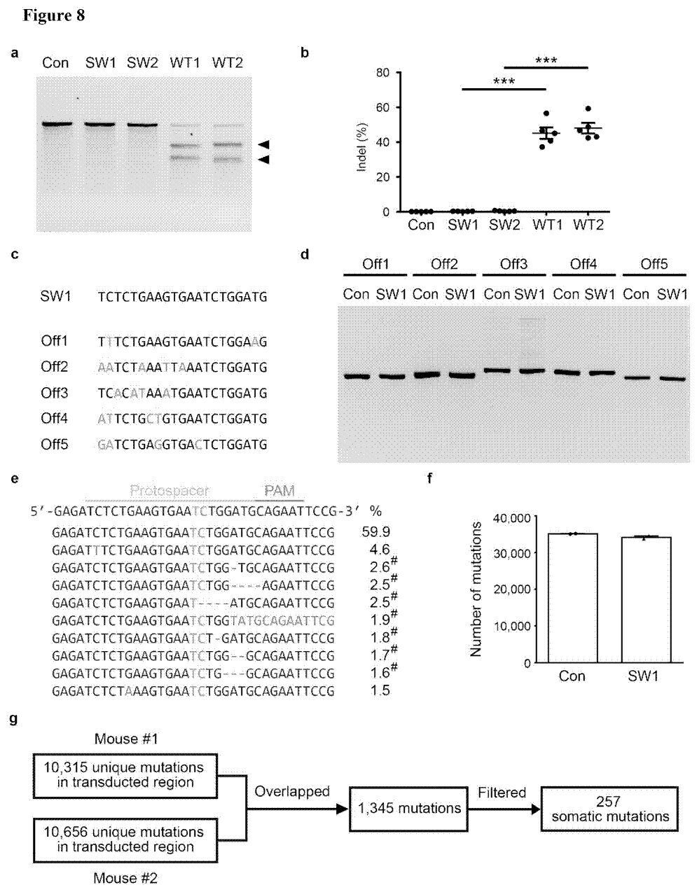

We first targeted the APP_{swe} double-base mutation (KM670/671NL) by designing 2 sgRNAs—SW1 and SW2—that place the mutation site within the first 8 nucleotides immediately adjacent to the protospacer-adjacent motif (PAM) sequence (5′-NNGRRT-3′) of Cas9 from Staphylococcus aureus (SaCas9) (FIG. 1a); this region is considered the “seed sequence” region important for Cas9-sgRNA recognition and Cas9 nuclease activity²². Because mismatches between the sgRNAs and the wild-type (WT) sequence located in the seed sequence are poorly tolerated by Cas923, we expected SW1 and SW2 to distinguish the mutant APP sequence from the WT sequence. We validated the targeting specificities of the sgRNAs by using an EGxxFP reporter system²⁴ in HEK 293T cells (FIG. 1b). The expression of enhanced green fluorescent protein (GFP) indicated that both SW1 and SW2 cleaved the APP_{swe} sequence in the EGxxFP-APP_{swe} reporter and not the APP_{WT} sequence in the EGxxFP-APP_{WT} reporter (FIG. 1c). This indicates that both SW1 and SW2 conferred high allele specificity to the APP_{swe} mutation. Given that SW1 exhibits higher genome-editing efficiency in the EGxxFP-APP_{swe} reporter and that SW1-mediated genome editing would disrupt the β-secretase cleavage site of APP, we chose SW1 for the subsequent genome-editing experiments. To confirm that the allele specificities of SW1 and SW2 were not due to their inability to edit the APP_{WT} allele, we generated 2 other sgRNAs—WT1 and WT2—to target the APP_{WT} allele at the same genetic loci corresponding to SW1 and SW2, respectively. The genome-editing efficiencies of WT1 and WT2 exceeded 50%, whereas neither SW1 nor SW2 induced any observable genome editing in HEK 293T cells carrying APP_{WT} alleles (FIG. 8a, b), indicating that SW1 and SW2 specifically targeted the APP_{swe} mutation. Moreover, examination of potential off-target effects by T7 endonuclease I mismatch assay among the top 5 computationally predicted off-target sites detected no off-target activity in HEK 293T cells (FIG. 8c, d).

To investigate the ability of the CRISPR/Cas9 system to disrupt the APP swe mutation in vivo, we packaged sgRNA SW1 together with Cas9 driven by the elongation factor 1-alpha short (EFS) promoter into a single AAV9 vector—EFS::Cas9-SW1—and injected the virus into the hippocampus of 3-month-old 5XFAD mice (FIG. 1d). These 5XFAD mice harbor human APP cDNA with K670N/M671L [Swedish], I716V [Florida], and V717I [London] mutations. The CRISPR-mediated genome editing of the APP Swedish mutation can abolish the expression of full-length mutated APP and disrupt the effects of APP mutations. Four weeks post-injection, we collected the mouse brains for genomic analysis. AAV-mediated in vivo delivery of Cas9-SW1 efficiently edited the APP_{swe} mutation in the virus-transduced brain regions (27% genome-editing efficiency; FIG. 1e, f & FIG. 8e). This suggests that AAV-mediated administration of Cas9-SW1 efficiently disrupted the APP_{swe} mutation in vivo.

We then examined the potential off-target events induced by Cas9-SW1 genome editing by performing whole-genome sequencing. A similar number of mutations (˜35,000) was detected in both the virus-transduced and untransduced regions in individual mouse brains (n=2; FIG. 8f & Table 1), suggesting that off-target events induced by Cas9-SW1 editing are rare. We subsequently identified 257 somatic mutations residing in 46 genes as potential off-target events (FIG. 8g). In particular, we identified 17 genes marked by 23 somatic mutations in exons or untranslated regions that might affect the regulation of gene expression and hence function (Table 2). Of note, the cell-type-specific transcriptome analysis suggested that only 3 genes—Sp110, Sp140, and Abcg2—were expressed in the mouse brain²⁵. Nonetheless, as these 3 genes were not prominently expressed in neurons²⁵, the identified mutations are unlikely to affect biological functions in the mouse brain.

### Cas9-SW1 Editing Decreases Amyloid Plaque Burden in 5XFAD Mice

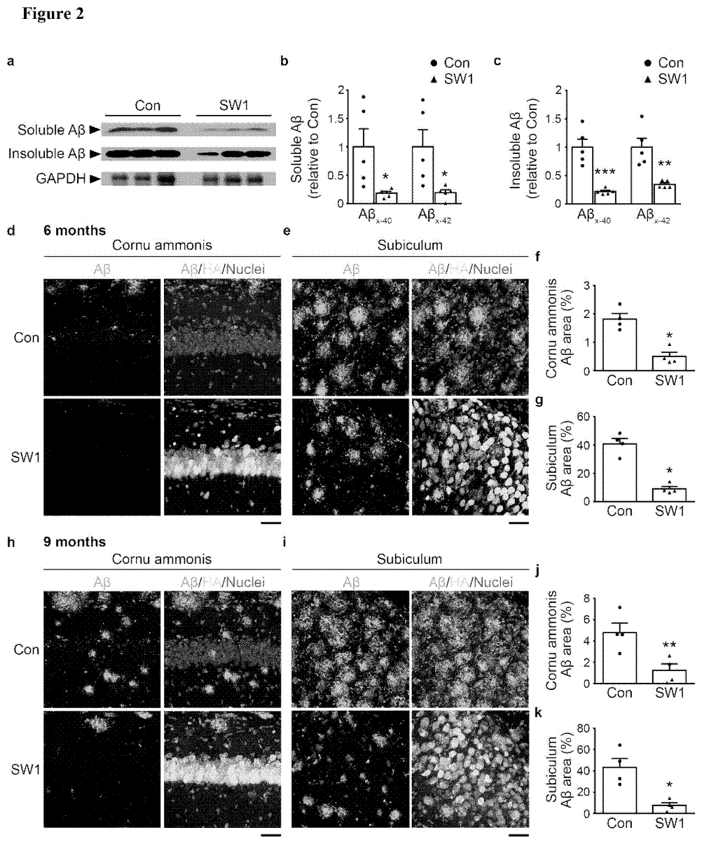

To examine whether the disruption of the APP_{swe} mutation in 5XFAD mice can decrease Aβ-associated pathologies, we delivered EFS::Cas9-SW1 via AAV injection into the hippocampus of 3-month-old 5XFAD mice, when Aβ plaques start to accumulate without causing any apparent functional deficits²⁶. By 6 months, 5XFAD mice develop severe Aβ-associated pathologies characterized by elevated Aβ levels, Aβ plaque deposition, gliosis, and neuronal dysfunction (FIG. 2-4)^{26, 27, 28, 29}. Three months after Cas9-SW1-mediated gene editing, the contents of both diethylamine-extracted soluble and formic acid-extracted insoluble Aβ in the dorsal hippocampal regions decreased (FIG. 2a) and the levels of both Aβₓ₋₄₀ and Aβₓ₋₄₂ species decreased by more than 60% (FIG. 2b, c).

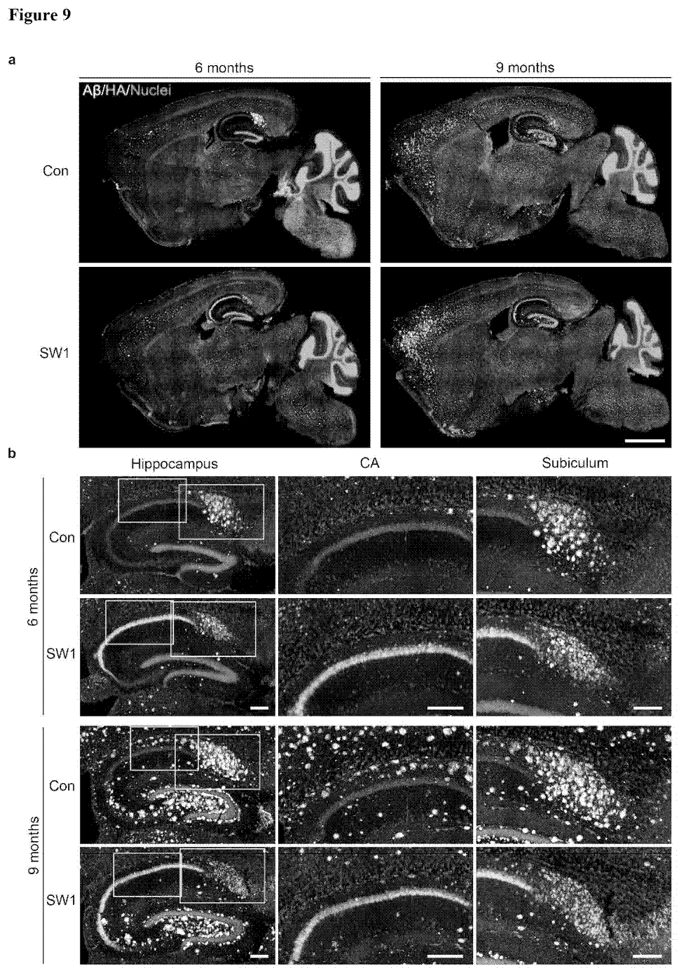

We subsequently investigated whether the AAV-mediated expression of Cas9-SW1 affects amyloid plaque deposition. Three months after injection of Cas9-SW1, 6-month-old 5XFAD mice exhibited significantly lower Aβ plaque load in the virus-transduced hippocampal subregions when compared to the corresponding subregions of the uninjected side. Upon genome editing, the Aβ-positive areas decreased by 72.9% in the cornu ammonis (CA) region (FIG. 2d, f & FIG. 9) and by 77.9% in the subiculum (FIG. 2e, g & FIG. 9). Amyloid plaque burden remained lower in 9-month-old 5XFAD mice (i.e., 6 months post-injection); the Aβ-positive area decreased by 74.3% in the CA region (FIG. 2h, j & FIG. 9) and by 83.6% in the subiculum (FIG. 2i, k & FIG. 9). These results demonstrate that the effect of Cas9-SW1-mediated editing on amyloid pathology persisted for at least 6 months after a single administration (FIG. 2h-k & FIG. 9).

### Cas9-SW1 Editing Decreases Gliosis in 5XFAD Mice

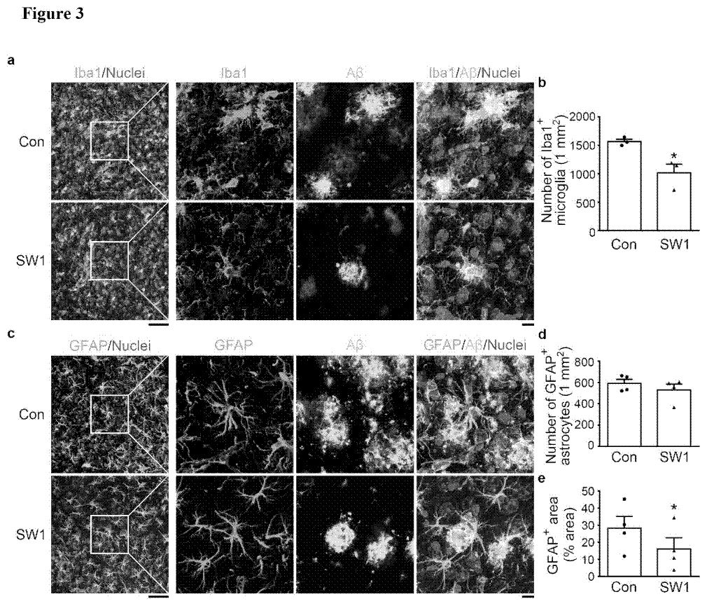

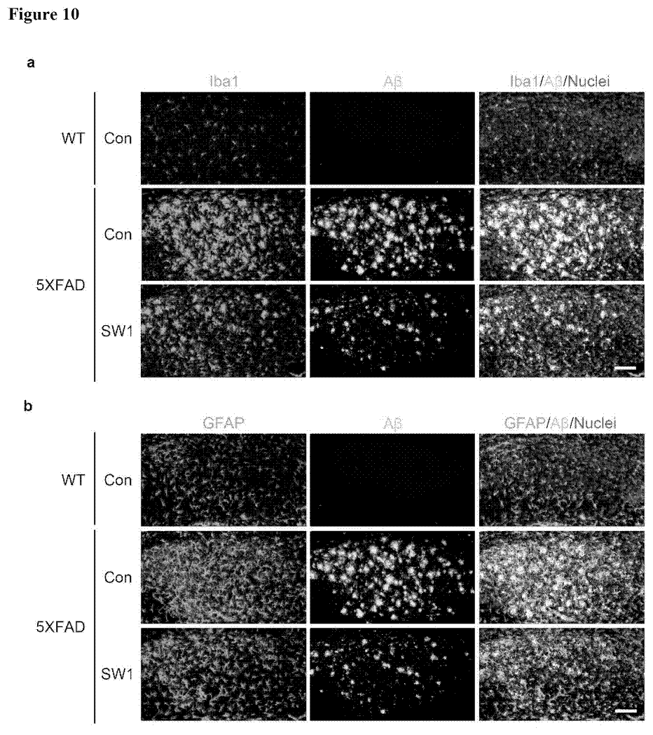

In 5XFAD mice, Aβ pathology is accompanied by gliosis, which is characterized by increased numbers of activated microglia and astrocytes surrounding amyloid plaques²⁶. Therefore, we examined whether the lower amyloid plaque burden observed in 5XFAD mice following genome editing is associated with decreased gliosis. In the subiculum of 5XFAD mice, which exhibits the most severe amyloid plaque deposition (FIG. 9), AAV-mediated Cas9-SW1 editing led to decreases of microgliosis and astrogliosis. Specifically, in 5XFAD mice, disruption of the APP_{swe} allele decreased the proportion of microglia labeled by ionized calcium-binding adapter molecule 1 (Iba1) to 64.9% (FIG. 3a, b & FIG. 10a). While AAV-mediated Cas9-SW1 editing did not change the number of astrocytes positive for glial fibrillary acidic protein (GFAP) (FIG. 3c, d & FIG. 10b), we observed a decrease in the GFAP-positive area (FIG. 3c, e & FIG. 10b). These findings collectively suggest that concurrent with the decreases in Aβ levels and deposition, CRISPR/Cas9-mediated genome editing of familial AD mutations resulted in a decrease of gliosis, as indicated by fewer microglia and decreased activation of astrocytes.

### Cas9-SW1 Editing Improves Neuronal Functions in 5XFAD Mice

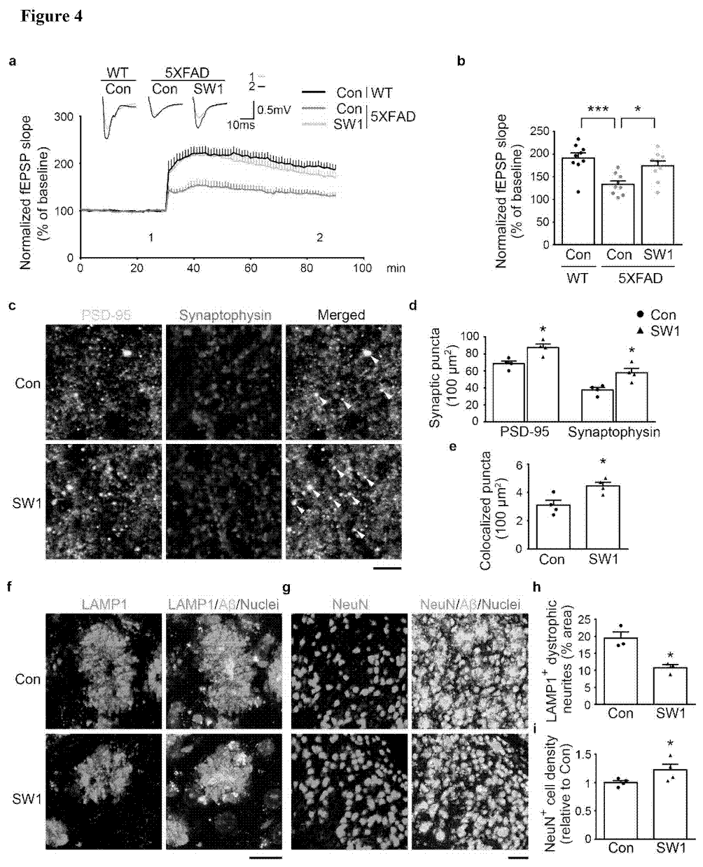

To determine whether a single injection of AAV-meditated Cas9-SW1 can reverse the hippocampal synaptic plasticity impairment in 5XFAD mice, we measured the formation of long-term potentiation (LTP). Compared to WT mice, LTP at Schaffer collateral-CA1 synapses was significantly impaired in 6-month-old 5XFAD mice; AAV-mediated Cas9-SW1 editing reversed the decrease of LTP by 90% (FIG. 4a, b). Furthermore, we demonstrated that AAV-meditated Cas9-SW1 editing increased the number of excitatory synapses in the subiculum of 5XFAD mice by 43.9%, as evidenced by co-staining for the presynaptic marker synaptophysin and postsynaptic marker postsynaptic density 95 (PSD-95) (FIG. 4c-e). These results indicate that the genome editing decreased synaptic loss in the transgenic mice.

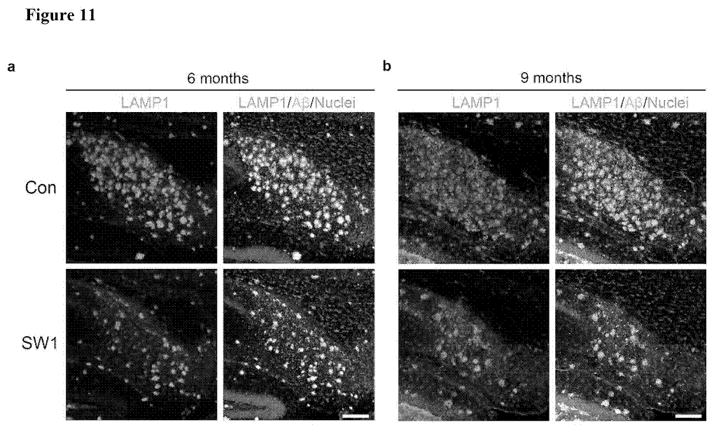

Dystrophic neurites, which are swollen neuritic processes that surround amyloid plaques, also contribute to synaptic impairment in AD. Specifically, the aggregation of lysosomes in these neurites, which can be labeled by the lysosomal protein LAMP1, alters axonal transports and synaptic release properties, consequently disrupting synaptic communication^{30, 31, 32}. Accordingly, AAV-mediated Cas9-SW1 editing in 5XFAD mice decreased LAMP1-positive dystrophic neurites by approximately 50% (FIG. 4f, h & FIG. 11) and increased the number of NeuN-positive neurons in the subiculum (FIG. 4g, i), suggesting that genome editing decreased neuronal loss.

### Cas9-SW1 Editing Decreases Aβ Pathologies in APP/PS1 Mice

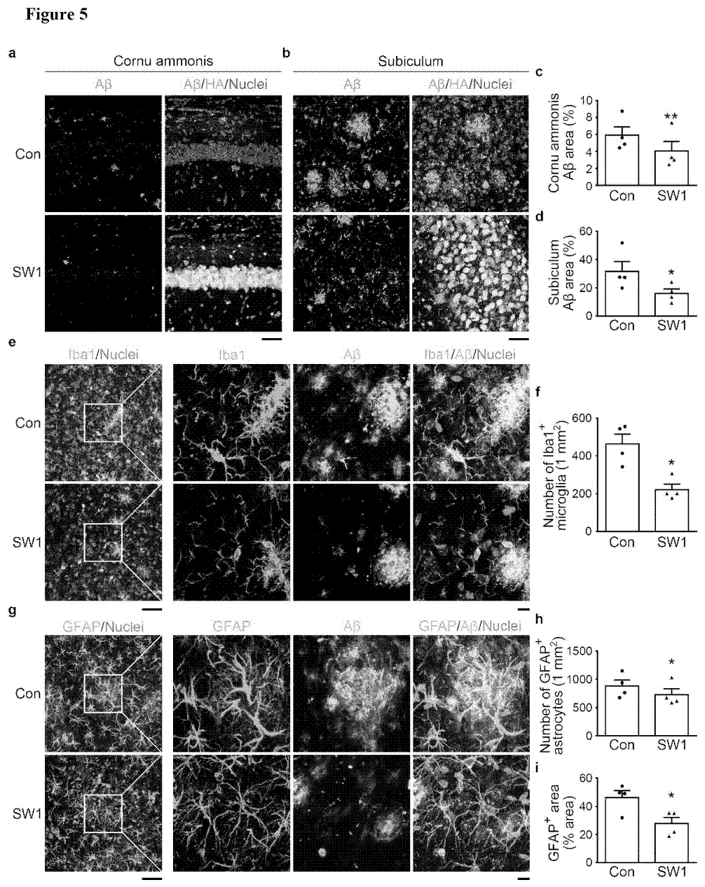

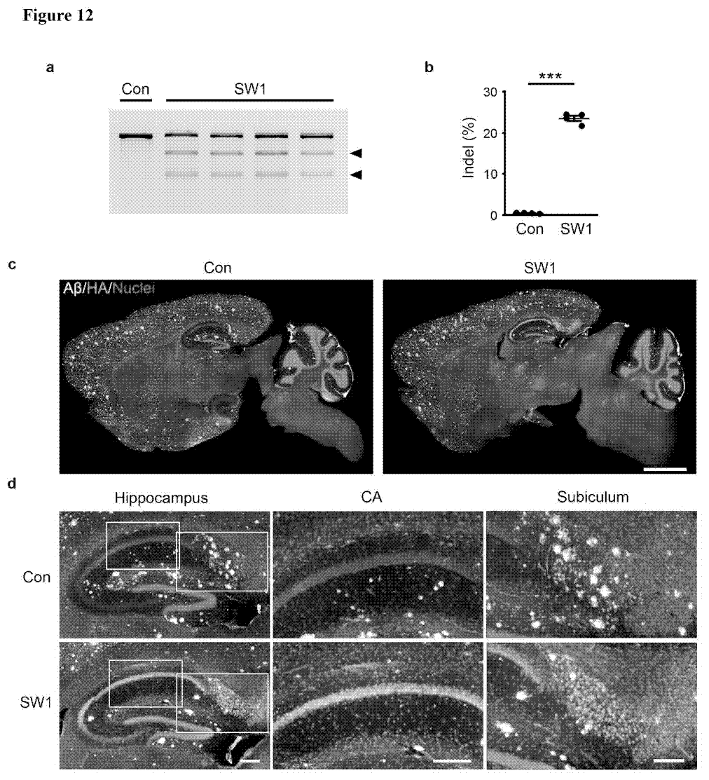

To examine the efficacy of the CRISPR-mediated disruption of the APP_{swe} mutation in transgenic mice with advanced disease status, we injected AAV-EFS::Cas9-SW1 into the hippocampus of APP/PS1 mice, a transgenic mouse model of Aβ pathology with a temporally different onset of disease phenotypes compared to 5XFAD mice. In the APP/PS1 mouse model, Aβ plaque deposition and gliosis start to appear at approximately 4 months^{33, 34}, LTP impairment and cognitive deficits are observed at 6 and 12 months, respectively^{35, 36} Accordingly, we injected AAV-EFS::Cas9-SW1 at 9 months of age (when the Aβ-associated pathologies and hippocampal synaptic dysfunctions are well developed) and collected the brains 6 months later. Genome-editing efficiency was 24% in APP/PS1 mice (FIG. 12a, b), which is comparable to 27% observed in 5XFAD mice. Moreover, after Cas9-SW1 administration, amyloid plaque burden was lower in the virus-transduced hippocampal regions of APP/PS1 mice than in the untransduced regions. Specifically, the amyloid plaque burden in the CA and subiculum in APP/PS1 mice decreased by 31.7% and 49.2%, respectively (FIG. 5a-d & FIG. 12c, d). Concurrent with the decreased Aβ deposition, Cas9-SW1 editing approximately halved the number of Iba1-labeled microglia (FIG. 5e, f). We also observed fewer GFAP-labeled astrocytes (FIG. 5g, h) and a smaller GFAP-positive area (FIG. 5g, i). These results collectively suggest that CRIPSR/Cas9-mediated genome editing decreased amyloid deposition and gliosis in transgenic mice with well-developed disease-associated phenotypes.

### Systemic Delivery of Cas9-SW1 Globally Decreases Aβ Pathologies in 5XFAD Mice

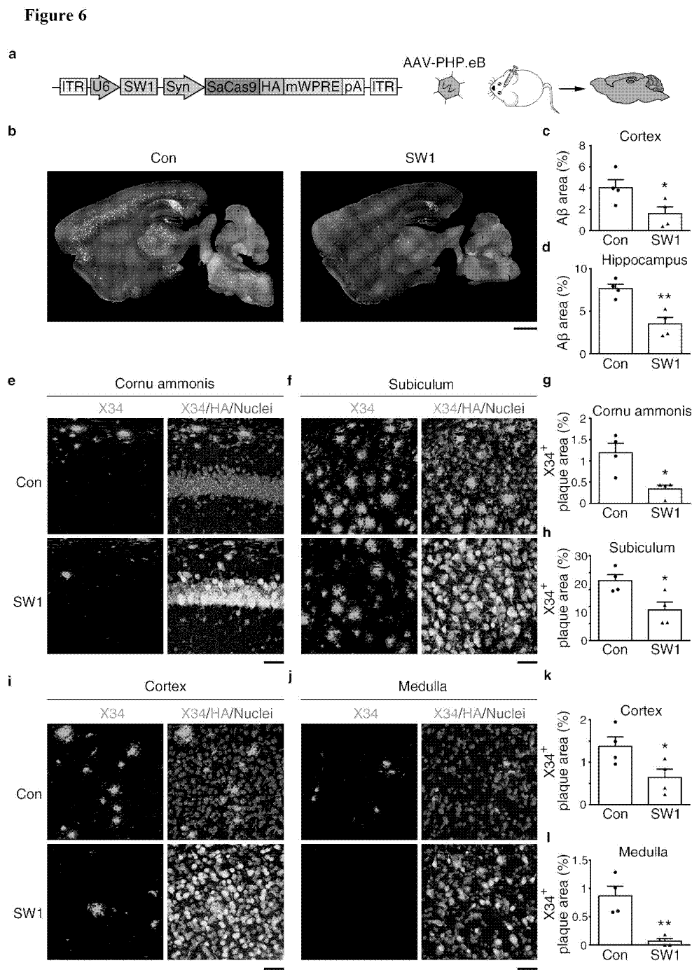

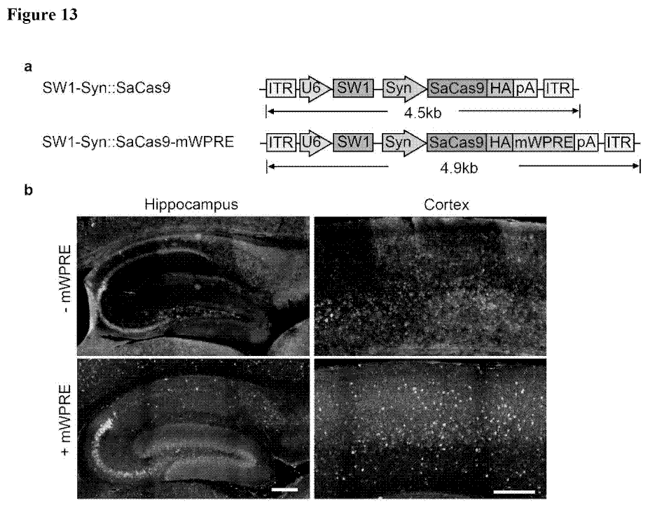

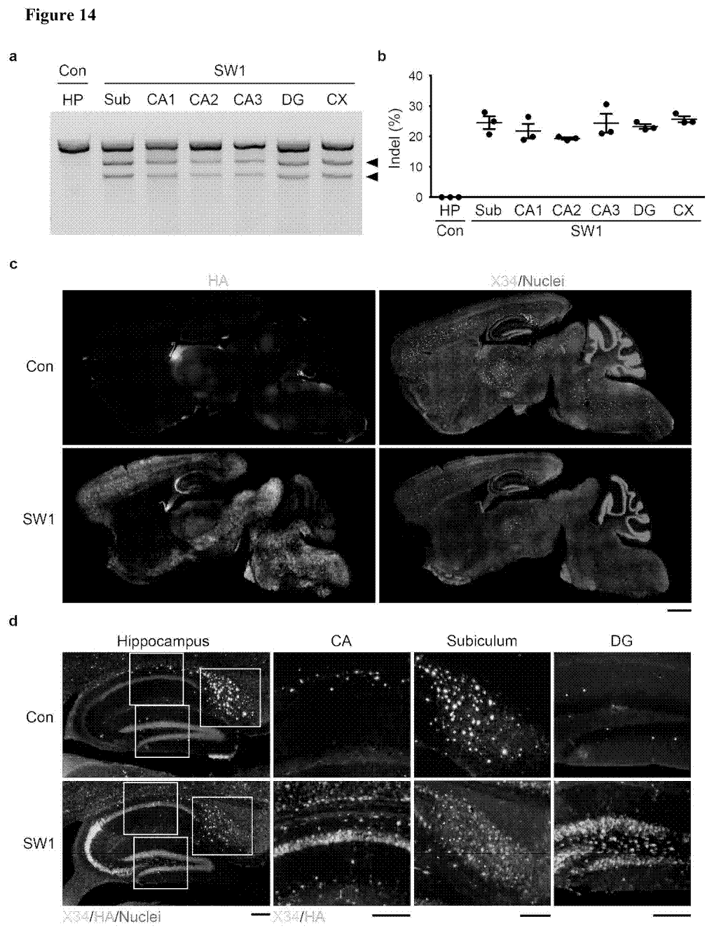

Given that hippocampal injection of AAV-EFS::Cas9-SW1 decreased Aβ-related pathologies in the virus-transduced regions (FIG. 2-4), we investigated the possibility of achieving Cas9-SW1-mediated genome editing throughout the brain. The recent development of a BBB-crossing AAV9 variant, AAV-PHP.eB¹¹, has made it possible to efficiently deliver Cas9-SW1 into the adult brain via systemic administration. Accordingly, we generated a BBB-crossing AAV-Cas9 vector driven by a neuron-specific human synapsin 1 (Syn) promoter, SW1-Syn::Cas9 (FIG. 13a). Given that systemic administration of this AAV vector only resulted in limited expression of Cas9 in the mouse brain (FIG. 13b), we generated a virus construct that would result in enhanced expression of Cas9 in vivo. Specifically, inserting a truncated form of the Woodchuck Hepatitis Virus (WHP) Posttranscriptional Regulatory Element (WPRE) 37 into the AAV construct (SW1-Syn::Cas9-mWPRE; FIG. 6a) dramatically increased Cas9 expression in the hippocampus and cortex; it also kept the size of the construct within the ˜5-kb package capacity of the AAV38 (FIG. 13a). With these optimizations, the new BBB-crossing, neuron-specific, genome-editing system achieved a genome-editing efficiency in the brain comparable to that by intrahippocampal injection (FIG. 14a, b).

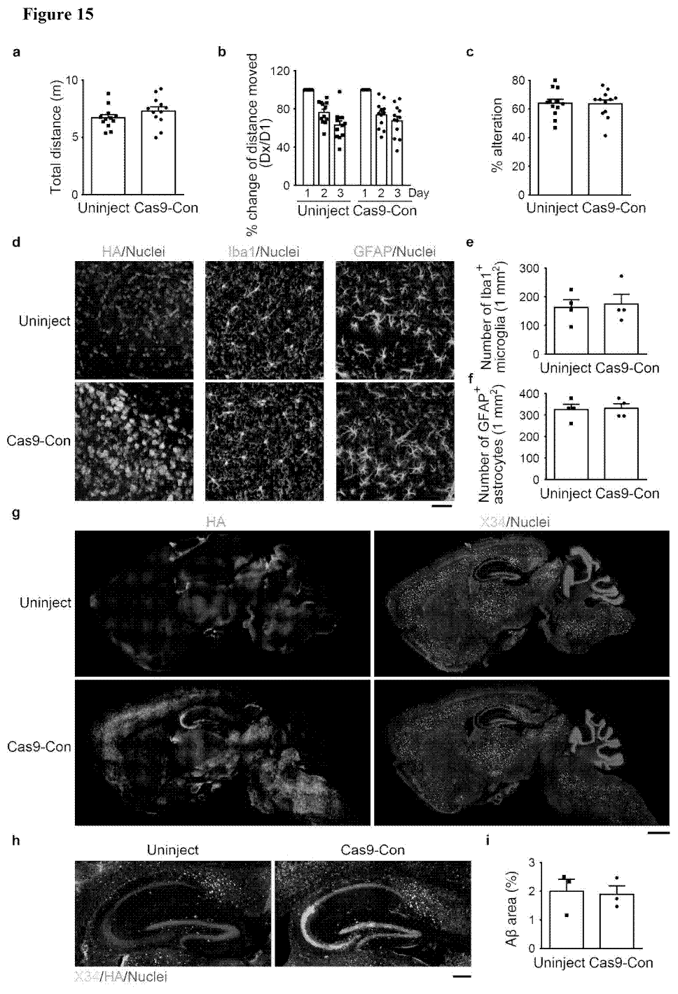

Systemic delivery of Cas9-SW1 into 5XFAD mice by AAV-PHP.eB led to widespread expression of HA-tagged Cas9 throughout the brain (FIG. 14c) and a decrease of amyloid plaque deposition in various brain regions (FIG. 6b-d). In particular, amyloid plaque burden was not only decreased in the CA and subiculum in the hippocampus (FIG. 6e-h & FIG. 14d), but also in the cortex (FIG. 6i, k) and other brain regions including the medulla of the brainstem (FIG. 6j, 1 & FIG. 14c). Meanwhile, systemic injection of Cas9 with a scrambled sgRNA (i.e., AAV-PHP.eB:Con-Syn::Cas9-mWPRE) as a control did not cause any apparent adverse effects in either WT or 5XFAD mice. Specifically, the control virus induced neither gliosis nor behavioral deficits in WT mice (FIG. 15a-f). Moreover, the control virus did not affect the amyloid plaque burden in 5XFAD mice (FIG. 15g-i). Thus, our results collectively demonstrate that systemic administration of Cas9-SW1 decreased Aβ pathologies in multiple brain regions of 5XFAD mice.

### Systemic Delivery of Cas9-SW1 Decreases Microgliosis and Neurite Dystrophy, and Improves Cognitive Performance in 5XFAD Mice

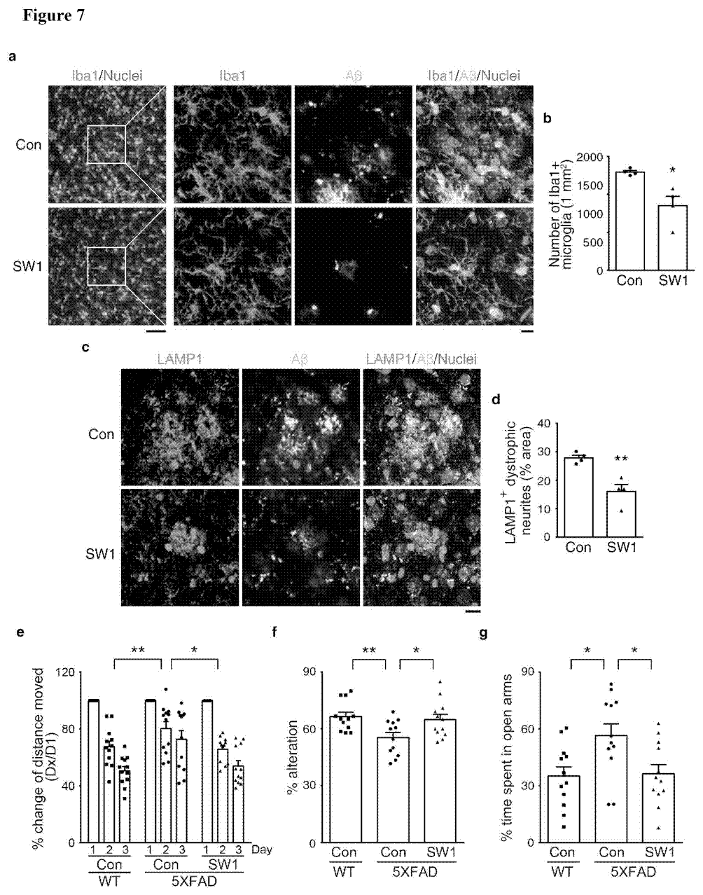

After systemic Cas9-SW1 editing, 5XFAD mice exhibited fewer Iba1-labeled microglia (FIG. 7a, b) and LAMP1-positive dystrophic neurites in the subiculum (FIG. 7d, e), suggesting that systemic delivery of Cas9-SW1 globally decreased Aβ-associated phenotypes including microgliosis and neurite dystrophy in 5XFAD mice.

Previous studies report that transgenic mouse models of Aβ deposition exhibit impaired habituation and working memory as well as increased risk-taking behavior^{26, 35, 39, 40} Given that systemic delivery of Cas9-SW1 globally decreased amyloid-associated pathologies, we assessed its beneficial effects on habituation ability, working memory, and risk-taking behavior of 5XFAD mice by using the exploratory open field test, Y-maze spontaneous alternation test, and elevated plus maze test, respectively.

In the open field test, the 5XFAD mice exhibited significantly slower habituation to the novel testing environment than the WT mice during the 3-day training course. In contrast to the uninjected 5XFAD mice, the mice that received Cas9-SW1 exhibited significantly improved habituation to the testing environment (FIG. 7e).

The Y-maze test measures the working memory of mice by allowing them to explore 3 arms of the maze. The ability of mice to alternate the arms into which they enter requires intact working memory (to remember which arm they previously visited). Compared to the WT mice, 5XFAD mice exhibited a significantly smaller percentage of spontaneous alternation among all entries (FIG. 7f). Meanwhile, 5XFAD mice that received Cas9-SW1 exhibited a significantly greater percentage of spontaneous alternation than those that did not receive Cas9-SW1 (FIG. 7f).

Meanwhile, the elevated plus maze test examines the risk-taking behavior and explorative drive of mice by evaluating their exploration of open anxiogenic areas^{39, 40}. The 5XFAD mice spent more time in the open arms than the WT mice, while the 5XFAD mice that received Cas9-SW1 spent significantly less time in the open arms, compared to uninjected 5XFAD mice (FIG. 7g). These results collectively suggest that systemic delivery of Cas9-SW1 improved the cognitive performance of the 5XFAD mice.

### Discussion

In this study, we show that a single injection of AAV-Cas9 vector selectively and efficiently edited the human APP_{swe} allele in vivo, alleviating Aβ-associated pathologies in transgenic mouse models of Aβ deposition. By utilizing an improved BBB-crossing AAV vector, AAV-PHP.eB¹¹, systemic administration of this CRISPR/Cas9 system in transgenic mice achieves efficient genome editing in multiple brain regions and exerts a global beneficial effect, ameliorating Aβ deposition, microgliosis, and neurite dystrophy as well as improving cognitive functions. To our knowledge, this is the first demonstration of systemic AAV delivery targeting a disease-causing mutation in the adult stage that resulted in efficient brain-wide genome editing, consequently alleviating disease phenotypes throughout the brain.

Among the familial AD mutations, the APP_{swe} mutation is a double-point mutation located in the “seed sequence” of SaCas9, which enables selective target recognition; this also makes it an ideal candidate for allele-specific editing, because the mismatches between the sgRNA and genomic DNA in the “seed sequence” region are poorly tolerated by Cas9 nuclease²². While selective disruption of the mutated allele of APP_{swe} has been achieved in the hippocampus in a transgenic mouse model of Aβ deposition by utilizing 2 AAVs that express an APP_{swe}-specific sgRNA and Cas9 nuclease from Streptococcus pyogenes (SpCas9, 1,368 amino acids), respectively, the genome-editing rate of that CRISPR/Cas9 system on the familial AD mutant allele was less than 3% 16. Therefore, to increase Cas9 editing efficiency, we utilized a CRISPR/Cas9 system that can be packaged into an all-in-one AAV vector with the smaller SaCas9 (1,053 amino acids) and its sgRNA instead of 2 vectors for SpCas941 Accordingly, we successfully improved the in vivo editing efficiency of the CRISPR/Cas9 system to around 30%. While the APP_{swe} mutation accounts for only a small proportion of familial AD cases, other familial AD mutations such as PSEN1 L166P are also amenable to allele-specific gene disruption. Over 200 dominant disease-causing mutations in APP, PSEN1, and PSEN2 have been identified to date⁴². Moreover, the development of the SaCas9 variant, which possesses alternative PAM compatibility⁴³, has expanded the targeting range of the CRISPR/Cas9 system, thereby broadening the candidate nucleotide sequences in familial AD mutations for allele-specific disruption. Although familial AD only accounts for approximately 3-5% of the total AD cases^{1, 2, 3}, this CRISPR/Cas9-mediated gene approach still has immense potential for therapeutic development for familial AD.

While two recent studies have demonstrated that CRISPR/Cas9-based strategies can ameliorate Aβ-associated phenotypes in transgenic mouse models, their clinical translatability might incur challenges. In one study, decreasing APP expression in the germline of APP-knock-in mice using a CRISPR/Cas9 system alleviated amyloid plaque burden⁴⁴. However, it might not be possible to translate germline editing to an intervention strategy for AD. The other study used CRISPR/Cas9-mediated genome editing to knock out β-secretase 1 (BACE1) in the hippocampus of adult transgenic mice to alleviate Aβ-associated pathologies⁴⁵. BACE1 is not a good target for clinical applications, however, because its inhibition can cause detrimental effects such as impairment of synaptic function and cognitive behaviors^{46, 47, 48}. In this study, we show that CRISPR/Cas9-mediated disruption of APP_{swe} allele ameliorates not only amyloid plaque deposition but also gliosis, synaptic dysfunctions, and cognitive impairment in transgenic mouse models of Aβ deposition. Thus, disruption of the disease-causing allele in familial AD patients in the adult stage is a promising disease-modifying strategy for familial AD.

Our study also effectively addresses one major challenge of developing CRISPR/Cas9 systems for AD therapy: the need for brain-wide genome editing. AD affects multiple brain regions, but current delivery methods into the adult brain (both viral and nonviral) require intraparenchymal injection¹⁰, which limits the Cas9-edited region. Single-site hippocampal injection of AAV9 only infects part of the hippocampus. Thus, the insufficient delivery of the CRISPR/Cas9 system may negate its potential beneficial effects. Therefore, efficient brain-wide delivery of genome-editing components is urgently needed for the therapeutic development of the CRISPR/Cas9 system for familial AD. We modified the AAV-PHP.eB11 BBB-crossing virus with the CRISPR/Cas9 system to edit the APP gene in vivo. Importantly, we used a neuron-specific promoter to drive the expression of Cas9 to achieve neuron-specific genome editing, because Aβ is mainly produced by APP-expressing neurons. This system only disrupts the mutated APP in neurons, which mitigates the potential safety issue caused by Cas9 editing, because post-mitotic neurons cannot divide. With the optimization of the Cas9 expression construct (i.e., the insertion of a truncated WPRE element into the AAV vector construct to enhance Cas9 expression), our PHP.eB-mediated CRISPR/Cas9 system achieved a global editing rate in the brain comparable to that by local intrahippocampal injection. Although AAV-PHP.eB is limited to certain strains of mice, there are emerging engineered vectors that can transduce previously resistant strains such as BALB/c and other species such as marmosets^{49, 50}. Given these ongoing developments, our work is a proof-of-concept towards genome-editing therapy using systemic delivery vectors.

To bolster the therapeutic development of CRISPR-mediated genome editing, it is important to evaluate the potential off-target events induced by genome editing. Herein, comparison of the whole-genome sequencing data of virus-transduced and untransduced brain regions within an individual animal suggested that off-target events induced by Cas9-SW1 editing are rare, as the number of the mutations in virus-transduced regions was close to the basal number in the untransduced regions. After prioritizing the mutations according to their genomic locations and the brain expression of corresponding genes, we narrowed the potential off-target events of Cas9-SW1 to 3 genes expressed in the brain—Sp110, Sp140 and Abcg2—whose expression levels might be affected. In particular, Sp110 and Sp140 belong to the nuclear body protein family, which potentially regulate gene transcription⁵¹, whereas Abcg2 belongs to the ATP-binding cassette (ABC) transporter superfamily, which serves as a xenobiotic exporter³². Notably, these genes are not prominently expressed in neurons²⁵. As the synapsin-driven Cas9 system can only target the neurons, these mutations are unlikely to affect the expression and hence functions of these genes in neurons. Therefore, the biological effects of these potential off-target mutations are likely to be minimal. Furthermore, our findings also underscore the importance of an unbiased and comprehensive analysis of potential off-target events, such as whole-genome sequencing, for the development of CRIPSR/Cas9-mediated gene therapy.

In summary, we applied a BBB-crossing, AAV-mediated, CRISPR/Cas9-based strategy to selectively and efficiently edit a disease-causing allele in vivo. Our proof-of-concept study demonstrates that the brain-wide editing of the mutated APP_{swe} allele led to persistent beneficial effects in vivo, including alleviated Aβ-associated pathologies, which makes it a promising strategy for further development. Hence, our AAV-mediated, single-dose, noninvasive CRISPR/Cas9 system represents a promising approach to address the lack of therapeutic options for familial AD as well as other forms of brain diseases caused by dominant mutations that affect multiple brain regions.

### Methods

### Molecular Cloning and GFP Reporter Assay

We adapted the AAV packaging construct from pX601-AAV-CMV::NLS-SaCas9-NLS-3×HA-bGHpA; U6::BsaI-sgRNA (Zhang Lab; Addgene plasmid #61591) 41. For intrahippocampal injection, we replaced the cytomegalovirus (CMV) promoter with the promoter of EFS. For systemic injection, we used a modified pX601 construct in which the promoter was replaced with the human synapsin 1 (hSyn) gene promoter and a truncated form of WPRE cassette (Addgene plasmid #61463) was added to enhance Cas9 expression³⁷. Moreover, we replaced bovine growth hormone (BGH) poly A with synthetic poly(A) (spA). We inserted the sgRNAs into the construct by BsaI sites. The sequences of the sgRNAs are listed in Table 3.

Masahito Ikawa generously provided the GFP reporter construct, pCAG-EGxxFP (Addgene plasmid #50716) 24, to evaluate the genome-editing efficiency of Cas9-sgRNA. We inserted the APP_{swe} and APP_{WT} alleles by ligating oligos into BamHI and EcoRI sites. We mixed 1 μg Cas9-sgRNA with 1 μg pCAG-EGxFP-APP and transfected the mixture into HEK 293T cells using Lipofectamine 3000 (Thermo Fisher Scientific). We examined the resultant GFP expression by fluorescence microscopy 12 h post-transfection.

### Mice

We obtained 5XFAD transgenic mice, which harbor 5 familial AD mutations (APP K670N/M671L [Swedish], I716V [Florida], V717I [London], and PSEN1 M146L and L286V) 26, and APP/PS1 transgenic mice, which harbor the APP K670N/M671L (Swedish) mutation and PSEN1 exon 9 deletion³³ from the Jackson Laboratory (stock no. 008730 and 004462, respectively). All experiments were conducted using male 5XFAD mice or female APP/PS1 mice. All mice were housed in the Animal and Plant Care Facility at the Hong Kong University of Science and Technology. The Animal Ethics Committee of the Hong Kong University of Science and Technology approved all animal experiments.

### Intrahippocampal and Systemic Injection of AAV

For AAV used in intrahippocampal injections, AAV9-Cas9-SW1 was generated by the Vector Core at the University of North Carolina at Chapel Hill and titered by dot blot. We anesthetized 3-month-old mice with isoflurane and injected 6×10¹⁰ vector genomes (vg) virus per animal (2 μL of 3×10¹³ vg/mL). We used the following coordinates (relative to the bregma) to target the CA1 region of the hippocampus: anteroposterior (AP), −2.0 mm; mediolateral (ML), −1.7 mm; dorsoventral (DV), −1.4 mm⁵³. Given that intrahippocampal injection of empty AAV9 vector does not generate adverse effects in mice 54, 55, 56, the uninjected side of the same mouse served as the control. We sacrificed the mice 3 or 6 months after virus injection.

For AAV used in systemic delivery, we generated AAV-PHP.eB: Cas9-Con and AAV-PHP.eB: Cas9-SW1 as previously described⁵⁷ and titered it by quantitative polymerase chain reaction (qPCR). We anesthetized 3-month-old mice and retro-orbitally injected the virus at a dose of 1× 10¹³ vg/animal (100 μL of 1× 10¹⁴ vg/mL). We sacrificed the mice at 6 or 9 months of age to analyze Aβ-associated pathologies.

### T7 Endonuclease I Assay

We performed the T7 endonuclease I mismatch assay to analyze editing efficiency. Briefly, we dissected the subiculum and cornu ammonis regions from 300-μm-thick hippocampal slices and extracted the genomic DNA using QuickExtract DNA Extraction Solution (Epicentre Biotechnologies, QE09050). Amplified DNA was denatured and reannealed from 95° C. to room temperature. We subsequently added T7 endonuclease I (New England Biolabs, M0302) and incubated the mixture at 37° C. for 15 min. We analyzed the products by 2.5% agarose gel electrophoresis for visualization. Next, we quantified the intensity of enzyme-cut and enzyme-uncut bands using ImageJ (NIH) software. Finally, we calculated the editing efficiency as follows:

$$Editing efficiency \% = 1 - \sqrt{1 - \frac{cut bands}{( uncut band + cut bands )}} \times 100$$

### Genome Sequencing and Bioinformatics Analysis

We extracted genomic DNA using the E.Z.N.A. Tissue DNA Kit (Omega Bio-tek) and subjected it to the Illumina NovaSeq (Novogene) for whole-genome sequencing (50×). We subjected raw reads (150 bp paired-end) to the Trimmomatic (version 0.32) for the trimming and filtering of low-quality reads. The reads that passed quality control were mapped to a modified version of the mouse reference genome (UCSC mm10) with the further inclusion of sequences for human APP and PSEN1 using BWA mem (version 0.7.12-r1039). Germline and somatic mutations were genotyped following the Genome Analysis Toolkit (GATK) Best Practices (Version 4.1.2.0) using the default setting. Specifically, the PCR duplicated reads were marked, and Base Quality Score Recalibration (BQSR) was carried out during the processing of BAM files. We subjected the final BAM files obtained by merging reads from multiple lanes to variant calling. We detected germline and somatic mutations using HaplotypeCaller (version 4.1.2.0) and MuTect2 (version 4.1.2.0), respectively.

We considered mutations in the virus-transduced regions of both mice that were not detected in the untransduced regions of either mouse as having greater potential to be induced by genome editing. To detect germline mutations, we applied Base Quality Score Recalibration (BQSR) using single nucleotide polymorphism (SNP) and insertion/deletion (INDEL) calls from version 3 of the Mouse Genome Project, which comprises genomic variants detected from 18 mouse strains⁵⁸. We estimated the frequencies of editing events at corresponding sites for each genomic DNA sample on the basis of the read counts of the modified and unmodified alleles stored in the VCF files. We used R programming (R studio; version 1.3.1056) to filter mutations that resided in the repetitive elements (UCSC repeatmasker mm10) and mutations that overlapped with the germline mutations. We annotated the presence of the genetic variants in the gene body from the Ensemble database obtained from the BioMart database (http://asia.ensembl.org/biomart). We further designed somatic mutations that resided in exons and untranslated regions. We subsequently examined the expression levels of associated genes in brain cells by querying the cell-type-specific transcriptome dataset (Brain RNA-Seq) 25. We considered genes to be expressed in the brain if FPKM was greater than 1 in at least one brain cell type.

### Aβ Extraction, Western Blot Analysis, and ELISA

We sequentially extracted Aβ peptides from the soluble and insoluble fractions of the dorsal hippocampi³⁵. We first homogenized frozen hippocampal tissues in tissue homogenization buffer (20 mM Tris· HCl [pH 7.4], 250 mM sucrose, 1 mM EDTA, and 1 mM EGTA) with protease inhibitor cocktail (Sigma-Aldrich). We sequentially extracted soluble and insoluble Aβ by using diethylamine and formic acid, respectively. We analyzed the levels of soluble and insoluble Aβ by western blot analysis. Moreover, we analyzed soluble Aβₓ₋₄₀ and Aβₓ₋₄₂ by using the V-PLEX Aβ Peptide Panel 1 (6E10) Kit (Meso Scale Discovery).

### Immunohistochemistry

We anesthetized the mice with pentobarbital and perfused them transcardially with Dulbecco's PBS (DPBS). We dissected the mouse brains, post-fixed them in 4% paraformaldehyde, and sectioned them into 30-μm sagittal brain slides by using a free-floating vibratome (VT1000S, Leica) for immunohistochemistry. For 4G8 immunostaining, we performed antigen retrieval with 70% formic acid. We then washed the brain sections 3 times with DPBS, blocked them with 2% goat serum and 0.3% Triton X-100 in DPBS59 for 60 min at room temperature, and then incubated them with primary antibodies overnight at 4° C.

We used the following primary antibodies: anti-HA tag antibody (1:50; #3724, Cell Signaling Technology); anti-Aβ, 17-24 antibody (1:1,000, clone 4G8, 800701, BioLegend); anti-Iba1 antibody (1:500, 019-19741, Wako); anti-GFAP antibody (1:5,000, #3670, Cell Signaling Technology); anti-PSD-95 antibody (1:500, ab2723, Abcam); anti-synaptophysin 1 antibody (1:500, 101011, Synaptic Systems); anti-LAMP-1 antibody (1:500, 1D4B, Developmental Studies Hybridoma Bank); and anti-NeuN antibody (1:100, MAβ377, Millipore).

After we incubated the sections with primary antibodies, we washed them 3 times with DPBS and 0.3% Triton X-100 (DPBST). We then incubated the sections with secondary antibodies (i.e., anti-rabbit, anti-mouse, or anti-rat Alexa Fluor 488, 546, 568, or 647; Thermo Fisher Scientific) in blocking buffer for 120 min at room temperature. After applying the corresponding secondary antibodies, we stained the brain sections with nuclear staining dye (4′,6-diamidin-2-phenylindol [DAPI, 5 μg/mL] or SYTOX Green [1:30,000, S7020, Thermo Fisher Scientific]) before mounting them onto slides.

For X34 amyloid plaque staining, we incubated the brain sections in 1 μM X34 in X34 staining buffer (40% ethanol/60% DPBS mix [pH adjusted to 10]) for 10 min at room temperature and then washed them 3 times with X34 staining buffer⁶⁰.

We performed imaging using a Leica DM6000 B compound microscope system and a Leica TCS SP8 confocal system.

### Electrophysiology

We sacrificed the mice, dissected their brains, and immediately transferred the brains to ice-cold oxygenated (95% O₂/5% CO₂) artificial cerebrospinal fluid buffer. We subsequently prepared 300-μm brain slices by using a vibratome (HM650V; Thermo Fisher Scientific) and recovered them in artificial cerebrospinal fluid at 32° C. for at least 1 h. We recorded the CA1 field excitatory postsynaptic potentials (fEPSPs) by using MED-P210A probes (Panasonic International) with a 100-μm interelectrode distance. After recording the baseline for 30 min, we induced LTP by 4 trains of theta-burst stimulation (10 brief bursts consisting of 4 pulses at 100 Hz) 61, 62. We continued to record the fEPSPs for 60 min. Finally, we quantified the magnitude of LTP as the percentage change in the average slope of the fEPSP from 50-60 min after LTP induction.

### Behavioral Tests

For animal behavioral testing, we submitted the mice to the open field test and Y-maze spontaneous alternation test, then let them rest for 2 months, and finally submitted them to the elevated plus maze test.

For the open field test, we recorded the locomotor activity of the mice using a photobeam activity system and software (San Diego Instruments) 35. We placed each mouse in the center of an open-top chamber (41× 41×38 cm) and allowed the mouse to explore the chamber for 15 min each day for 3 consecutive days. The distance moved was recorded by the photobeams.

The spontaneous alternation Y-maze test examines the spontaneous alternation performance of mice using a symmetrical Y-maze (30 cm long×20 cm high×8 cm wide) 63. Each mouse was habituated to the Y-maze environment 1 day before the test. On the test day, we placed each mouse into the center of the Y-maze and allowed it to explore for 8 min. The sequence and total number of arms entered were recorded using Etho Vision XT7 (Noldus). We calculated the percentage of alternation by dividing the number of consecutive entries into all 3 arms by the total number of arms entered minus 2. We did not count reentry into the same arm for analysis.

Finally, the elevated plus maze test measures risk-taking behavior using a plus-shaped maze elevated 60 cm above the floor. Four arms (30 cm long×5 cm wide), including 2 open arms and 2 closed arms with 15-cm-high walls, extend from the central platform. We placed each mouse on the central platform facing an open arm and allowed them to freely explore the maze for 5 min³⁹. The time the mice spent in each zone (i.e., closed arms, open arms, or the central area) was recorded using Etho Vision XT7 (Noldus).

### Statistical Analysis

All data are presented as the mean±SEM, and n-values indicate the number of individual experiments or mice. All statistical analyses were performed using GraphPad Prism (version 6.01). The significance of differences was assessed by the paired or unpaired two-tailed Student's t-test and two-way repeated-measures ANOVA as indicated in the figure legends. We used the paired two-tailed t-test to analyze the intrahippocampal injections, because the comparison was between the uninjected and injected sides of the hippocampus within a single mouse. Two-way, repeated-measures ANOVA was performed to analyze habituation in the open field test as a group comparison; the unpaired two-tailed t-test was performed in other instances. Intergroup differences were considered statistically significant at *P<0.05, ** P<0.01, and *** P<0.001. No statistical methods were used to predetermine sample size. We randomly assigned the mice to different experimental groups. The investigators who performed the immunohistochemical, electrophysiological, and the behavioral tests were blinded to the genotypes of the mice and injection conditions.

TABLE 1
Mutations detected by whole-genome sequencing in the
virus-transduced and untransduced brain regions

| | | | Shared in | Uniquely | Uniquely |
|---|---|---|---|---|---|
| | | Virus- | virus-transduced | identified in | identified in |
| | Untransduced | transduced | and untransduced | untransduced | transduced |
| Sample | region | region | regions | region (%) | region (%) |
| Mouse 1 | 35,053 | 33,727 | 23,412 | 11,641 (33.2%) | 10,315 (30.6%) |
| Mouse 2 | 35,193 | 34,458 | 23,459 | 11,734 (33.3%) | 10,656 (30.9%) |

TABLE 2
Summary of potential off-target sites and consequences

| | | Number of | Number of genes |
|---|---|---|---|
| Genomic | Number of | corresponding | expressed in |
| location | mutations | genes* | brain cells |
| Intergenic | 131 | N/A | N/A |
| Intronic | 103 | 37 | 14 |
| Exonic | 13 | 13 | 1 |
| 3′ UTR | 7 | 2 | 0 |
| 5′ UTR | 3 | 3 | 2 |

*A given gene may be counted more than once owing to the presence of multiple somatic mutations residing in different locations.

TABLE 3
Sequences of sgRNAs

| Name | Sequence | PAM |
|---|---|---|
| SW1 | TCTCTGAAGTGAATCTGGATG | CAGAAT |
| SW2 | GAGGAGATCTCTGAAGTGAAT | CTGGAT |
| WT1 | TCTCTGAAGTGAAGATGGATG | CAGAAT |
| WT2 | GAGGAGATCTCTGAAGTGAAG | ATGGAT |
| CON | GGAGACCACGGCAGGTCTCA | — |

PAM, protospacer-adjacent motif.

### Example 2: Disruption of APP Targeting the β- and γ-Secretase Cleavage Sites

### Introduction

Alzheimer's disease (AD), one of the most prevalent neurodegenerative diseases, is characterized by the deposition of amyloid-beta (Aβ) peptides and the presence of neurofibrillary tangles comprised of hyperphosphorylated Tau protein. APP (amyloid precursor protein) is the precursor protein for Aβ, one of the hallmarks of AD. Missense mutations or copy number duplication in APP gene is sufficient to cause early-onset AD. Aβ is mainly generated upon cleavage of APP in neurons; hence, decreasing the APP gene, which would lower Aβ generation, could be a potential therapeutic strategy for AD treatment. APP has a physiological role during neural development, including neuronal migration and synaptic plasticity. Thus, neuronal-specific modulation of APP in the adult stage may be a beneficial approach for treating AD with minimal adverse effects.

APP is the precursor protein of Aβ, the main constituent of amyloid plaques. APP can undergo amyloidogenic and non-amyloidogenic proteolytic pathways. In amyloidogenic pathway, APP is first cleaved by β-secretase, forming a soluble secreted form of APP (sAPPB) and a C-terminal fragment (BAPP-CTF). BAPP-CTF is sequentially cleaved by γ-secretase, resulting in the generation of the APP intracellular domain (AICD) and Aβ peptides of varying lengths, including synaptotoxic 40-residue peptide (Aβ1-40) and 42-residue peptide (Aβ1-42). The non-amyloidogenic pathway, i.e., the α-secretase-mediated cleavage pathway, inhibits generation of Aβ. In this pathway, cleavage of APP by α-secretase is within the Aβ sequence, which prevents Aβ formation.

Most familial AD-causing mutations cluster at or near the cleavage sites of α-, β-, and γ-secretases, promoting Aβ formation by favoring the proteolytic processing of APP by β- and γ-secretases. This results in increased total Aβ or Aβ42 levels and promotes the self-aggregation of Aβ into amyloid plaques. Therefore, disrupting the cleavage sites of β- and γ-secretases in APP can inhibit the amyloidogenic proteolytic pathway of APP and prevent the generation of toxic Aβ. Microbial clustered regularly interspaced short palindromic repeats (CRISPR)/Cas9-mediated genome editing is a powerful tool that disrupts the genome sequence that encodes both β- and γ-secretases and has great potential for AD treatment.

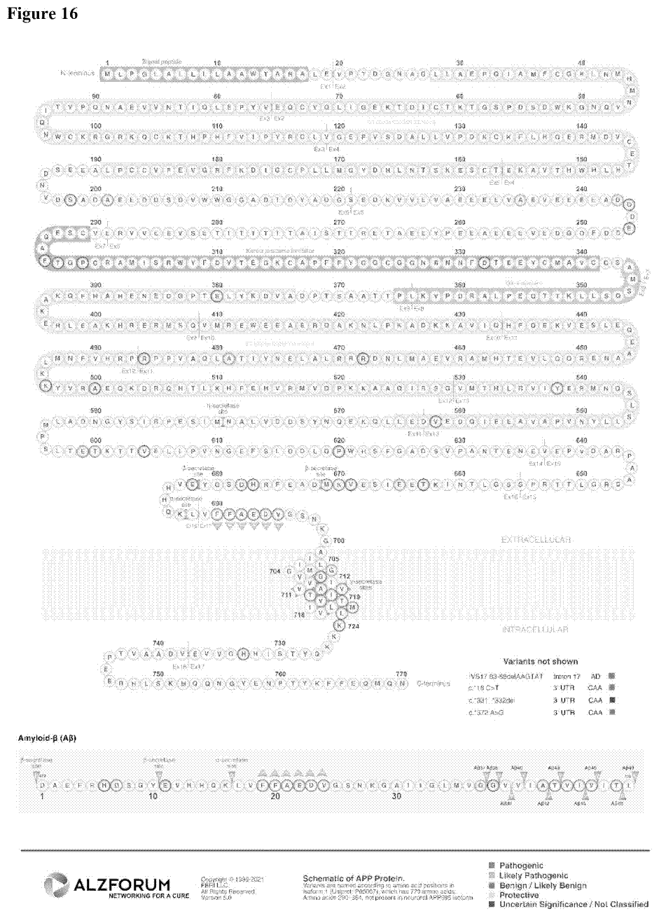

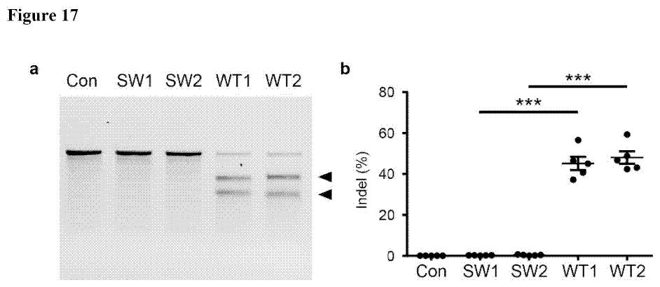

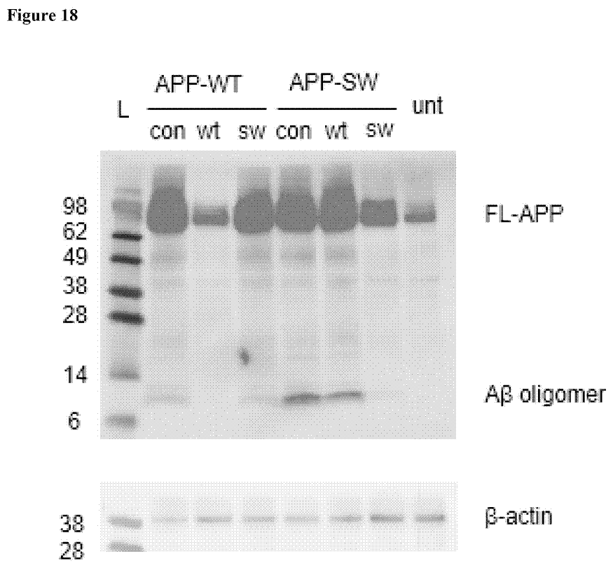

In this invention, we designed single-guide RNAs (sgRNAs) to specifically target exons 14-17 of the APP gene (FIG. 16 & Tables A, B) using a neuron-specific promoter (Table C) and analyzed the editing efficiency in vitro (FIG. 17). Compared to that targets specific mutation site (i.e., APP Swedish mutation), CRISPR/Cas9-mediated deletion of β-secretase cleavage site of APP can be used to decrease APP expression in wild-type APP as well as in APP with most of the familial APP mutations (FIG. 18, Table D). Thus, this approach can be developed for the treatment of most of familial AD cases. We demonstrated that CRISPR/Cas9-mediated deletion of β-secretase cleavage site of APP significantly reduced Aβ secretion (FIG. 18). These data collectively indicate that targeting β- and γ-secretase cleavage sites of APP is a feasible approach to reduce Aβ production and could be applied to the treatment for most of patients with familial AD mutations.

### Methods

### Plasmids

We inserted sgRNA into AAV-EFS::NLS-SaCas9-NLS-3×HA-bGHpA; U6::sgRNA via the BsaI site (New England Biolabs). We then transformed the ligation product into NEB Stable Competent E. coli (New England Biolabs) for molecular cloning. We prepared plasmid DNAs using the E.Z.N.A. Plasmid DNA Mini Kit I (Omega Bio-tek) according to the manufacturer's instructions, and prepared large-scale plasmids in endotoxin-free conditions using the Qiagen Plasmid Maxi Kit (Qiagen).

### Cell Culture

We incubated HEK 293T cells in Dulbecco's modified Eagle medium (DMEM, Invitrogen) with 10% heat-inactivated fetal bovine serum (v/v, GIBCO) plus 1% penicillin/streptomycin. We transfected HEK 293T cells using Lipofectamine 3000 Reagent (Thermo Fisher Scientific) according to the manufacturer's instructions. We harvested cells 48 h post transfection and collected medium and cell pellets.

### Genomic DNA Extraction and PCR Amplification

We extracted genomic DNA from cell pellets using QuickExtract™ DNA Extraction Solution (Lucigen) according to the manufacturer's instructions. We used KAPA HiFi HotStart ReadyMix (Roche) to amplify the genomic DNA of 293T cells that were cotransfected with the gRNA/Cas9 plasmid and pcDNA3-APP plasmid. PCR amplification was as follows: denaturation at 98° C. for 30 sec. followed by (98° C. 20 sec, 65° C. 20 sec, and 72° C. 30 sec) for 30 cycles; and 72° C. for 2 minutes as a final elongation cycle.

### T7 Endonuclease I Assay

We performed the T7 endonuclease I mismatch assay to analyze editing efficiency. We added T7 endonuclease I (New England Biolabs, M0302) into the PCR products and incubated the mixture at 37° C. for 15 min. We analyzed the products by 2.5% agarose gel electrophoresis for visualization. Next, we quantified the intensity of enzyme-cut and enzyme-uncut bands using ImageJ (NIH) software. Finally, we calculated the editing efficiency as follows:

$$Editing efficiency \% = 1 - \sqrt{1 - \frac{cut bands}{( uncut band + cut bands )}} \times 100$$

### Quantification of Secreted Aβ

We quantified secreted Aβ by using MSD Human (6E10) Abeta3-Plex Kits (Meso Scale Discovery) according to the manufacturer's instructions. We normalized secreted Aβ levels to total protein levels determined by BCA assay (Thermo Fisher Scientific).

TABLE A
Sequence of exons 14-17 in APP

| Exon | Sequence |
|---|---|
| 4 | Atgagctgcttcagaaagagcaaaactattcagatgacgtcttggccaacatgattagtgaaccaaggat |
| | cagttacggaaacgatgctctcatgccatctttgaccgaaacgaaaaccaccgtggagctccttcccgtga |
| | atggagagttcagcctggacgatctccagccgtggcattcttttggggctgactctgtgccagccaacac |
| | agaaaacgaag |
| 5 | ttgagcctgttgatgcccgccctgctgccgaccgaggactgaccactcgaccag |
| 6 | Gttctgggttgacaaatatcaagacggaggagatctctgaagtgaagatggatgcagaattccgacatg |
| | actcaggatatgaagttcatcatcaaaaattg |
| 7 | Gtgttctttgcagaagatgtgggttcaaacaaaggtgcaatcattggactcatggtgggcggtgttgtcat |
| | agcgacagtgatcgtcatcaccttggtgatgctgaagaagaaacagtacacatccattcatcatggtgtg |
| | gtggag |

TABLE B
Sequence for deletion of APP in exons 14-17

| Name | Cas9 & variants | Sequence | PAM |
|---|---|---|---|
| APP1 | SaCas9 | tcatgttggccaagacgtcat | ctgaat |
| APP2 | SaCas9 | ggccaacatgattagtgaacc | aaggat |
| APP3 | SaCas9 | accaccgtggagctccttccc | gtgaat |
| APP4 | SaCas9 | tggagctccttcccgtgaatg | gagagt |
| APP5 | SaCas9 | ctggcacagagtcagccccaa | aagaat |
| APP6 | SaCas9 | cgttttctgtgttggctggca | cagagt |
| APP7 | SaCas9 | gaggagatctctgaagtgaag | atggat |
| APP8 | SaCas9 | tctctgaagtgaagatggatg | cagaat |
| APP9 | SaCas9 | acttcatatcctgagtcatgt | cggaat |
| APP10 | SaCas9 | tttgatgatgaacttcatatc | ctgagt |
| APP11 | SaCas9 | atgcagaattccgacatgact | caggat |
| APP12 | SaCas9 | gctatgacaacaccgcccacc | atgagt |
| APP13 | SaCas9KKH | cagatgacgtcttggccaaca | aatagt |
| APP14 | SaCas9KKH | gacgtcttggccaacatgatt | tcagat |
| APP15 | SaCas9KKH | ttcagatgacgtcttggccaa | catgat |
| APP16 | SaCas9KKH | gatgacgtcttggccaacatg | attagt |
| APP17 | SaCas9KKH | gcatcgtttccgtaactgatc | cttggt |
| APP18 | SaCas9KKH | aacatgattagtgaaccaagg | atcagt |
| APP19 | SaCas9KKH | gaaccaaggatcagttacgga | aacgat |
| APP20 | SaCas9KKH | acggtggttttcgtttcggtc | aaagat |
| APP21 | SaCas9KKH | gagctccacggtggttttcgt | ttcggt |
| APP22 | SaCas9KKH | attcacgggaaggagctccac | ggtggt |
| APP23 | SaCas9KKH | tccattcacgggaaggagctc | cacggt |
| APP24 | SaCas9KKH | gtgaatggagagttcagcctg | gacgat |
| APP25 | SaCas9KKH | agccgtggcattcttttgggg | ggagat |
| APP26 | SaCas9KKH | acggaggagatctctgaagtg | cttgat |
| APP27 | SaCas9KKH | gttgacaaatatcaagacgga | ggagat |
| APP28 | SaCas9KKH | gaagtgaagatggatgcagaa | agagat |
| APP29 | SaCas9KKH | tatcaagacggaggagatctc | tgaagt |
| APP30 | SaCas9KKH | gacggaggagatctctgaagt | gaagat |
| APP31 | SaCas9KKH | attccgacatgactcaggata | tgaagt |
| APP32 | SaCas9KKH | caggatatgaagttcatcatc | aaaaat |
| APP33 | SaCas9KKH | gcagaagatgtgggttcaaac | aaaggt |
| APP34 | SaCas9KKH | agatgtgggttcaaacaaagg | tgcaat |
| APP35 | SaCas9KKH | aacaccgcccaccatgagtcc | aatgat |
| APP36 | SaCas9KKH | gacaacaccgcccaccatgag | tccaat |
| APP37 | SaCas9KKH | caaaggtgcaatcattggact | catggt |
| APP38 | SaCas9KKH | gcaatcattggactcatggtg | ggcggt |
| APP39 | SaCas9KKH | ggtgggcggtgttgtcatagc | gacagt |
| APP40 | SaCas9KKH | gggcggtgttgtcatagcgac | agtgat |
| APP41 | SaCas9KKH | cttcagcatcaccaaggtgat | gacgat |
| APP42 | SaCas9KKH | tttcttcttcagcatcaccaa | ggtgat |
| APP43 | SaCas9KKH | ctgtttcttcttcagcatcac | caaggt |
| APP44 | SaCas9KKH | agcgacagtgatcgtcatcac | cttggt |
| APP45 | SaCas9KKH | gacagtgatcgtcatcacctt | ggtgat |
| APP46 | SaCas9KKH | ccttggtgatgctgaagaaga | aacagt |
| APP47 | SaCas9KKH | aaacagtacacatccattcat | catggt |
| APP48 | SaCas9KKH | gtacacatccattcatcatgg | tgtggt |
| APP49 | CjCas9 | tttggggctgactctgtgcca | gccaacac |
| APP50 | CjCas9 | tttgaacccacatcttctgca | aagaacac |
| APP51 | CjCas9 | accgcccaccatgagtccaat | gattgcac |
| APP52 | CjCas9 | gatgacgatcactgtcgctat | gacaacac |
| APP53 | CjCas9 | ccttggtgatgctgaagaaga | aacagtac |
| APP54 | CjCas9 | ttggtgatgctgaagaagaaa | cagtacac |
| APP55 | CjCas9 | ccaccacaccatgatgaatgg | atgtgtac |

APP, amyloid precursor protein; PAM, protospacer adjacent motif.

TABLE C
Promoters for neuron-specific deletion

| Promoter | Features |
|---|---|
| Human synapsin 1 | Neuron |
| Thy1 | Neuron |
| PrP | Expression in the brain and spinal cord |
| CamKII | Excitatory neuron |
| mGAD65 | Inhibitory neuron |
| SST | Somatostatin-expressing neurons |
| TH | Tyrosine hydroxylase (TH) - expressing neurons |
| PV | Parvalbumin (PV)-expressing interneurons |

TABLE D
Mutations in APP gene

| | | Genomic | | Codon |
|---|---|---|---|---|
| Mutation | | Region | Mutation Type | Change |
| A201V | | Exon 5 | Point, Missense | GCG to GTG |
| A235V | | Exon 6 | Point, Missense | GCT to GTT |
| D243N | | Exon 6 | Point, Missense | GAT to AAT |
| E246K | | Exon 6 | Point, Missense | GAG to AAG |
| E296K | | Exon 7 | Point, Missense | GAG to AAG |
| P299L | | Exon 7 | Point, Missense | CCG to CTG |
| R468H | | Exon 11 | Point, Missense | CGC to CAC |
| A479S | | Exon 11 | Point, Missense | GCT to TCT |
| K496Q | | Exon 12 | Point, Missense | AAG to CAG |
| A500T | | Exon 12 | Point, Missense | GCA to ACA |
| Y538H | | Exon 13 | Point, Missense | TAT to CAT |
| V562I | | Exon 13 | Point, Missense | GTT to ATT |
| E599K | | Exon 14 | Point, Missense | GAA to AAA |
| T600M | | Exon 14 | Point, Missense | ACG to ATG |
| P620A | | Exon 14 | Point, Missense | CCG to GCG |
| P620L | | Exon 14 | Point, Missense | CCG to CTG |
| T663M | | Exon 16 | Point, Missense | ACG to ATG |
| E665D | | Exon 16 | Point, Missense | GAG to GAC |
| KM670/671NL | Swedish | Exon 16 | Point, Double | AAG.ATG to AAT.CTG |
| A673T | Icelandic | Exon 16 | Point, Missense | GCA to ACA |
| A673V | | Exon 16 | Point, Missense | GCA to GTA |
| H677R | English | Exon 16 | Point, Missense | CAT to CGT |
| D678H | Taiwanese | Exon 16 | Point, Missense | GAC to CAC |
| D678N | Tottori | Exon 16 | Point, Missense | GAC to AAC |
| E682K | Leuven | Exon 16 | Point, Missense | GAA to AAA |
| K687N | | Exon 16 | Point, Missense | AAA to AAT |
| A692G | Flemish | Exon 17 | Point, Missense | GCA to GGA |
| E693del | Osaka, E693Δ, | Exon 17 | Deletion | GAA to - - - |
| | E693delta | | | |
| E693G | Arctic, E22G | Exon 17 | Point, Missense | GAA to GGA |
| D694N | Iowa | Exon 17 | Point, Missense | GAT to AAT |
| G708G | | Exon 17 | Point, Silent | GGC to GGT |
| G709S | | Exon 17 | Point, Missense | GGT to AGT |
| A713T | | Exon 17 | Point, Missense | GCG to ACG |
| A713V | | Exon 17 | Point, Missense | GCG to GTG |
| T714A | Iranian | Exon 17 | Point, Missense | ACA to GCA |
| T714I | Austrian | Exon 17 | Point, Missense | ACA to ATA |
| V715A | German | Exon 17 | Point, Missense | GTG to GCG |
| V715M | French | Exon 17 | Point, Missense | GTG to ATG |
| I716F | Iberian | Exon 17 | Point, Missense | ATC to TTC |
| I716M | | Exon 17 | Point, Missense | ATC to ATG |
| I716T | | Exon 17 | Point, Missense | ATC to ACC |
| I716V | Florida | Exon 17 | Point, Missense | ATC to GTC |
| V717F | Indiana | Exon 17 | Point, Missense | GTC to TTC |
| V717G | | Exon 17 | Point, Missense | GTC to GGC |
| V717I | London | Exon 17 | Point, Missense | GTC to ATC |
| V717L | | Exon 17 | Point, Missense | GTC to CTC |
| T719P | | Exon 17 | Point, Missense | ACC to CCC |
| M722K | | Exon 17 | Point, Missense | ATG to AAG |
| L723P | Australian | Exon 17 | Point, Missense | CTG to CCG |
| K724N | Belgian | Exon 17 | Point, Missense | AAG to AAC |
| H733P | | Exon 17 | Point, Missense | CAT to CCT |
| IVS17 83-88delAAGTAT | | Exon 17 | Deletion | \ |

### Discussions

In this study, generation of amyloid-beta is first decreased by disrupting the cleavage sites in APP that involve the amyloidogenic proteolytic pathway. Abolishing the cleavage sites of β- and γ-secretases in APP (amyloid precursor protein) results in reduced generation of amyloid-beta (Aβ), one of the hallmarks of Alzheimer's disease (AD). This approach can be generally applied to all familial AD cases with mutations in APP, PSEN1 and PSEN2 and to the people with sporadic AD that are associated with increased Aβ generation.

APP expression is manipulated at the adult stage. APP has physiological roles in animal development; for example, in neuronal migration and synaptic plasticity. Thus, specific manipulation of APP expression in the brain at adult stage exhibits beneficial outcome and reduces potential side effects (due to the off-target effects of the genome-editing tool).

Neuron- or neuron subtype-specific manipulation of APP is performed. Aβ is generated from the cleavage of APP in neurons. Thus, specific manipulation of APP expression in neurons by promoter-specific-driven genome editing could avoid the potential off-target effects produced in other cell types. Furthermore, genome-editing can be conducted in specific neuronal subpopulations (i.e., inhibitory neurons, excitatory neurons) to reduce or eliminate any potential side effects.

the effects of brain-wide genome editing are studied. Noninvasive delivery of the genome-editing components are performed via intravenous administration. Whole-genome sequencing is performed and shows that Cas9-sgRNA does not introduce off-target events.

All patents, patent applications, and other publications, including GenBank Accession Numbers, cited in this application are incorporated by reference in the entirety for all purposes.

### REFERENCES

1. Cai, Y., An, S. S. & Kim, S. Mutations in presenilin 2 and its implications in Alzheimer's disease and other dementia-associated disorders. Clinical interventions in aging 10, 1163-1172 (2015).
2. Campion, D., et al. Early-onset autosomal dominant Alzheimer disease: prevalence, genetic heterogeneity, and mutation spectrum. American journal of human genetics 65, 664-670 (1999).
3. Cacace, R., Sleegers, K. & Van Broeckhoven, C. Molecular genetics of early-onset Alzheimer's disease revisited. Alzheimer's & dementia: the journal of the Alzheimer's Association 12, 733-748 (2016).
4. Harper, P. S. The epidemiology of Huntington's disease. Human genetics 89, 365-376 (1992).
5. Chio, A., et al. Global epidemiology of amyotrophic lateral sclerosis: a systematic review of the published literature. Neuroepidemiology 41, 118-130 (2013).
6. Selkoe, D. J. & Hardy, J. The amyloid hypothesis of Alzheimer's disease at 25 years. EMBO molecular medicine 8, 595-608 (2016).
7. Ran, F. A., et al. Genome engineering using the CRISPR-Cas9 system. Nature protocols 8, 2281-2308 (2013).
8. Serrano-Pozo, A., Frosch, M. P., Masliah, E. & Hyman, B. T. Neuropathological alterations in Alzheimer disease. Cold Spring Harbor perspectives in medicine 1, a006189 (2011).
9. Braak, H. & Braak, E. Frequency of stages of Alzheimer-related lesions in different age categories. Neurobiology of aging 18, 351-357 (1997).
10. Ingusci, S., Verlengia, G., Soukupova, M., Zucchini, S. & Simonato, M. Gene Therapy Tools for Brain Diseases. Frontiers in pharmacology 10, 724 (2019).
11. Chan, K. Y., et al. Engineered AAVs for efficient noninvasive gene delivery to the central and peripheral nervous systems. Nature neuroscience 20, 1172-1179 (2017).
12. Deverman, B. E., et al. Cre-dependent selection yields AAV variants for widespread gene transfer to the adult brain. Nature biotechnology 34, 204-209 (2016).
13. Bedbrook, C. N., Deverman, B. E. & Gradinaru, V. Viral Strategies for Targeting the Central and Peripheral Nervous Systems. Annual review of neuroscience 41, 323-348 (2018).
14. Zeng, J., et al. TRIM9-Mediated Resolution of Neuroinflammation Confers Neuroprotection upon Ischemic Stroke in Mice. Cell reports 27, 549-560 e546 (2019).
15. Ortiz-Virumbrales, M., et al. CRISPR/Cas9-Correctable mutation-related molecular and physiological phenotypes in iPSC-derived Alzheimer's PSEN2 (N141I) neurons. Acta neuropathologica communications 5, 77 (2017).
16. Gyorgy, B., et al. CRISPR/Cas9 Mediated Disruption of the Swedish APP Allele as a Therapeutic Approach for Early-Onset Alzheimer's Disease. Molecular therapy. Nucleic acids 11, 429-440 (2018).
17. Karczewski, K. J., et al. The mutational constraint spectrum quantified from variation in 141,456 humans. Nature 581, 434-443 (2020).
18. Wang, B., et al. Gamma-secretase gene mutations in familial acne inversa. Science 330, 1065 (2010).
19. Mullan, M., et al. A pathogenic mutation for probable Alzheimer's disease in the APP gene at the N-terminus of beta-amyloid. Nature genetics 1, 345-347 (1992).
20. Citron, M., et al. Excessive production of amyloid beta-protein by peripheral cells of symptomatic and presymptomatic patients carrying the Swedish familial Alzheimer disease mutation. Proceedings of the National Academy of Sciences of the United States of America 91, 11993-11997 (1994).
21. Johnston, J. A., et al. Increased beta-amyloid release and levels of amyloid precursor protein (APP) in fibroblast cell lines from family members with the Swedish Alzheimer's disease APP670/671 mutation. FEBS letters 354, 274-278 (1994).
22. Wiedenheft, B., et al. RNA-guided complex from a bacterial immune system enhances target recognition through seed sequence interactions. Proceedings of the National Academy of Sciences of the United States of America 108, 10092-10097 (2011).
23. Gao, X., et al. Treatment of autosomal dominant hearing loss by in vivo delivery of genome editing agents. Nature 553, 217-221 (2018).
24. Mashiko, D., et al. Generation of mutant mice by pronuclear injection of circular plasmid expressing Cas9 and single guided RNA. Scientific reports 3, 3355 (2013).
25. Zhang, Y., et al. An RNA-sequencing transcriptome and splicing database of glia, neurons, and vascular cells of the cerebral cortex. J Neurosci 34, 11929-11947 (2014).
26. Oakley, H., et al. Intraneuronal beta-amyloid aggregates, neurodegeneration, and neuron loss in transgenic mice with five familial Alzheimer's disease mutations: potential factors in amyloid plaque formation. J Neurosci 26, 10129-10140 (2006).
27. Kimura, R. & Ohno, M. Impairments in remote memory stabilization precede hippocampal synaptic and cognitive failures in 5XFAD Alzheimer mouse model. Neurobiology of disease 33, 229-235 (2009).
28. Shao, C. Y., Mirra, S. S., Sait, H. B., Sacktor, T. C. & Sigurdsson, E. M. Postsynaptic degeneration as revealed by PSD-95 reduction occurs after advanced Abeta and tau pathology in transgenic mouse models of Alzheimer's disease. Acta neuropathologica 122, 285-292 (2011).
29. Gowrishankar, S., et al. Massive accumulation of luminal protease-deficient axonal lysosomes at Alzheimer's disease amyloid plaques. Proceedings of the National Academy of Sciences of the United States of America 112, E3699-3708 (2015).
30. Sharoar, M. G., Hu, X., Ma, X. M., Zhu, X. & Yan, R. Sequential formation of different layers of dystrophic neurites in Alzheimer's brains. Molecular psychiatry 24, 1369-1382 (2019).
31. Spires-Jones, T. L. & Hyman, B. T. The intersection of amyloid beta and tau at synapses in Alzheimer's disease. Neuron 82, 756-771 (2014).
32. Woodhouse, A., West, A. K., Chuckowree, J. A., Vickers, J. C. & Dickson, T. C. Does beta-amyloid plaque formation cause structural injury to neuronal processes? Neurotoxicity research 7, 5-15 (2005).
33. Jankowsky, J. L., et al. Mutant presenilins specifically elevate the levels of the 42 residue beta-amyloid peptide in vivo: evidence for augmentation of a 42-specific gamma secretase. Human molecular genetics 13, 159-170 (2004).
34. Garcia-Alloza, M., et al. Characterization of amyloid deposition in the APPswe/PS1dE9 mouse model of Alzheimer disease. Neurobiology of disease 24, 516-524 (2006).
35. Fu, A. K., et al. IL-33 ameliorates Alzheimer's disease-like pathology and cognitive decline. Proceedings of the National Academy of Sciences of the United States of America 113, E2705-2713 (2016).
36. Lalonde, R., Kim, H. D., Maxwell, J. A. & Fukuchi, K. Exploratory activity and spatial learning in 12-month-old APP (695) SWE/co+PS1/DeltaE9 mice with amyloid plaques. Neuroscience letters 390, 87-92 (2005).
37. Choi, J. H., et al. Optimization of AAV expression cassettes to improve packaging capacity and transgene expression in neurons. Mol Brain 7, 17 (2014).
38. Patricio, M. I., Barnard, A. R., Orlans, H. O., McClements, M. E. & MacLaren, R. E. Inclusion of the Woodchuck Hepatitis Virus Posttranscriptional Regulatory Element Enhances AAV2-Driven Transduction of Mouse and Human Retina. Molecular therapy. Nucleic acids 6, 198-208 (2017).
39. Jawhar, S., Trawicka, A., Jenneckens, C., Bayer, T. A. & Wirths, O. Motor deficits, neuron loss, and reduced anxiety coinciding with axonal degeneration and intraneuronal Abeta aggregation in the 5XFAD mouse model of Alzheimer's disease. Neurobiology of aging 33, 196 e129-140 (2012).
40. Tible, M., et al. PKR knockout in the 5×FAD model of Alzheimer's disease reveals beneficial effects on spatial memory and brain lesions. Aging cell 18, e12887 (2019).
41. Ran, F. A., et al. In vivo genome editing using Staphylococcus aureus Cas9. Nature 520, 186-191 (2015).
42. Cruts, M., Theuns, J. & Van Broeckhoven, C. Locus-specific mutation databases for neurodegenerative brain diseases. Human mutation 33, 1340-1344 (2012).
43. Kleinstiver, B. P., et al. Broadening the targeting range of Staphylococcus aureus CRISPR-Cas9 by modifying PAM recognition. Nature biotechnology 33, 1293-1298 (2015).
44. Nagata, K., et al. Generation of App knock-in mice reveals deletion mutations protective against Alzheimer's disease-like pathology. Nature communications 9, 1800 (2018).
45. Park, H., et al. In vivo neuronal gene editing via CRISPR-Cas9 amphiphilic nanocomplexes alleviates deficits in mouse models of Alzheimer's disease. Nature neuroscience 22, 524-528 (2019).
46. Filser, S., et al. Pharmacological inhibition of BACE1 impairs synaptic plasticity and cognitive functions. Biological psychiatry 77, 729-739 (2015).
47. Ou-Yang, M. H., et al. Axonal organization defects in the hippocampus of adult conditional BACE1 knockout mice. Science translational medicine 10 (2018).
48. Blume, T., et al. BACE1 Inhibitor MK-8931 Alters Formation but Not Stability of Dendritic Spines. Frontiers in aging neuroscience 10, 229 (2018).
49. Ravindra Kumar, S., et al. Multiplexed Cre-dependent selection yields systemic AAVs for targeting distinct brain cell types. Nature methods 17, 541-550 (2020).
50. Flytzanis, N. C., et al. Broad gene expression throughout the mouse and marmoset brain after intravenous delivery of engineered AAV capsids. BioRxiv, 2020.2006.2016.152975 (2020).
51. Bloch, D. B., et al. Sp110 localizes to the PML-Sp100 nuclear body and may function as a nuclear hormone receptor transcriptional coactivator. Mol Cell Biol 20, 6138-6146 (2000).
52. Mo, W. & Zhang, J. T. Human ABCG2: structure, function, and its role in multidrug resistance. Int J Biochem Mol Biol 3, 1-27 (2012).
53. Liang, Z., et al. The pseudokinase CaMKv is required for the activity-dependent maintenance of dendritic spines. Nature communications 7, 13282 (2016).
54. Sun, J., et al. CRISPR/Cas9 editing of APP C-terminus attenuates beta-cleavage and promotes alpha-cleavage. Nature communications 10, 53 (2019).
55. Hocquemiller, M., Giersch, L., Audrain, M., Parker, S. & Cartier, N. Adeno-Associated Virus-Based Gene Therapy for CNS Diseases. Human gene therapy 27, 478-496 (2016).
56. Hudry, E. & Vandenberghe, L. H. Therapeutic AAV Gene Transfer to the Nervous System: A Clinical Reality. Neuron 101, 839-862 (2019).
57. Challis, R. C., et al. Systemic AAV vectors for widespread and targeted gene delivery in rodents. Nature protocols 14, 379-414 (2019).
58. Keane, T. M., et al. Mouse genomic variation and its effect on phenotypes and gene regulation. Nature 477, 289-294 (2011).
59. Wojtowicz, J. M. & Kee, N. BrdU assay for neurogenesis in rodents. Nature protocols 1, 1399-1405 (2006).
60. Styren, S. D., Hamilton, R. L., Styren, G. C. & Klunk, W. E. X-34, a fluorescent derivative of Congo red: a novel histochemical stain for Alzheimer's disease pathology. The journal of histochemistry and cytochemistry: official journal of the Histochemistry Society 48, 1223-1232 (2000).
61. Seo, J., et al. Activity-dependent p25 generation regulates synaptic plasticity and Abeta-induced cognitive impairment. Cell 157, 486-498 (2014).
62. Lee, K., et al. Replenishment of microRNA-188-5p restores the synaptic and cognitive deficits in 5XFAD Mouse Model of Alzheimer's Disease. Scientific reports 6, 34433 (2016).
63. Devi, L. & Ohno, M. Mitochondrial dysfunction and accumulation of the beta-secretase-cleaved C-terminal fragment of APP in Alzheimer's disease transgenic mice. Neurobiology of disease 45, 417-424 (2012).

1. A method for treating or reducing risk of an amyloid pathology in a person in need thereof, comprising the step of administering to the person an effective amount of a composition disrupting amyloid precursor protein (APP) genomic sequence.

2. The method of claim 1, further comprising, prior to the administering step, sequencing at least a portion of the person's genome.

3. The method of claim 1, wherein the person's APP genomic sequence comprises at least one mutation.

4. The method of claim 3, wherein the at least one mutation comprises a Swedish mutation (K670N/M671L), a Florida mutation (I716V), a London mutation (V717I), or any combination thereof.

5. The method of claim 4, wherein the at least one mutation comprises a Swedish mutation (K670N/M671L).

6. The method of claim 1, wherein the amyloid pathology is Alzheimer's Disease (AD), cerebral amyloid angiopathy, or Down Syndrome.

7. The method of claim 6, wherein the AD is familial AD or sporadic AD.

8. The method of claim 1, wherein the person has been diagnosed with AD, or the person is not yet diagnosed with AD but has known risk factors for AD.

9. The method of claim 1, wherein the composition comprising an siRNA, a microRNA, a miniRNA, a lncRNA, or an antisense oligonucleotide targeting the APP genomic sequence.

10. The method of claim 1, wherein the composition comprising one or more vectors encoding (1) an endonuclease guided by a small guide RNA (sgRNA) targeting a location within the APP genomic sequence; and (2) the sgRNA.

11. The method of claim 1, wherein the sgRNA targets a region within the APP genomic sequence comprising the at least one mutation or a β- or γ-secretase cleavage site.

12. The method of claim 10, wherein the composition comprises one vector encoding a Cas9 nuclease and one sgRNA, preferably a Streptococcus pyogenes Cas9 nuclease (SpCas9).

13. The method of claim 10, wherein the one or more vectors are one viral vector, preferably an adeno-associated virus (AAV) vector.

14. The method of claim 1, wherein the composition is administered by subcutaneous, intramuscular, intravenous, intraperitoneal, or intracranial injection or by oral or nasal administration.

15. The method of claim 13, wherein the composition is administered in the form of a solution, a suspension, a powder, a paste, a tablet, or a capsule.

16. A kit for treating or reducing risk of an amyloid pathology in a person in need thereof, comprising a container containing a composition disrupting APP genomic sequence.

17. The kit of claim 16, wherein the composition is formulated for subcutaneous, intramuscular, intravenous, intraperitoneal, or intracranial injection, or for oral or nasal administration.

18. The kit of claim 16, wherein the composition comprising an siRNA, a microRNA, a miniRNA, a lncRNA, or an antisense oligonucleotide targeting the APP genomic sequence.

19. The kit of claim 16, wherein the composition comprising one or more vectors encoding (1) an endonuclease guided by a small guide RNA (sgRNA) targeting one location within the APP genomic sequence; and (2) the sgRNA.

20. The kit of claim 19, wherein the sgRNA targets a region within the APP genomic sequence comprising the at least one mutation or a β- or γ-secretase cleavage site.

21. The kit of claim 16, wherein the composition comprises one vector encoding a Cas9 nuclease and one sgRNA, preferably a Streptococcus pyogenes Cas9 nuclease (SpCas9).

22. The kit of claim 19, wherein the one or more vectors are one or more viral vectors, preferably an adeno-associated virus (AAV) vector.

23. The kit of claim 16, wherein the composition is administered by subcutaneous, intramuscular, intravenous, intraperitoneal, or intracranial injection or by oral or nasal administration.

24. The kit of claim 16, further comprising a second container containing agents for sequencing at least a portion of the person's genome.

25. The kit of claim 16, further comprising an instruction manual for administration of the composition.


SEQUENCE LISTING (89 sequences)

SEQ ID NO: 1 - DNA, 4889 bp, synthetic construct
  misc_feature 1..4889: note = Description of sequence: nucleotide sequence of the sgRNA-Syn::Cas9-mWPRE construct ITR-U6-SW1-Syn-SaCas9-HA-mWPRE-pA-ITR
  source 1..4889: mol_type = other DNA
cctgcaggcagctgcgcgctcgctcgctcactgaggccgcccgggcgtcgggcgacctttggtcgcccggcctcagtgagcgagcgagcgcgcagagagggagtggccaactccatcactaggggttcctgcggcctctagagagggcctatttcccatgattccttcatatttgcatatacgatacaaggctgttagagagataattggaattaatttgactgtaaacacaaagatattagtacaaaatacgtgacgtagaaagtaataatttcttgggtagtttgcagttttaaaattatgttttaaaatggactatcatatgcttaccgtaacttgaaagtatttcgatttcttggctttatatatcttgtggaaaggacgaaacaccgtctctgaagtgaatctggatggttttagtactctggaaacagaatctactaaaacaaggcaaaatgccgtgtttatctcgtcaacttgttggcgagattttttctagaagtgcaagtgggttttaggaccaggatgaggcggggtgggggtgcctacctgacgaccgaccccgacccactggacaagcacccaacccccattccccaaattgcgcatcccctatcagagagggggaggggaaacaggatgcggcgaggcgcgtgcgcactgccagcttcagcaccgcggacagtgccttcgcccccgcctggcggcgcgcgccaccgccgcctcagcactgaaggcgcgctgacgtcactcgccggtcccccgcaaactccccttcccggccaccttggtcgcgtccgcgccgccgccggcccagccggaccgcaccacgcgaggcgcgagataggggggcacgggcgcgaccatctgcgctgcggcgccggcgactcagcgctgcctcagtctgcggtgggcagcggaggagtcgtgtcgtgcctgagagcgcaggccaccatggccccaaagaagaagcggaaggtcggtatccacggagtcccagcagccaagcggaactacatcctgggcctggacatcggcatcaccagcgtgggctacggcatcatcgactacgagacacgggacgtgatcgatgccggcgtgcggctgttcaaagaggccaacgtggaaaacaacgagggcaggcggagcaagagaggcgccagaaggctgaagcggcggaggcggcatagaatccagagagtgaagaagctgctgttcgactacaacctgctgaccgaccacagcgagctgagcggcatcaacccctacgaggccagagtgaagggcctgagccagaagctgagcgaggaagagttctctgccgccctgctgcacctggccaagagaagaggcgtgcacaacgtgaacgaggtggaagaggacaccggcaacgagctgtccaccaaagagcagatcagccggaacagcaaggccctggaagagaaatacgtggccgaactgcagctggaacggctgaagaaagacggcgaagtgcggggcagcatcaacagattcaagaccagcgactacgtgaaagaagccaaacagctgctgaaggtgcagaaggcctaccaccagctggaccagagcttcatcgacacctacatcgacctgctggaaacccggcggacctactatgagggacctggcgagggcagccccttcggctggaaggacatcaaagaatggtacgagatgctgatgggccactgcacctacttccccgaggaactgcggagcgtgaagtacgcctacaacgccgacctgtacaacgccctgaacgacctgaacaatctcgtgatcaccagggacgagaacgagaagctggaatattacgagaagttccagatcatcgagaacgtgttcaagcagaagaagaagcccaccctgaagcagatcgccaaagaaatcctcgtgaacgaagaggatattaagggctacagagtgaccagcaccggcaagcccgagttcaccaacctgaaggtgtaccacgacatcaaggacattaccgcccggaaagagattattgagaacgccgagctgctggatcagattgccaagatcctgaccatctaccagagcagcgaggacatccaggaagaactgaccaatctgaactccgagctgacccaggaagagatcgagcagatctctaatctgaagggctataccggcacccacaacctgagcctgaaggccatcaacctgatcctggacgagctgtggcacaccaacgacaaccagatcgctatcttcaaccggctgaagctggtgcccaagaaggtggacctgtcccagcagaaagagatccccaccaccctggtggacgacttcatcctgagccccgtcgtgaagagaagcttcatccagagcatcaaagtgatcaacgccatcatcaagaagtacggcctgcccaacgacatcattatcgagctggcccgcgagaagaactccaaggacgcccagaaaatgatcaacgagatgcagaagcggaaccggcagaccaacgagcggatcgaggaaatcatccggaccaccggcaaagagaacgccaagtacctgatcgagaagatcaagctgcacgacatgcaggaaggcaagtgcctgtacagcctggaagccatccctctggaagatctgctgaacaaccccttcaactatgaggtggaccacatcatccccagaagcgtgtccttcgacaacagcttcaacaacaaggtgctcgtgaagcaggaagaaaacagcaagaagggcaaccggaccccattccagtacctgagcagcagcgacagcaagatcagctacgaaaccttcaagaagcacatcctgaatctggccaagggcaagggcagaatcagcaagaccaagaaagagtatctgctggaagaacgggacatcaacaggttctccgtgcagaaagacttcatcaaccggaacctggtggataccagatacgccaccagaggcctgatgaacctgctgcggagctacttcagagtgaacaacctggacgtgaaagtgaagtccatcaatggcggcttcaccagctttctgcggcggaagtggaagtttaagaaagagcggaacaaggggtacaagcaccacgccgaggacgccctgatcattgccaacgccgatttcatcttcaaagagtggaagaaactggacaaggccaaaaaagtgatggaaaaccagatgttcgaggaaaagcaggccgagagcatgcccgagatcgaaaccgagcaggagtacaaagagatcttcatcaccccccaccagatcaagcacattaaggacttcaaggactacaagtacagccaccgggtggacaagaagcctaatagagagctgattaacgacaccctgtactccacccggaaggacgacaagggcaacaccctgatcgtgaacaatctgaacggcctgtacgacaaggacaatgacaagctgaaaaagctgatcaacaagagccccgaaaagctgctgatgtaccaccacgacccccagacctaccagaaactgaagctgattatggaacagtacggcgacgagaagaatcccctgtacaagtactacgaggaaaccgggaactacctgaccaagtactccaaaaaggacaacggccccgtgatcaagaagattaagtattacggcaacaaactgaacgcccatctggacatcaccgacgactaccccaacagcagaaacaaggtcgtgaagctgtccctgaagccctacagattcgacgtgtacctggacaatggcgtgtacaagttcgtgaccgtgaagaatctggatgtgatcaaaaaagaaaactactacgaagtgaatagcaagtgctatgaggaagctaagaagctgaagaagatcagcaaccaggccgagtttatcgcctccttctacaacaacgatctgatcaagatcaacggcgagctgtatagagtgatcggcgtgaacaacgacctgctgaaccggatcgaagtgaacatgatcgacatcacctaccgcgagtacctggaaaacatgaacgacaagaggccccccaggatcattaagacaatcgcctccaagacccagagcattaagaagtacagcacagacattctgggcaacctgtatgaagtgaaatctaagaagcaccctcagatcatcaaaaagggcaaaaggccggcggccacgaaaaaggccggccaggcaaaaaagaaaaagggatcctacccatacgatgttccagattacgcttacccatacgatgttccagattacgcttacccatacgatgttccagattacgcttaagaattccgctcgagataatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttagttcttgccacggcggaactcatcgccgcctgccttgcccgctgctggacaggggctcggctgttgggcactgacaattccgtggtgtttatttgtgaaatttgtgatgctattgctttatttgtaaccatctagctttatttgtgaaatttgtgatgctattgctttatttgtaaccattataagctgcaataaacaagttaacaacaacaattgcattcattttatgtttcaggttcagggggagatgtgggaggttttttaaagcggccgcaggaacccctagtgatggagttggccactccctctctgcgcgctcgctcgctcactgaggccgggcgaccaaaggtcgcccgacgcccgggctttgcccgggcggcctcagtgagcgagcgagcgcgcagctgcctgcagg

SEQ ID NO: 2 - protein, 695 aa, synthetic construct
  REGION 1..695: note = Description of sequence: APP amino acid sequence
  DOMAIN 612..613: note = alpha-secretase site
  DOMAIN 596..597: note = beta-secretase site
  DOMAIN 636..638: note = gamma-secretase site
  DOMAIN 645..646: note = gamma-secretase site
MLPGLALLLLAAWTARALEVPTDGNAGLLAEPQIAMFCGRLNMHMNVQNGKWDSDPSGTKTCIDTKEGILQYCQEVYPELQITNVVEANQPVTIQNWCKRGRKQCKTHPHFVIPYRCLVGEFVSDALLVPDKCKFLHQERMDVCETHLHWHTVAKETCSEKSTNLHDYGMLLPCGIDKFRGVEFVCCPLAEESDNVDSADAEEDDSDVWWGGADTDYADGSEDKVVEVAEEEEVAEVEEEEADDDEDDEDGDEVEEEAEEPYEEATERTTSIATTTTTTTESVEEVVRVPTTAASTPDAVDKYLETPGDENEHAHFQKAKERLEAKHRERMSQVMREWEEAERQAKNLPKADKKAVIQHFQEKVESLEQEAANERQQLVETHMARVEAMLNDRRRLALENYITALQAVPPRPRHVFNMLKKYVRAEQKDRQHTLKHFEHVRMVDPKKAAQIRSQVMTHLRVIYERMNQSLSLLYNVPAVAEEIQDEVDELLQKEQNYSDDVLANMISEPRISYGNDALMPSLTETKTTVELLPVNGEFSLDDLQPWHSFGADSVPANTENEVEPVDARPAADRGLTTRPGSGLTNIKTEEISEVKMDAEFRHDSGYEVHHQKLVFFAEDVGSNKGAIIGLMVGGVVIATVIVITLVMLKKKQYTSIHHGVVEVDAAVTPEERHLSKMQQNGYENPTYKFFEQMQN

SEQ ID NO: 3 - DNA, 222 bp, synthetic construct
  misc_feature 1..222: note = Description of sequence: Exon 14 in APP
  source 1..222: mol_type = other DNA
atgagctgcttcagaaagagcaaaactattcagatgacgtcttggccaacatgattagtgaaccaaggatcagttacggaaacgatgctctcatgccatctttgaccgaaacgaaaaccaccgtggagctccttcccgtgaatggagagttcagcctggacgatctccagccgtggcattcttttggggctgactctgtgccagccaacacagaaaacgaag

SEQ ID NO: 4 - DNA, 54 bp, synthetic construct
  misc_feature 1..54: note = Description of sequence: Exon 15 in APP
  source 1..54: mol_type = other DNA
ttgagcctgttgatgcccgccctgctgccgaccgaggactgaccactcgaccag

SEQ ID NO: 5 - DNA, 101 bp, synthetic construct
  misc_feature 1..101: note = Description of sequence: Exon 16 in APP
  source 1..101: mol_type = other DNA
gttctgggttgacaaatatcaagacggaggagatctctgaagtgaagatggatgcagaattccgacatgactcaggatatgaagttcatcatcaaaaattg

SEQ ID NO: 6 - DNA, 147 bp, synthetic construct
  misc_feature 1..147: note = Description of sequence: Exon 17 in APP
  source 1..147: mol_type = other DNA
gtgttctttgcagaagatgtgggttcaaacaaaggtgcaatcattggactcatggtgggcggtgttgtcatagcgacagtgatcgtcatcaccttggtgatgctgaagaagaaacagtacacatccattcatcatggtgtggtggag

SEQ ID NO: 7 - DNA, 27 bp, synthetic construct
  misc_feature 1..27: note = Description of sequence: Cas9 or variant type: SaCas9, Name: APP1
  source 1..27: mol_type = other DNA
  misc_feature 22..27: note = PAM
tcatgttggccaagacgtcatctgaat

SEQ ID NO: 8 - DNA, 27 bp, synthetic construct
  misc_feature 1..27: note = Description of sequence: Cas9 or variant type: SaCas9, Name: APP2
  source 1..27: mol_type = other DNA
  misc_feature 22..27: note = PAM
ggccaacatgattagtgaaccaaggat

SEQ ID NO: 9 - DNA, 27 bp, synthetic construct
  misc_feature 1..27: note = Description of sequence: Cas9 or variant type: SaCas9, Name: APP3
  source 1..27: mol_type = other DNA
  misc_feature 22..27: note = PAM
accaccgtggagctccttcccgtgaat

SEQ ID NO: 10 - DNA, 27 bp, synthetic construct
  misc_feature 1..27: note = Description of sequence: Cas9 or variant type: SaCas9, Name: APP4
  source 1..27: mol_type = other DNA
  misc_feature 22..27: note = PAM
tggagctccttcccgtgaatggagagt

SEQ ID NO: 11 - DNA, 27 bp, synthetic construct
  misc_feature 1..27: note = Description of sequence: Cas9 or variant type: SaCas9, Name: APP5
  source 1..27: mol_type = other DNA
  misc_feature 22..27: note = PAM
ctggcacagagtcagccccaaaagaat

SEQ ID NO: 12 - DNA, 27 bp, synthetic construct
  misc_feature 1..27: note = Description of sequence: Cas9 or variant type: SaCas9, Name: APP6
  source 1..27: mol_type = other DNA
  misc_feature 22..27: note = PAM
cgttttctgtgttggctggcacagagt

SEQ ID NO: 13 - DNA, 27 bp, synthetic construct
  misc_feature 1..27: note = Description of sequence: Cas9 or variant type: SaCas9, Name: APP7
  source 1..27: mol_type = other DNA
  misc_feature 22..27: note = PAM
gaggagatctctgaagtgaagatggat

SEQ ID NO: 14 - DNA, 27 bp, synthetic construct
  misc_feature 1..27: note = Description of sequence: Cas9 or variant type: SaCas9, Name: APP8
  source 1..27: mol_type = other DNA
  misc_feature 22..27: note = PAM
tctctgaagtgaagatggatgcagaat

SEQ ID NO: 15 - DNA, 27 bp, synthetic construct
  misc_feature 1..27: note = Description of sequence: Cas9 or variant type: SaCas9, Name: APP9
  source 1..27: mol_type = other DNA
  misc_feature 22..27: note = PAM
acttcatatcctgagtcatgtcggaat

SEQ ID NO: 16 - DNA, 27 bp, synthetic construct
  misc_feature 1..27: note = Description of sequence: Cas9 or variant type: SaCas9, Name: APP10
  source 1..27: mol_type = other DNA
  misc_feature 22..27: note = PAM
tttgatgatgaacttcatatcctgagt

SEQ ID NO: 17 - DNA, 27 bp, synthetic construct
  misc_feature 1..27: note = Description of sequence: Cas9 or variant type: SaCas9, Name: APP11
  source 1..27: mol_type = other DNA
  misc_feature 22..27: note = PAM
atgcagaattccgacatgactcaggat

SEQ ID NO: 18 - DNA, 27 bp, synthetic construct
  misc_feature 1..27: note = Description of sequence: Cas9 or variant type: SaCas9, Name: APP12
  source 1..27: mol_type = other DNA
  misc_feature 22..27: note = PAM
gctatgacaacaccgcccaccatgagt

SEQ ID NO: 19 - DNA, 27 bp, synthetic construct
  misc_feature 1..27: note = Description of sequence: Cas9 or variant type: SaCas9KKH, Name: APP13
  source 1..27: mol_type = other DNA
  misc_feature 22..27: note = PAM
cagatgacgtcttggccaacaaatagt

SEQ ID NO: 20 - DNA, 27 bp, synthetic construct
  misc_feature 1..27: note = Description of sequence: Cas9 or variant type: SaCas9KKH, Name: APP14
  source 1..27: mol_type = other DNA
  misc_feature 22..27: note = PAM
gacgtcttggccaacatgatttcagat

SEQ ID NO: 21 - DNA, 27 bp, synthetic construct
  misc_feature 1..27: note = Description of sequence: Cas9 or variant type: SaCas9KKH, Name: APP15
  source 1..27: mol_type = other DNA
  misc_feature 22..27: note = PAM
ttcagatgacgtcttggccaacatgat

SEQ ID NO: 22 - DNA, 27 bp, synthetic construct
  misc_feature 1..27: note = Description of sequence: Cas9 or variant type: SaCas9KKH, Name: APP16
  source 1..27: mol_type = other DNA
  misc_feature 22..27: note = PAM
gatgacgtcttggccaacatgattagt

SEQ ID NO: 23 - DNA, 27 bp, synthetic construct
  misc_feature 1..27: note = Description of sequence: Cas9 or variant type: SaCas9KKH, Name: APP17
  source 1..27: mol_type = other DNA
  misc_feature 22..27: note = PAM
gcatcgtttccgtaactgatccttggt

SEQ ID NO: 24 - DNA, 27 bp, synthetic construct
  misc_feature 1..27: note = Description of sequence: Cas9 or variant type: SaCas9KKH, Name: APP18
  source 1..27: mol_type = other DNA
  misc_feature 22..27: note = PAM
aacatgattagtgaaccaaggatcagt

SEQ ID NO: 25 - DNA, 27 bp, synthetic construct
  misc_feature 1..27: note = Description of sequence: Cas9 or variant type: SaCas9KKH, Name: APP19
  source 1..27: mol_type = other DNA
  misc_feature 22..27: note = PAM
gaaccaaggatcagttacggaaacgat

SEQ ID NO: 26 - DNA, 27 bp, synthetic construct
  misc_feature 1..27: note = Description of sequence: Cas9 or variant type: SaCas9KKH, Name: APP20
  source 1..27: mol_type = other DNA
  misc_feature 22..27: note = PAM
acggtggttttcgtttcggtcaaagat

SEQ ID NO: 27 - DNA, 27 bp, synthetic construct
  misc_feature 1..27: note = Description of sequence: Cas9 or variant type: SaCas9KKH, Name: APP21
  source 1..27: mol_type = other DNA
  misc_feature 22..27: note = PAM
gagctccacggtggttttcgtttcggt

SEQ ID NO: 28 - DNA, 27 bp, synthetic construct
  misc_feature 1..27: note = Description of sequence: Cas9 or variant type: SaCas9KKH, Name: APP22
  source 1..27: mol_type = other DNA
  misc_feature 22..27: note = PAM
attcacgggaaggagctccacggtggt

SEQ ID NO: 29 - DNA, 27 bp, synthetic construct
  misc_feature 1..27: note = Description of sequence: Cas9 or variant type: SaCas9KKH, Name: APP23
  source 1..27: mol_type = other DNA
  misc_feature 22..27: note = PAM
tccattcacgggaaggagctccacggt

SEQ ID NO: 30 - DNA, 27 bp, synthetic construct
  misc_feature 1..27: note = Description of sequence: Cas9 or variant type: SaCas9KKH, Name: APP24
  source 1..27: mol_type = other DNA
  misc_feature 22..27: note = PAM
gtgaatggagagttcagcctggacgat

SEQ ID NO: 31 - DNA, 27 bp, synthetic construct
  misc_feature 1..27: note = Description of sequence: Cas9 or variant type: SaCas9KKH, Name: APP25
  source 1..27: mol_type = other DNA
  misc_feature 22..27: note = PAM
agccgtggcattcttttggggggagat

SEQ ID NO: 32 - DNA, 27 bp, synthetic construct
  misc_feature 1..27: note = Description of sequence: Cas9 or variant type: SaCas9KKH, Name: APP26
  source 1..27: mol_type = other DNA
  misc_feature 22..27: note = PAM
acggaggagatctctgaagtgcttgat

SEQ ID NO: 33 - DNA, 27 bp, synthetic construct
  misc_feature 1..27: note = Description of sequence: Cas9 or variant type: SaCas9KKH, Name: APP27
  source 1..27: mol_type = other DNA
  misc_feature 22..27: note = PAM
gttgacaaatatcaagacggaggagat

SEQ ID NO: 34 - DNA, 27 bp, synthetic construct
  misc_feature 1..27: note = Description of sequence: Cas9 or variant type: SaCas9KKH, Name: APP28
  source 1..27: mol_type = other DNA
  misc_feature 22..27: note = PAM
gaagtgaagatggatgcagaaagagat

SEQ ID NO: 35 - DNA, 27 bp, synthetic construct
  misc_feature 1..27: note = Description of sequence: Cas9 or variant type: SaCas9KKH, Name: APP29
  source 1..27: mol_type = other DNA
  misc_feature 22..27: note = PAM
tatcaagacggaggagatctctgaagt

SEQ ID NO: 36 - DNA, 27 bp, synthetic construct
  misc_feature 1..27: note = Description of sequence: Cas9 or variant type: SaCas9KKH, Name: APP30
  source 1..27: mol_type = other DNA
  misc_feature 22..27: note = PAM
gacggaggagatctctgaagtgaagat

SEQ ID NO: 37 - DNA, 27 bp, synthetic construct
  misc_feature 1..27: note = Description of sequence: Cas9 or variant type: SaCas9KKH, Name: APP31
  source 1..27: mol_type = other DNA
  misc_feature 22..27: note = PAM
attccgacatgactcaggatatgaagt

SEQ ID NO: 38 - DNA, 27 bp, synthetic construct
  misc_feature 1..27: note = Description of sequence: Cas9 or variant type: SaCas9KKH, Name: APP32
  source 1..27: mol_type = other DNA
  misc_feature 22..27: note = PAM
caggatatgaagttcatcatcaaaaat

SEQ ID NO: 39 - DNA, 27 bp, synthetic construct
  misc_feature 1..27: note = Description of sequence: Cas9 or variant type: SaCas9KKH, Name: APP33
  source 1..27: mol_type = other DNA
  misc_feature 22..27: note = PAM
gcagaagatgtgggttcaaacaaaggt

SEQ ID NO: 40 - DNA, 27 bp, synthetic construct
  misc_feature 1..27: note = Description of sequence: Cas9 or variant type: SaCas9KKH, Name: APP34
  source 1..27: mol_type = other DNA
  misc_feature 22..27: note = PAM
agatgtgggttcaaacaaaggtgcaat

SEQ ID NO: 41 - DNA, 27 bp, synthetic construct
  misc_feature 1..27: note = Description of sequence: Cas9 or variant type: SaCas9KKH, Name: APP35
  source 1..27: mol_type = other DNA
  misc_feature 22..27: note = PAM
aacaccgcccaccatgagtccaatgat

SEQ ID NO: 42 - DNA, 27 bp, synthetic construct
  misc_feature 1..27: note = Description of sequence: Cas9 or variant type: SaCas9KKH, Name: APP36
  source 1..27: mol_type = other DNA
  misc_feature 22..27: note = PAM
gacaacaccgcccaccatgagtccaat

SEQ ID NO: 43 - DNA, 27 bp, synthetic construct
  misc_feature 1..27: note = Description of sequence: Cas9 or variant type: SaCas9KKH, Name: APP37
  source 1..27: mol_type = other DNA
  misc_feature 22..27: note = PAM
caaaggtgcaatcattggactcatggt

SEQ ID NO: 44 - DNA, 27 bp, synthetic construct
  misc_feature 1..27: note = Description of sequence: Cas9 or variant type: SaCas9KKH, Name: APP38
  source 1..27: mol_type = other DNA
  misc_feature 22..27: note = PAM
gcaatcattggactcatggtgggcggt

SEQ ID NO: 45 - DNA, 27 bp, synthetic construct
  misc_feature 1..27: note = Description of sequence: Cas9 or variant type: SaCas9KKH, Name: APP39
  source 1..27: mol_type = other DNA
  misc_feature 22..27: note = PAM
ggtgggcggtgttgtcatagcgacagt

SEQ ID NO: 46 - DNA, 27 bp, synthetic construct
  misc_feature 1..27: note = Description of sequence: Cas9 or variant type: SaCas9KKH, Name: APP40
  source 1..27: mol_type = other DNA
  misc_feature 22..27: note = PAM
gggcggtgttgtcatagcgacagtgat

SEQ ID NO: 47 - DNA, 27 bp, synthetic construct
  misc_feature 1..27: note = Description of sequence: Cas9 or variant type: SaCas9KKH, Name: APP41
  source 1..27: mol_type = other DNA
  misc_feature 22..27: note = PAM
cttcagcatcaccaaggtgatgacgat

SEQ ID NO: 48 - DNA, 27 bp, synthetic construct
  misc_feature 1..27: note = Description of sequence: Cas9 or variant type: SaCas9KKH, Name: APP42
  source 1..27: mol_type = other DNA
  misc_feature 22..27: note = PAM
tttcttcttcagcatcaccaaggtgat

SEQ ID NO: 49 - DNA, 27 bp, synthetic construct
  misc_feature 1..27: note = Description of sequence: Cas9 or variant type: SaCas9KKH, Name: APP43
  source 1..27: mol_type = other DNA
  misc_feature 22..27: note = PAM
ctgtttcttcttcagcatcaccaaggt

SEQ ID NO: 50 - DNA, 27 bp, synthetic construct
  misc_feature 1..27: note = Description of sequence: Cas9 or variant type: SaCas9KKH, Name: APP44
  source 1..27: mol_type = other DNA
  misc_feature 22..27: note = PAM
agcgacagtgatcgtcatcaccttggt

SEQ ID NO: 51 - DNA, 27 bp, synthetic construct
  misc_feature 1..27: note = Description of sequence: Cas9 or variant type: SaCas9KKH, Name: APP45
  source 1..27: mol_type = other DNA
  misc_feature 22..27: note = PAM
gacagtgatcgtcatcaccttggtgat

SEQ ID NO: 52 - DNA, 27 bp, synthetic construct
  misc_feature 1..27: note = Description of sequence: Cas9 or variant type: SaCas9KKH, Name: APP46
  source 1..27: mol_type = other DNA
  misc_feature 22..27: note = PAM
ccttggtgatgctgaagaagaaacagt

SEQ ID NO: 53 - DNA, 27 bp, synthetic construct
  misc_feature 1..27: note = Description of sequence: Cas9 or variant type: SaCas9KKH, Name: APP47
  source 1..27: mol_type = other DNA
  misc_feature 22..27: note = PAM
aaacagtacacatccattcatcatggt

SEQ ID NO: 54 - DNA, 27 bp, synthetic construct
  misc_feature 1..27: note = Description of sequence: Cas9 or variant type: SaCas9KKH, Name: APP48
  source 1..27: mol_type = other DNA
  misc_feature 22..27: note = PAM
gtacacatccattcatcatggtgtggt

SEQ ID NO: 55 - DNA, 29 bp, synthetic construct
  misc_feature 1..29: note = Description of sequence: Cas9 or variant type: CjCas9, Name: APP49
  source 1..29: mol_type = other DNA
  misc_feature 22..29: note = PAM
tttggggctgactctgtgccagccaacac

SEQ ID NO: 56 - DNA, 29 bp, synthetic construct
  misc_feature 1..29: note = Description of sequence: Cas9 or variant type: CjCas9, Name: APP50
  source 1..29: mol_type = other DNA
  misc_feature 22..29: note = PAM
tttgaacccacatcttctgcaaagaacac

SEQ ID NO: 57 - DNA, 29 bp, synthetic construct
  misc_feature 1..29: note = Description of sequence: Cas9 or variant type: CjCas9, Name: APP51
  source 1..29: mol_type = other DNA
  misc_feature 22..29: note = PAM
accgcccaccatgagtccaatgattgcac

SEQ ID NO: 58 - DNA, 29 bp, synthetic construct
  misc_feature 1..29: note = Description of sequence: Cas9 or variant type: CjCas9, Name: APP52
  source 1..29: mol_type = other DNA
  misc_feature 22..29: note = PAM
gatgacgatcactgtcgctatgacaacac

SEQ ID NO: 59 - DNA, 29 bp, synthetic construct
  misc_feature 1..29: note = Description of sequence: Cas9 or variant type: CjCas9, Name: APP53
  source 1..29: mol_type = other DNA
  misc_feature 22..29: note = PAM
ccttggtgatgctgaagaagaaacagtac

SEQ ID NO: 60 - DNA, 29 bp, synthetic construct
  misc_feature 1..29: note = Description of sequence: Cas9 or variant type: CjCas9, Name: APP54
  source 1..29: mol_type = other DNA
  misc_feature 22..29: note = PAM
ttggtgatgctgaagaagaaacagtacac

SEQ ID NO: 61 - DNA, 29 bp, synthetic construct
  misc_feature 1..29: note = Description of sequence: Cas9 or variant type: CjCas9, Name: APP55
  source 1..29: mol_type = other DNA
  misc_feature 22..29: note = PAM
ccaccacaccatgatgaatggatgtgtac

SEQ ID NO: 62 - DNA, 27 bp, synthetic construct
  misc_feature 1..27: note = Description of sequence: sgRNA, name: SW1
  source 1..27: mol_type = other DNA
  misc_feature 22..27: note = PAM
tctctgaagtgaatctggatgcagaat

SEQ ID NO: 63 - DNA, 27 bp, synthetic construct
  misc_feature 1..27: note = Description of sequence: sgRNA, name: SW2
  source 1..27: mol_type = other DNA
  misc_feature 22..27: note = PAM
gaggagatctctgaagtgaatctggat

SEQ ID NO: 64 - DNA, 27 bp, synthetic construct
  misc_feature 1..27: note = Description of sequence: sgRNA, name: WT1
  source 1..27: mol_type = other DNA
  misc_feature 22..27: note = PAM
tctctgaagtgaagatggatgcagaat

SEQ ID NO: 65 - DNA, 27 bp, synthetic construct
  misc_feature 1..27: note = Description of sequence: sgRNA, name: WT2
  source 1..27: mol_type = other DNA
  misc_feature 22..27: note = PAM
gaggagatctctgaagtgaagatggat

SEQ ID NO: 66 - DNA, 20 bp, synthetic construct
  misc_feature 1..20: note = Description of sequence: sgRNA, name: CON
  source 1..20: mol_type = other DNA
ggagaccacggcaggtctca

SEQ ID NO: 67 - protein, 18 aa, synthetic construct
  REGION 1..18: note = Description of sequence: APP Swedish (APPswe) double-base mutation peptide
IKTEEISEVNLDAEFRHD

SEQ ID NO: 68 - DNA, 54 bp, synthetic construct
  misc_feature 1..54: note = Description of sequence: APP Swedish (APPswe) double-base mutation sequence
  source 1..54: mol_type = other DNA
atcaagacggaggagatctctgaagtgaatctggatgcagaattccgacatgac

SEQ ID NO: 69 - DNA, 27 bp, synthetic construct
  misc_feature 1..27: note = Description of sequence: SW1
  source 1..27: mol_type = other DNA
tctctgaagtgaatctggatgcagaat

SEQ ID NO: 70 - DNA, 27 bp, synthetic construct
  misc_feature 1..27: note = Description of sequence: Sw2
  source 1..27: mol_type = other DNA
gaggagatctctgaagtgaatctggat

SEQ ID NO: 71 - DNA, 22 bp, synthetic construct
  misc_feature 1..22: note = Description of sequence: Sequence from FIG. 8, Name: sw1
  source 1..22: mol_type = other DNA
tctctgaagtgaatctggatgc

SEQ ID NO: 72 - DNA, 21 bp, synthetic construct
  misc_feature 1..21: note = Description of sequence: Sequence from FIG. 8, Name: oFF1
  source 1..21: mol_type = other DNA
tttctgaagtgaatctggaag

SEQ ID NO: 73 - DNA, 21 bp, synthetic construct
  misc_feature 1..21: note = Description of sequence: Sequence from FIG. 8, Name: Off2
  source 1..21: mol_type = other DNA
aatctaaattaaatctggatg

SEQ ID NO: 74 - DNA, 21 bp, synthetic construct
  misc_feature 1..21: note = Description of sequence: Sequence from FIG. 8, Name: Off3
  source 1..21: mol_type = other DNA
tcacataaatgaatctggatc

SEQ ID NO: 75 - DNA, 21 bp, synthetic construct
  misc_feature 1..21: note = Description of sequence: Sequence from FIG. 8, Name: Off4
  source 1..21: mol_type = other DNA
attctgctgtgaatctggatg

SEQ ID NO: 76 - DNA, 21 bp, synthetic construct
  misc_feature 1..21: note = Description of sequence: Sequence from FIG. 8, Name: Off5
  source 1..21: mol_type = other DNA
gatctgaggtgactctggatg

SEQ ID NO: 77 - DNA, 35 bp, synthetic construct
  misc_feature 1..35: note = Description of sequence: Sequence from FIG. 8, Top 10 most abundant APP-swe sequence
  source 1..35: mol_type = other DNA
gagatctctgaagtgaatctggatgcagaattgga

SEQ ID NO: 78 - DNA, 35 bp, synthetic construct; identical to SEQ ID NO: 77
  misc_feature 1..35: note = Description of sequence: Sequence from FIG. 8, Top 10 most abundant APP-swe sequence
  source 1..35: mol_type = other DNA

SEQ ID NO: 79 - DNA, 35 bp, synthetic construct
  misc_feature 1..35: note = Description of sequence: Sequence from FIG. 8, Top 10 most abundant APP-swe sequence
  source 1..35: mol_type = other DNA
gagatttctgaagtgaatctggatgcagaattgga

SEQ ID NO: 80 - DNA, 34 bp, synthetic construct
  misc_feature 1..34: note = Description of sequence: Sequence from FIG. 8, Top 10 most abundant APP-swe sequence
  source 1..34: mol_type = other DNA
gagatctctgaagtgaatctggtgcagaattgga

SEQ ID NO: 81 - DNA, 31 bp, synthetic construct
  misc_feature 1..31: note = Description of sequence: Sequence from FIG. 8, Top 10 most abundant APP-swe sequence
  source 1..31: mol_type = other DNA
gagatctctgaagtgaatctggagaattgga

SEQ ID NO: 82 - DNA, 31 bp, synthetic construct
  misc_feature 1..31: note = Description of sequence: Sequence from FIG. 8, Top 10 most abundant APP-swe sequence
  source 1..31: mol_type = other DNA
gagatctctgaagtgaatatgcagaattgga

SEQ ID NO: 83 - DNA, 36 bp, synthetic construct
  misc_feature 1..36: note = Description of sequence: Sequence from FIG. 8, Top 10 most abundant APP-swe sequence
  source 1..36: mol_type = other DNA
gagatctctgaagtgaatctggtattgcagaattcg

SEQ ID NO: 84 - DNA, 34 bp, synthetic construct
  misc_feature 1..34: note = Description of sequence: Sequence from FIG. 8, Top 10 most abundant APP-swe sequence
  source 1..34: mol_type = other DNA
gagatctctgaagtgaatctgatgcagaattgga

SEQ ID NO: 85 - DNA, 33 bp, synthetic construct
  misc_feature 1..33: note = Description of sequence: Sequence from FIG. 8, Top 10 most abundant APP-swe sequence
  source 1..33: mol_type = other DNA
gagatctctgaagtgaatctgggcagaattgga

SEQ ID NO: 86 - DNA, 32 bp, synthetic construct
  misc_feature 1..32: note = Description of sequence: Sequence from FIG. 8, Top 10 most abundant APP-swe sequence
  source 1..32: mol_type = other DNA
gagatctctgaagtgaatctggcagaattgga

SEQ ID NO: 87 - DNA, 35 bp, synthetic construct
  misc_feature 1..35: note = Description of sequence: Sequence from FIG. 8, Top 10 most abundant APP-swe sequence
  source 1..35: mol_type = other DNA
gagatctctaaagtgaatctggatgcagaattgga

SEQ ID NO: 88 - protein, 770 aa, synthetic construct
  REGION 1..770: note = Description of sequence: Cas9-sgRNA complex
MLPGLALLLLAAWTARALEVPTDGNAGLLAEPQIAMFCGRLNMHMNVQNGKWDSDPSGTKTCIDTKEGILQYCQEVYPELQITNVVEANQPVTIQNWCKRGRKQCKTHPHFVIPYRCLVGEFVSDALLVPDKCKFLHQERMDVCETHLHWHTVAKETCSEKSTNLHDYGMLLPCGIDKFRGVEFVCCPLAEESDNVDSADAEEDDSDVWWGGADTDYADGSEDKVVEVAEEEEVAEVEEEEADDDEDDEDGDEVEEEAEEPYEEATERTTSIATTTTTTTESVEEVVREVCSEQAETGPCRAMISRWYFDVTEGKCAPFFYGGCGGNRNNFDTEEYCMAVCGSAMSQSLLKTTQEPLARDPVKLPTTAASTPDAVDKYLETPGDENEHAHFQKAKERLEAKHRERMSQVMREWEEAERQAKNLPKADKKAVIQHFQEKVESLEQEAANERQQLVETHMARVEAMLNDRRRLALENYITALQAVPPRPRHVFNMLKKYVRAEQKDRQHTLKHFEHVRMVDPKKAAQIRSQVMTHLRVIYERMNQSLSLLYNVPAVAEEIQDEVDELLQKEQNYSDDVLANMISEPRISYGNDALMPSLTETKTTVELLPVNGEFSLDDLQPWHSFGADSVPANTENEVEPVDARPAADRGLTTRPGSGLTNIKTEEISEVKMDAEFRHDSGYEVHHQKLVFFAEDVGSNKGAIIGLMVGGVVIATVIVITLVMLKKKQYTSIHHGVVEVDAAVTPEERHLSKMQQNGYENPTYKFFEQMQN

SEQ ID NO: 89 - protein, 49 aa, synthetic construct
  REGION 1..49: note = Description of sequence: Amyloid-beta
DAEFRHDSGYEVHHQKLVFFAEDVGSNKGAIIGLMVGGVVIATVIVITL